(19) United States
(12) Patent Application Publication
(10) Pub. No.: US 2024/0099880 A1
(43) Pub. Date: Mar. 28, 2024

(54) THERMAL DEVICES

(71) Applicant: Relief Technologies, Inc., San Francisco, CA (US)

(72) Inventors: Jonathan Moulton Thomas, San Francisco, CA (US); Brian James Krieger, San Francisco, CA (US); Richard Thomas Caligaris, Los Altos, CA (US); Elizabeth Ann Miracle, San Francisco, CA (US); Grace Hina Lee, San Francisco, CA (US)

(73) Assignee: Relief Technologies, Inc., San Francisco, CA (US)

(21) Appl. No.: 18/530,714

(22) Filed: Dec. 6, 2023

Related U.S. Application Data
(63) Continuation of application No. 16/027,713, filed on Jul. 5, 2018.

(51) Int. Cl. A61F 7/08; A61F 7/00; A61F 7/10

(52) U.S. Cl. CPC A61F 7/08; A61F 7/007; A61F 7/10; A61F 2007/0004; A61F 2007/0024; A61F 2007/0027; A61F 2007/0035; A61F 2007/0039; A61F 2007/0075; A61F 2007/0078; A61F 2007/0087; A61F 2007/0094; A61F 2007/0296

(57) ABSTRACT

A thermal device includes a first thermal unit, a second thermal unit, and device electronics. The first thermal unit includes a first plurality of semiconductor elements sandwiched between first and second thermal unit substrates. The first thermal unit substrate exchanges heat with a user. The second thermal unit includes a second plurality of semiconductor elements sandwiched between third and fourth thermal unit substrates. The third thermal unit substrate exchanges heat with the user. The device electronics are coupled to the first thermal unit and the second thermal unit. The device electronics operate the first thermal unit in a heating state in which the first thermal unit transfers heat to the user via the first thermal unit substrate. The device electronics operate the second thermal unit in a cooling state in which the second thermal unit removes heat from the user via the third thermal unit substrate.

### CROSS-REFERENCE TO RELATED APPLICATIONS

This application is a continuation of U.S. application Ser. No. 16/027,713, filed on Jul. 5, 2018, which claims the benefit of U.S. Provisional Application No. 62/529,029, filed on Jul. 6, 2017. The disclosures of the above applications are incorporated herein by reference in their entirety.

### FIELD

The present disclosure relates to thermal devices that provide heating and cooling for a user's body.

### BACKGROUND

Heating and cooling therapy can be used to provide relief/rehabilitation for a variety of ailments, such as muscle ailments (e.g., soreness, tightness, or spasms), joint ailments (e.g., stiffness or arthritis), or other tissue ailments (e.g., tissue injuries). Cooling therapy can be applied in a variety of manners, such as via direct contact with the skin (e.g., via an ice pack or ice bath). Cooling therapy may absorb heat from the affected area, which may cause vasoconstriction, decreased local metabolism and enzymatic activity, and decreased oxygen demand. The therapeutic effects of cooling may include pain relief and a reduction in swelling of the affected areas.

Heating therapy can be applied in a variety of manners, such as via direct contact with the skin (e.g., a hot cloth, pad, or hot water bath) or via infrared radiation. Heat therapy may increase tissue temperature, which may produce vasodilation that causes increased blood flow to affected areas, thereby increasing the supply of oxygen and nutrients to the affected areas. The therapeutic effects of heat may include a reduction in pain, stiffness, and inflammation in the affected areas.

### SUMMARY

In one example, the present disclosure is directed to a thermal device comprising a package substrate, device electronics, and a plurality of thermal units connected to the package substrate. Each thermal unit comprises a plurality of semiconductor elements sandwiched between a first thermal unit substrate and a second thermal unit substrate. Each thermal unit is configured to heat a user's body in response to receiving current in a first direction. Each thermal unit is configured to cool a user's body in response to receiving current in a second direction that is opposite to the first direction. The device electronics are coupled to the thermal units. The device electronics are configured to store a first thermal device profile that includes data indicating an amount of power to deliver to each of the thermal units over a period of time. The device electronics are configured to deliver power to the thermal units according to the first thermal device profile, wirelessly receive a second thermal device profile from an external computing device, and deliver power to the thermal units according to the second thermal device profile.

In another example, the present disclosure is directed to a thermal device comprising a first thermal unit, a second thermal unit, and device electronics. The first thermal unit includes a first plurality of semiconductor elements sandwiched between a first thermal unit substrate and a second thermal unit substrate. The first thermal unit substrate is configured to exchange heat with a user. The second thermal unit includes a second plurality of semiconductor elements sandwiched between a third thermal unit substrate and a fourth thermal unit substrate. The third thermal unit substrate is configured to exchange heat with the user. The device electronics are coupled to the first thermal unit and the second thermal unit. The device electronics are configured to operate the first thermal unit in a heating state in which the first thermal unit transfers heat to the user via the first thermal unit substrate. The device electronics are configured to operate the second thermal unit in a cooling state in which the second thermal unit removes heat from the user via the third thermal unit substrate.

### BRIEF DESCRIPTION OF THE DRAWINGS

The present disclosure will become more fully understood from the detailed description and the accompanying drawings.

FIGS. 1A-1C illustrate a first example thermal device.

FIGS. 2A-2E illustrate example thermal units.

FIGS. 3A-3G illustrate example thermal units, device electronics, and a battery connected to a package substrate.

FIG. 4 illustrates example package substrates.

FIGS. 5A-5D illustrate fabrication of thermal units.

FIGS. 6A-6D illustrate example thermal units.

FIGS. 7A-7N illustrate example thermal reservoirs.

FIGS. 8A-8C illustrate connections between thermal units, devices electronics, and a battery.

FIG. 9 illustrates communication between a user device and a thermal device.

FIG. 10 is a functional block diagram of an example thermal device.

FIGS. 11A-11B are example current versus time graphs for a thermal device.

FIGS. 12A-12C are flow diagrams that illustrate different modes of thermal device operation.

FIG. 13 illustrates communication between a plurality of thermal devices and a remote server.

FIGS. 14A-14M illustrate arrangements of thermal reservoirs and thermal bridges along with operation of thermal units associated with the thermal reservoirs and thermal bridges.

FIGS. 15A-15L illustrate example thermal bridges that interface with one or more thermal units and the user's body.

FIGS. 16A-16J illustrate the integration of heating units and thermal units within a thermal device.

FIGS. 17A-17K illustrate example graphical user interfaces (GUIs) on a user device in communication with a thermal device.

FIGS. 18A-24B illustrate additional example thermal devices.

FIGS. 25A-25E illustrate example sleeves and garments that hold thermal devices.

In the drawings, reference numbers may be reused to identify similar and/or identical elements.

### DETAILED DESCRIPTION

A thermal device 100 of the present disclosure may be used to provide relief for a variety of different conditions including, but not limited to, muscle soreness, headaches, joint pain, burns, and arthritis. The thermal device 100 may also be used to provide relief for pelvic pain conditions and other conditions, such as chronic pelvic pain, dyspareunia, vulvodynia, endometriosis, dysmenorrhea (menstrual pain), and hemorrhoid discomfort.

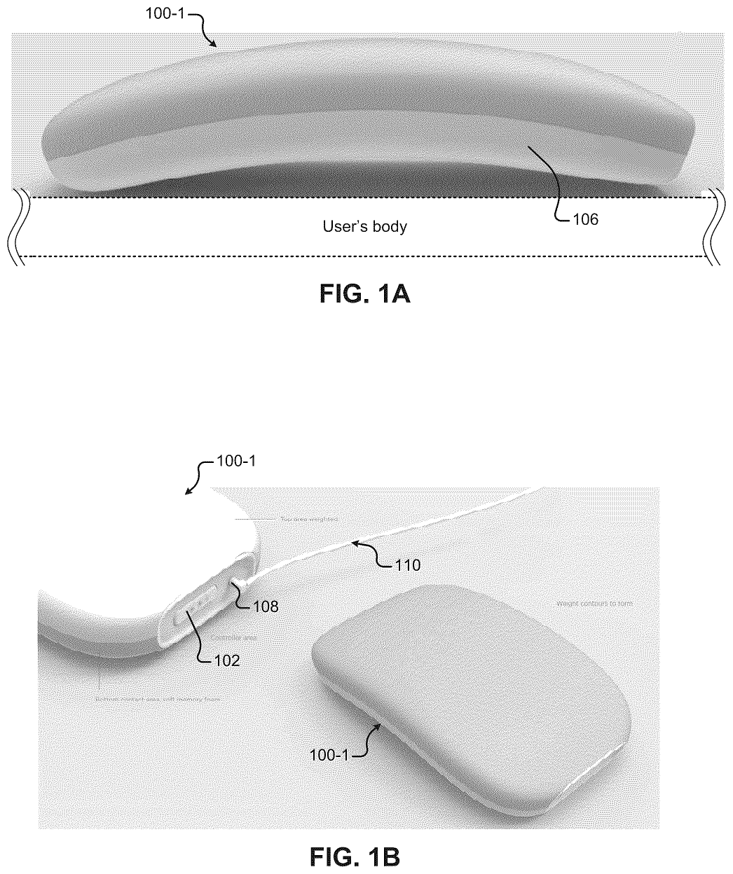

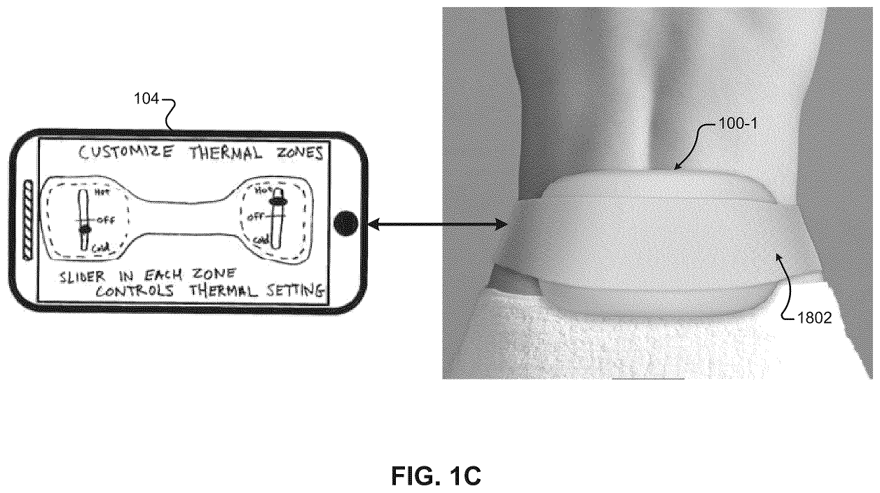

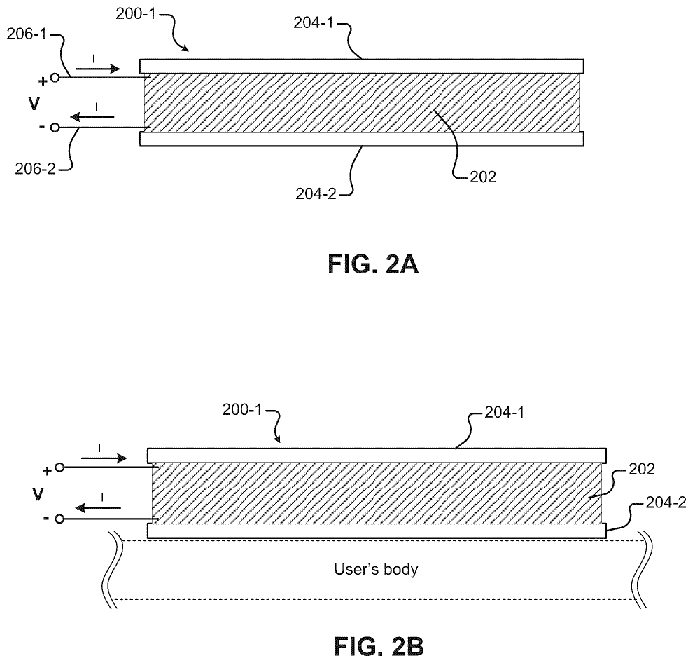

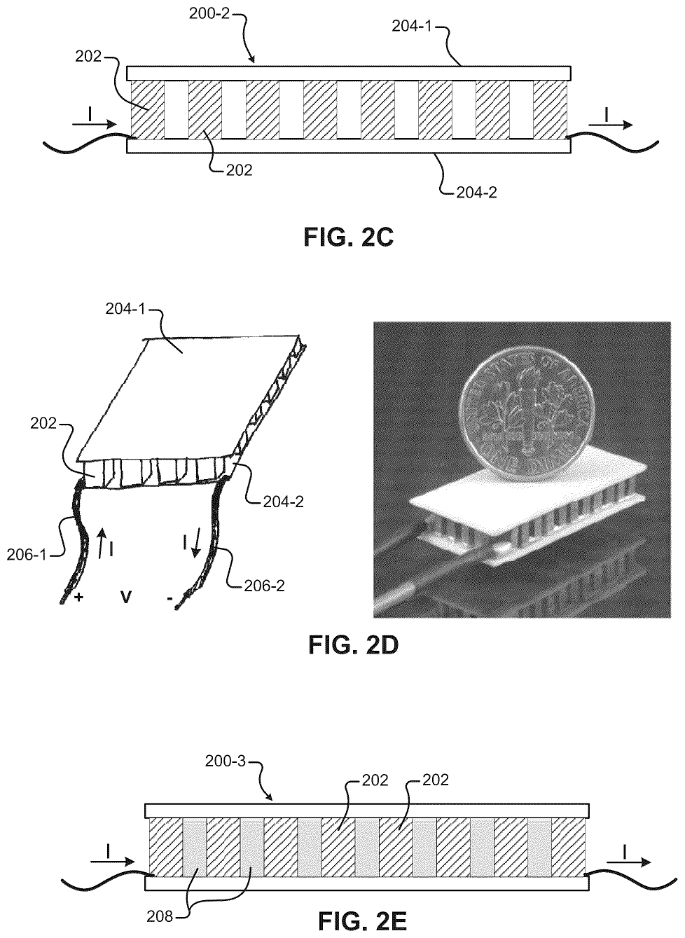

A thermal device 100 (e.g., a thermal pad) of the present disclosure includes one or more thermal units 200 that can heat and/or cool one or more areas of a user's body. The thermal device 100 can include a device package that houses the one or more thermal units 200. Example thermal devices 100-1, 100-2, . . . , 100-8 are illustrated in FIGS. 1A-1C and FIGS. 18A-24B. Example thermal units 200-1, 200-2, . . . , 200-9 are illustrated in FIGS. 2A-3E and FIGS. 5D-6D. In some cases, thermal devices and thermal units may generally be indicated by callout 100 and callout 200, respectively.

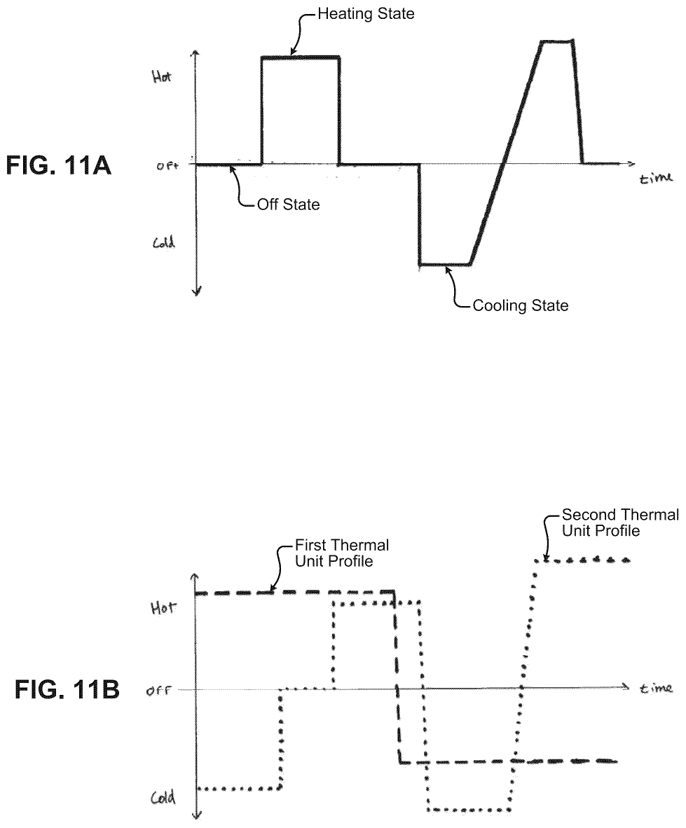

A user can control the thermal device 100 manually. For example, the thermal device 100 may include user input devices 102 (e.g., manual controls) and/or be controlled via an external computing device 104, such as a user's phone (e.g., see FIG. 1C). The thermal device 100 may also automatically execute thermal device profiles that include data indicating how the thermal device 100 should operate over time. (e.g., see FIGS. 11A-11B).

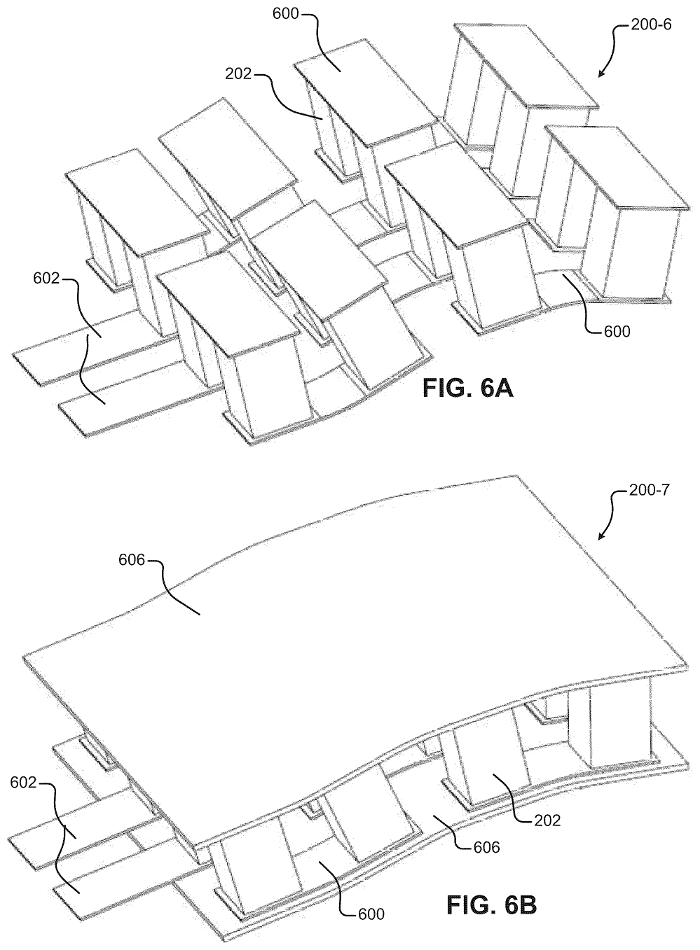

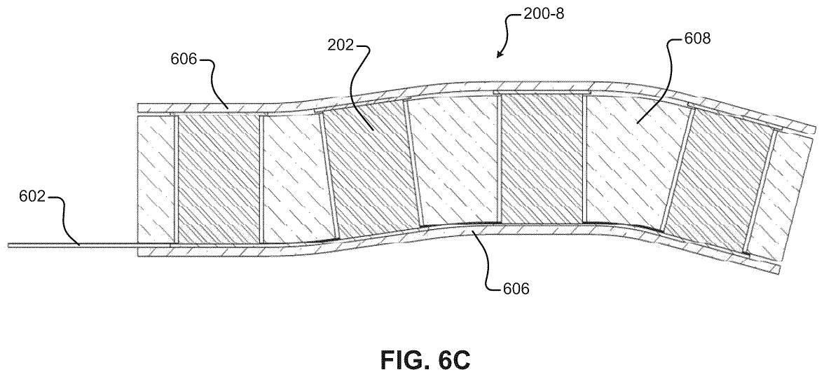

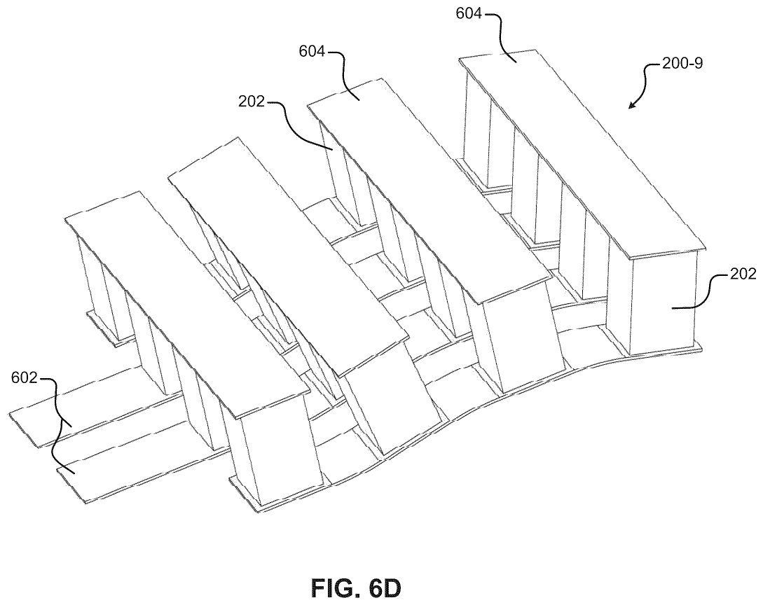

In some implementations, a thermal unit 200 can include a plurality of thermal elements 202 (e.g., semiconductor elements) sandwiched between two thermal unit substrates 204, which may be flexible and/or rigid. A thermal unit 200 can also include electrical contacts (e.g., 206-1, 206-2 of FIG. 2A) for connecting to device electronics included in the thermal device 100. In some implementations, the thermal elements 202 may be connected in series between the electrical contacts such that current can be delivered through the thermal elements 202 via the electrical contacts (e.g., see FIG. 6A). In some implementations, the thermal unit 200 may be a thermoelectric device (e.g., a solid-state heat pump) that includes semiconductor thermal elements mounted between two substrates (e.g., ceramic substrates). Individual thermal units may be arranged on a device package substrate (e.g., package substrate 300) included in the thermal device 100. The device package can provide support to the thermal units 200 so that the thermal units 200 can be positioned near the user's body. The arrangement of the thermal units 200 may define different thermal zones of the thermal device 100.

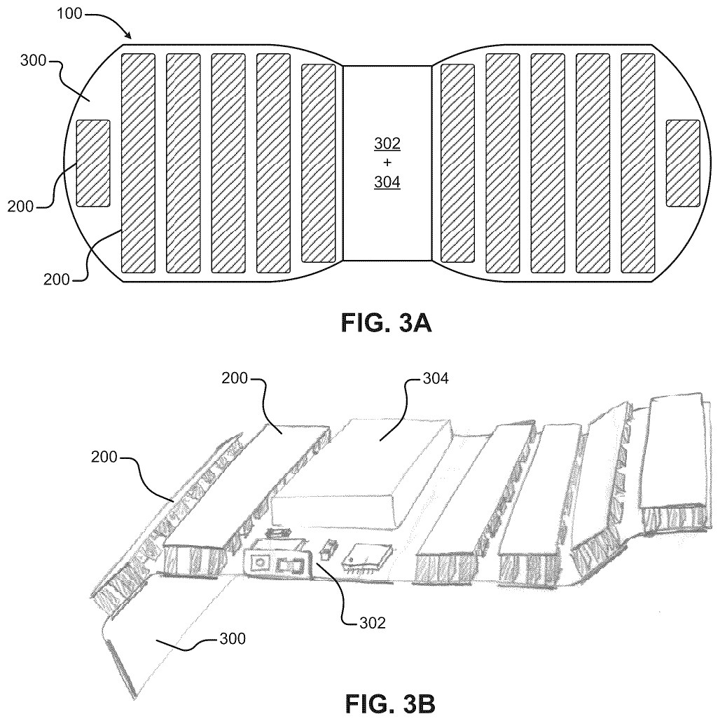

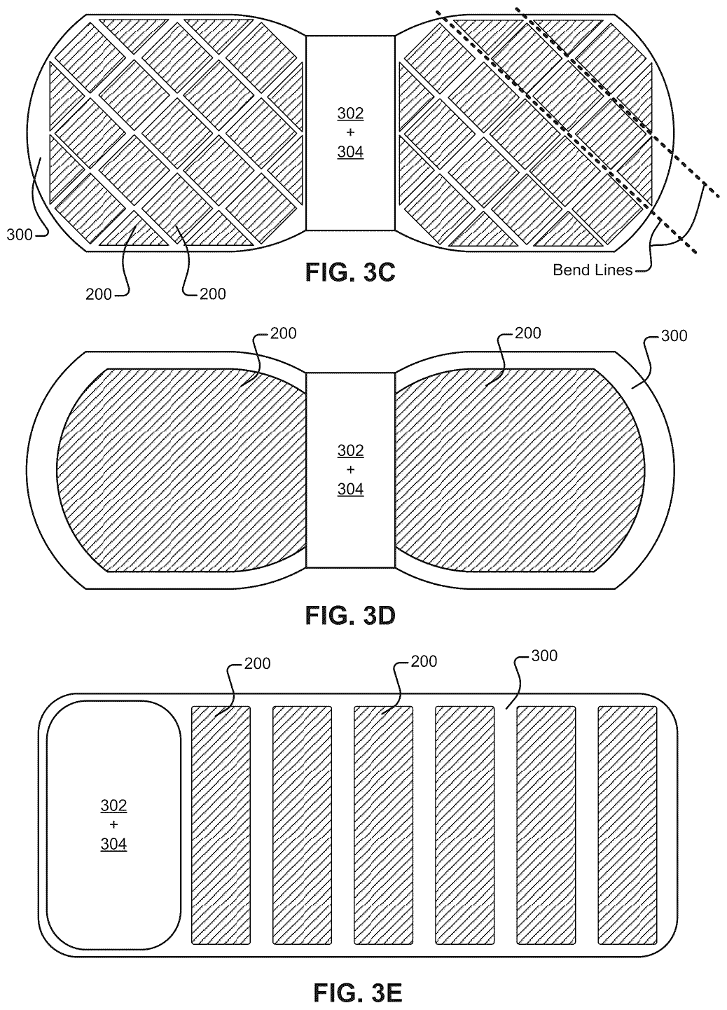

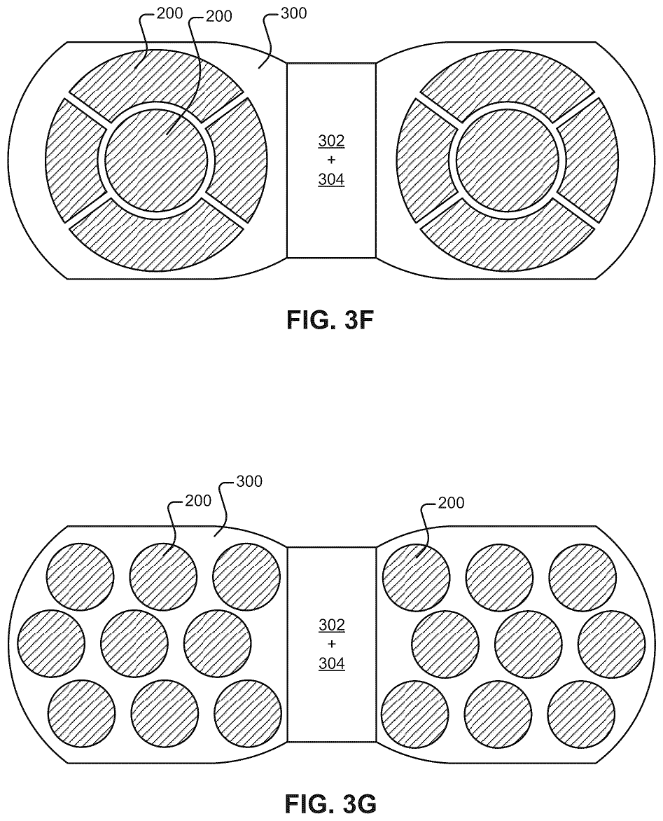

The thermal device 100 includes device electronics (e.g., at 302 in FIGS. 3A-3B) that control the thermal units 200. For example, the device electronics may control the thermal units 200 by controlling power (e.g., current/voltage) delivered to the thermal units 200 via the electrical contacts on the thermal units 200. The device electronics may increase/decrease the amount of power applied to a thermal unit 200 to increase/decrease the amount of heating or cooling provided by the thermal unit 200. The polarity of the voltage and direction of current applied to a thermal unit 200 may control which side of the thermal unit provides heating/cooling. The side (e.g., thermal unit substrate) that provides cooling may be referred to herein as the “cold side” of the thermal unit. The side (e.g., thermal unit substrate) of the thermal unit to which heat is transferred may be referred to as the “hot side” of the thermal unit.

The device electronics may control a thermal unit 200 to operate in one of three states. The thermal unit 200 may be in the off state when the device electronics are not providing power to the thermal unit 200. A thermal unit 200 may operate in the cooling state when the device electronics are controlling the thermal unit 200 to cool the side of the thermal unit 200 in contact with the user. A thermal unit 200 may operate in a heating state when the device electronics are controlling the thermal unit 200 to heat the side of the thermal unit in contact with the user. The device electronics may transition a thermal unit 200 between the different states (e.g., according to a thermal profile). As such, the device electronics can independently control the different thermal zones to heat the user, cool the user, or operate in the off state.

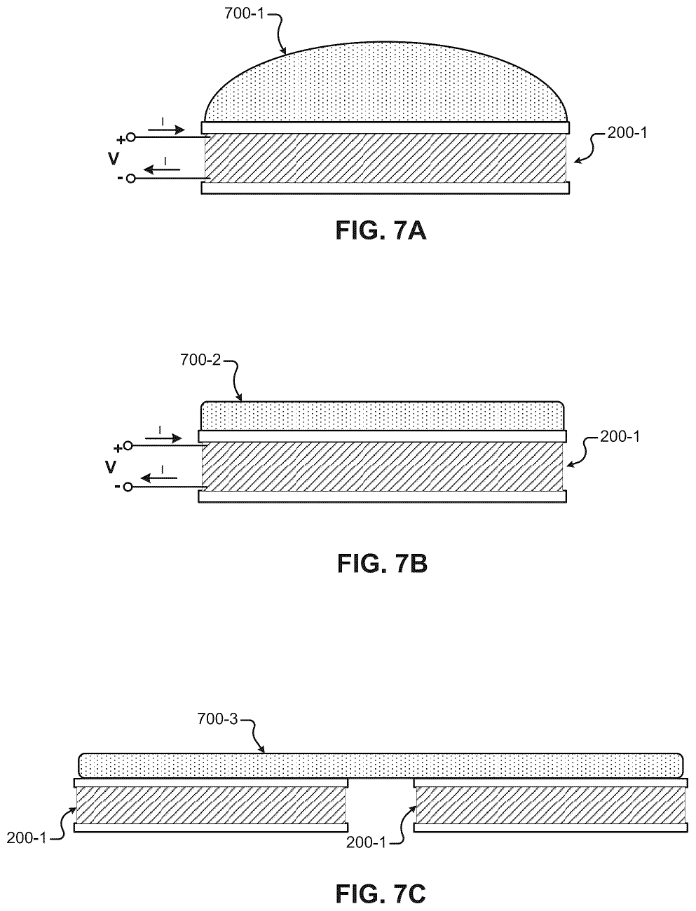

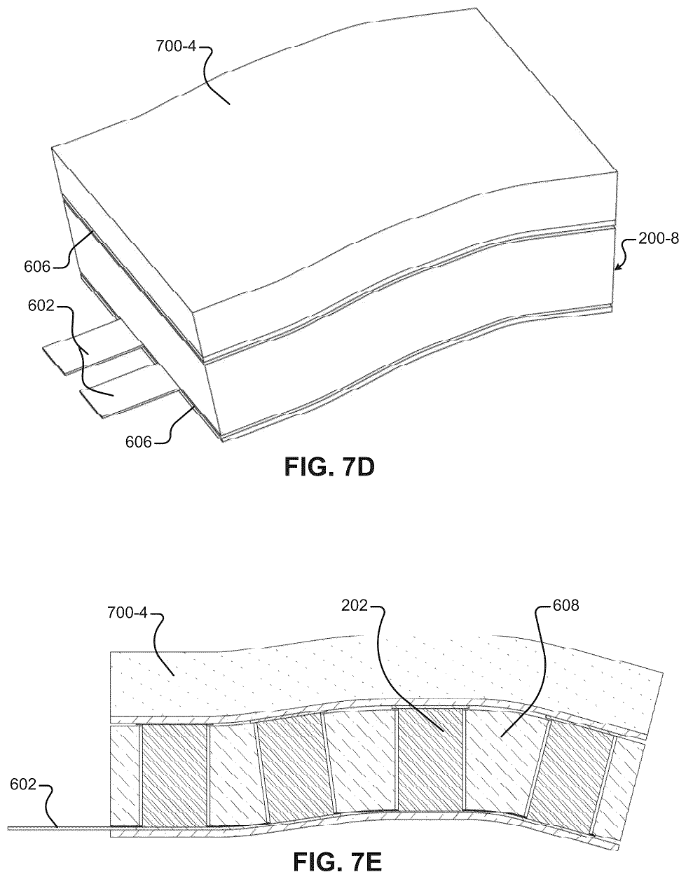

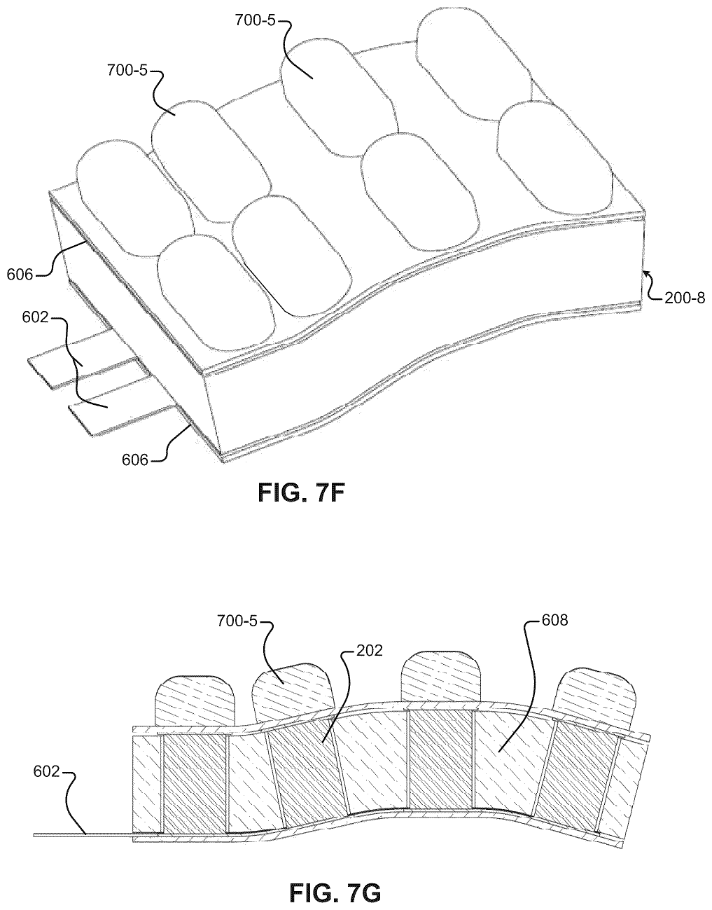

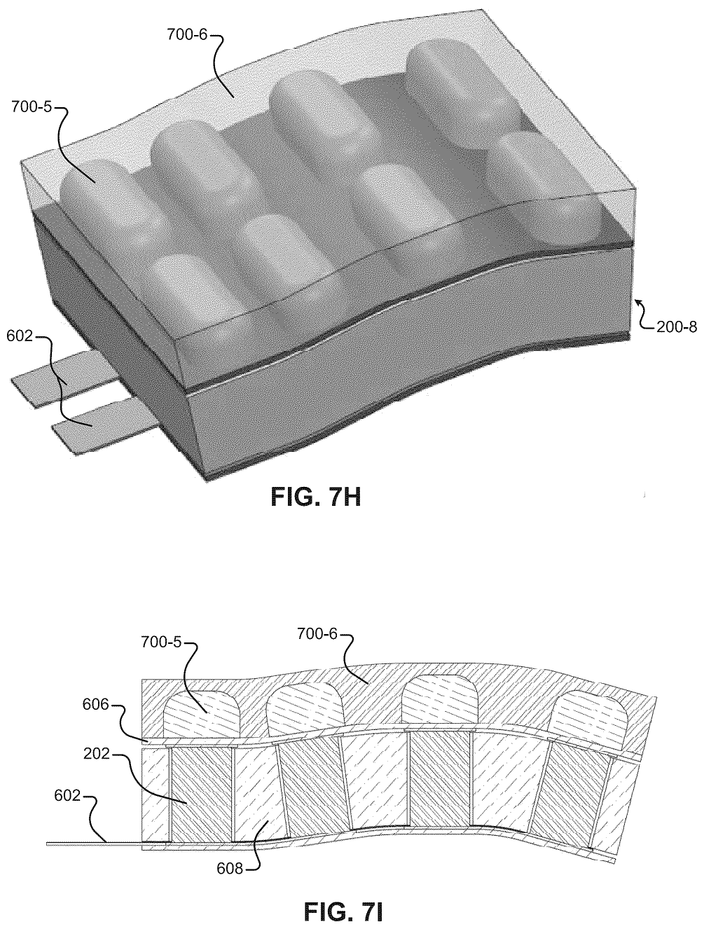

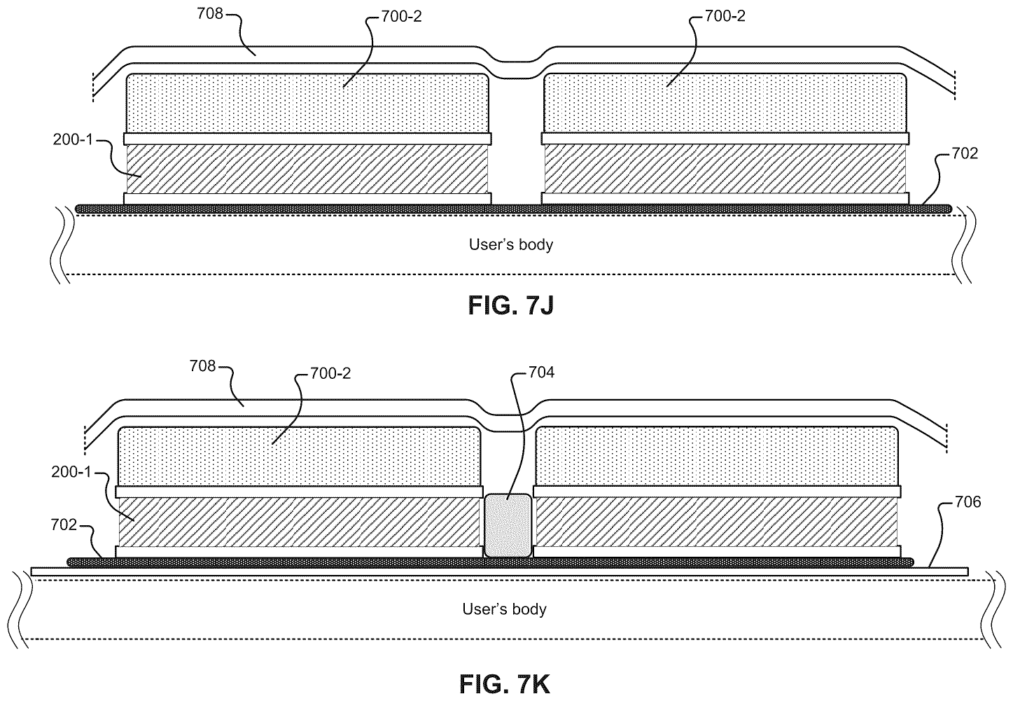

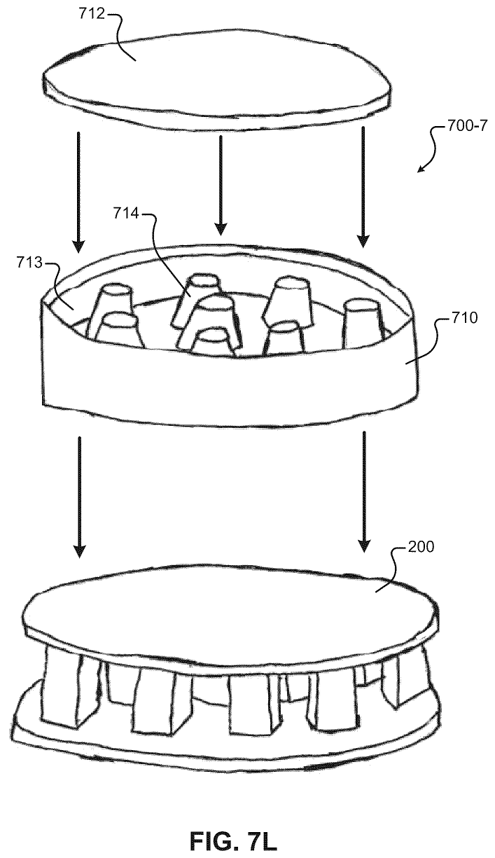

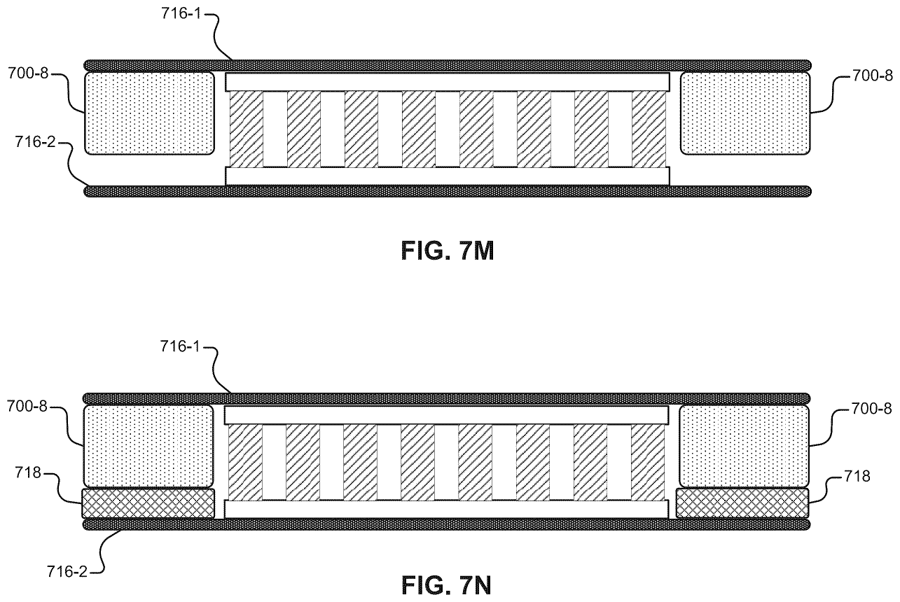

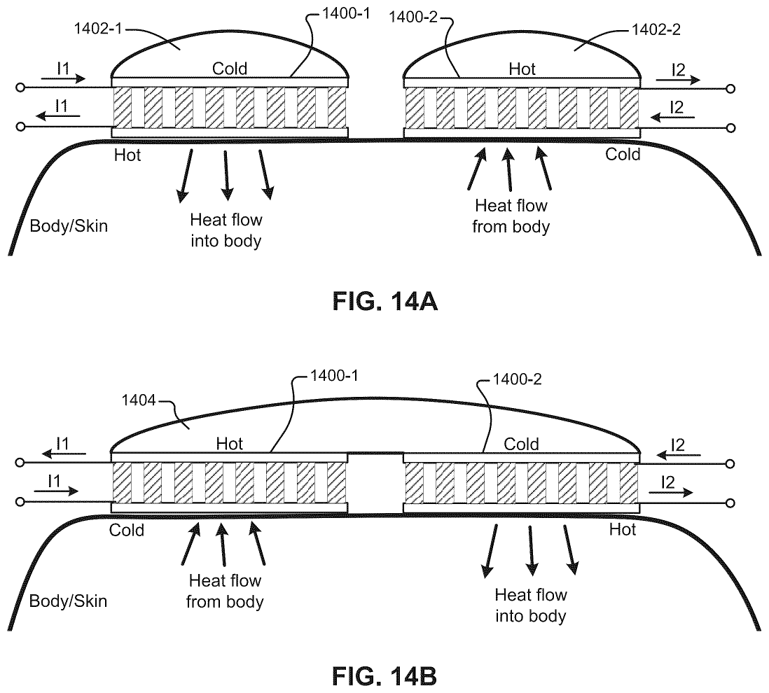

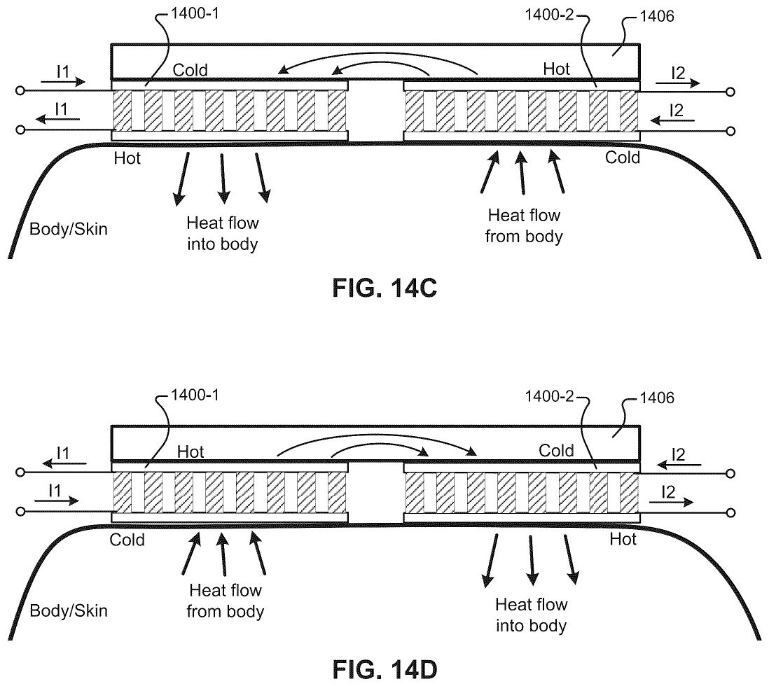

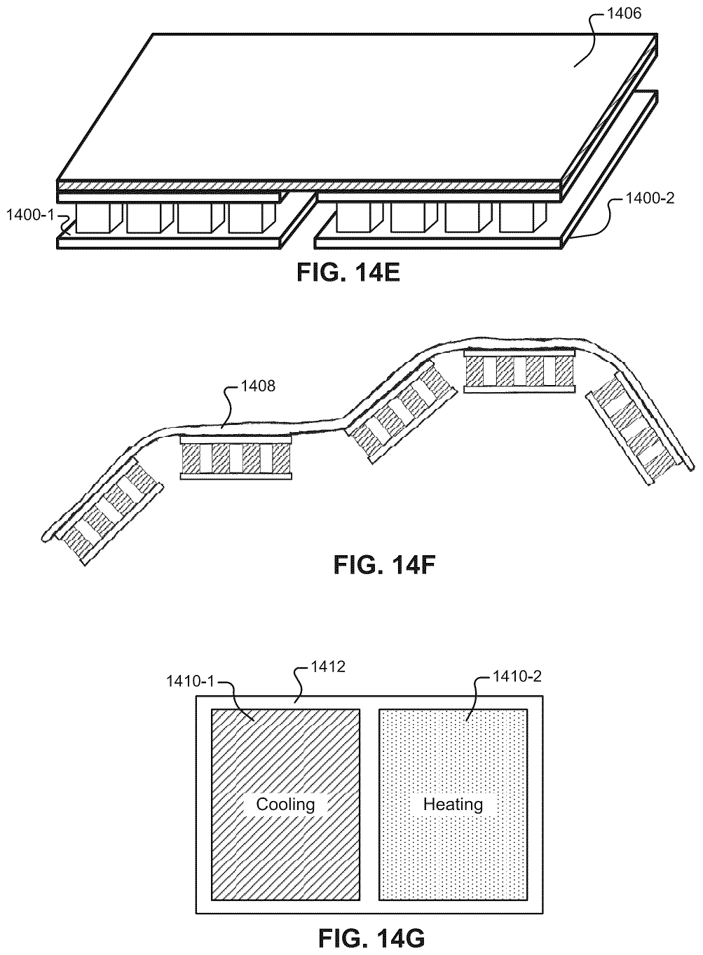

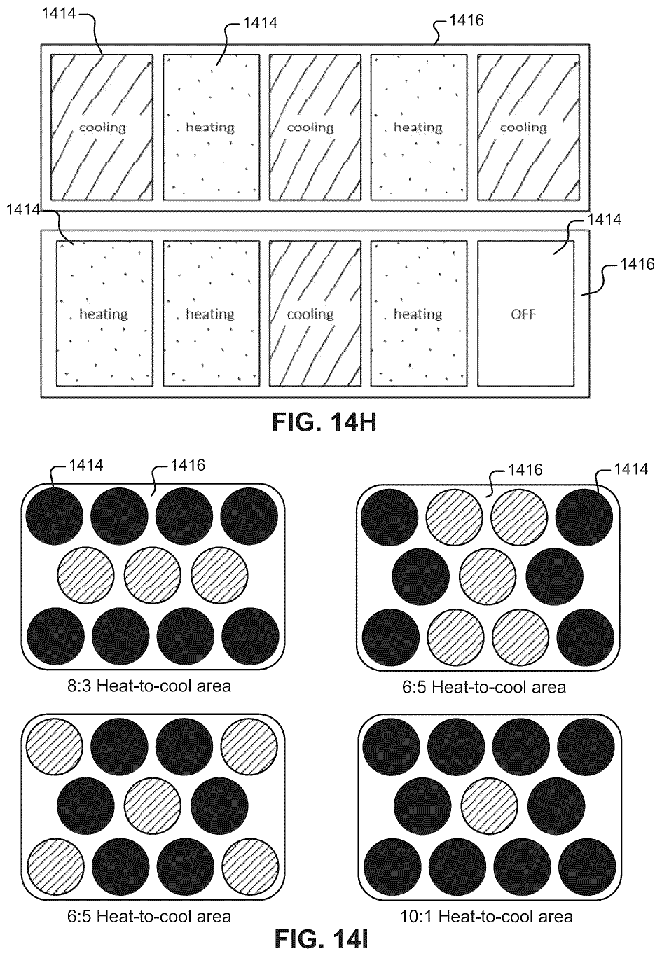

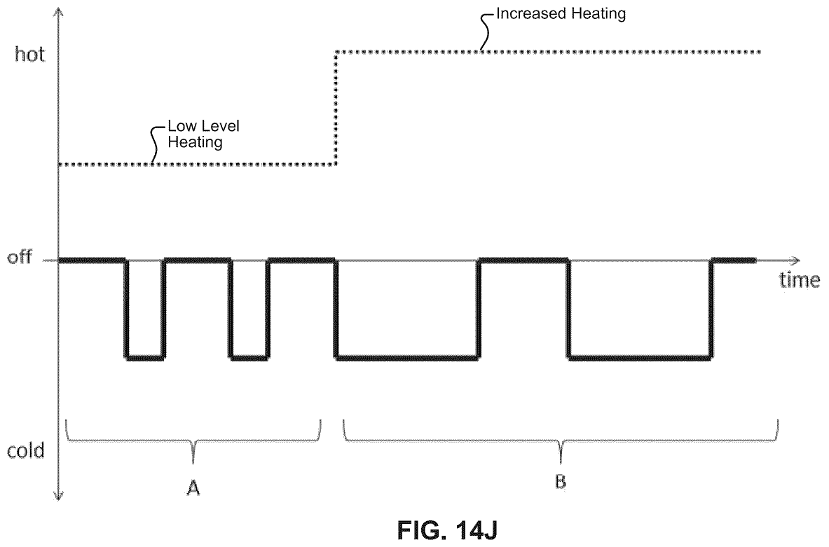

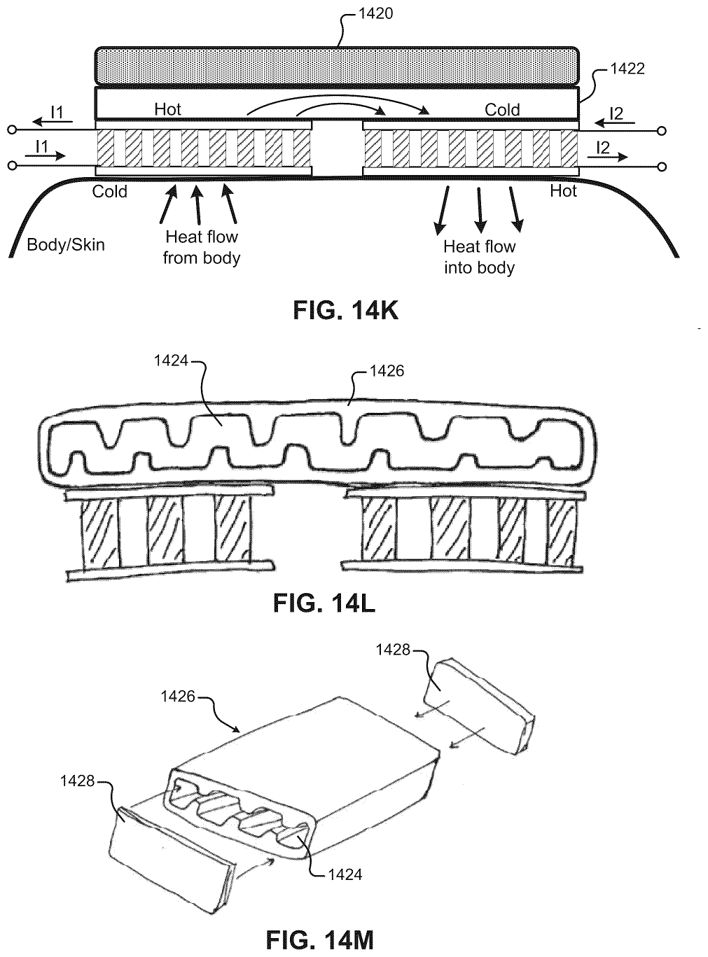

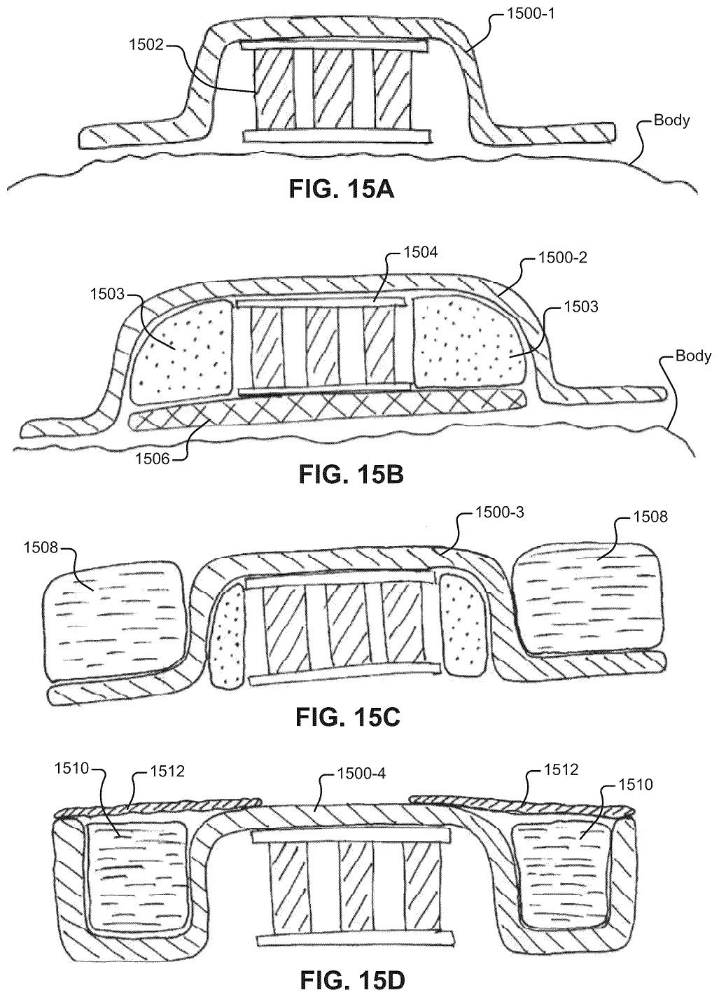

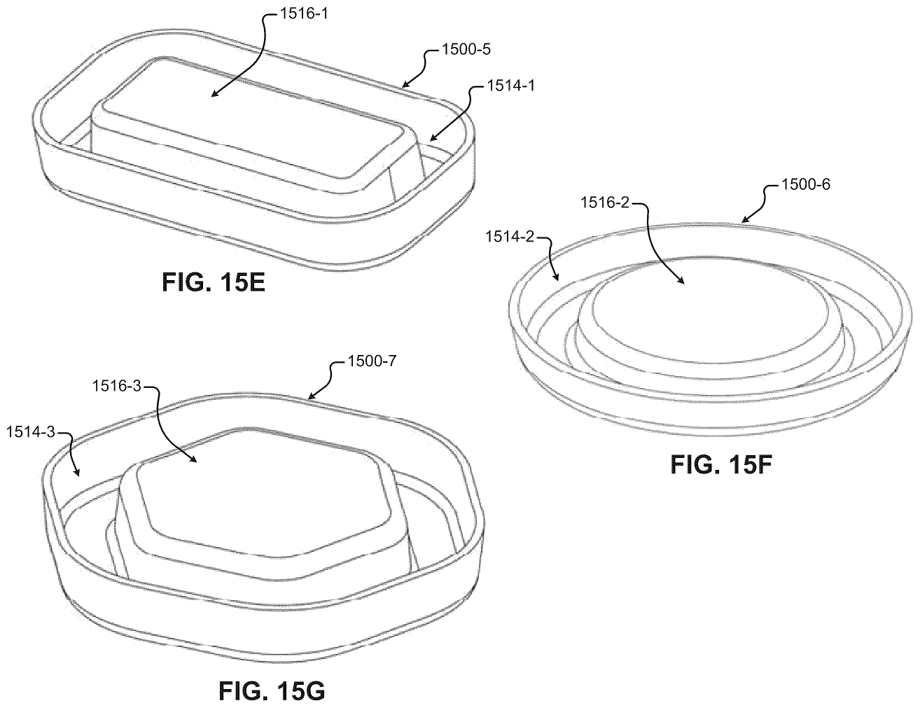

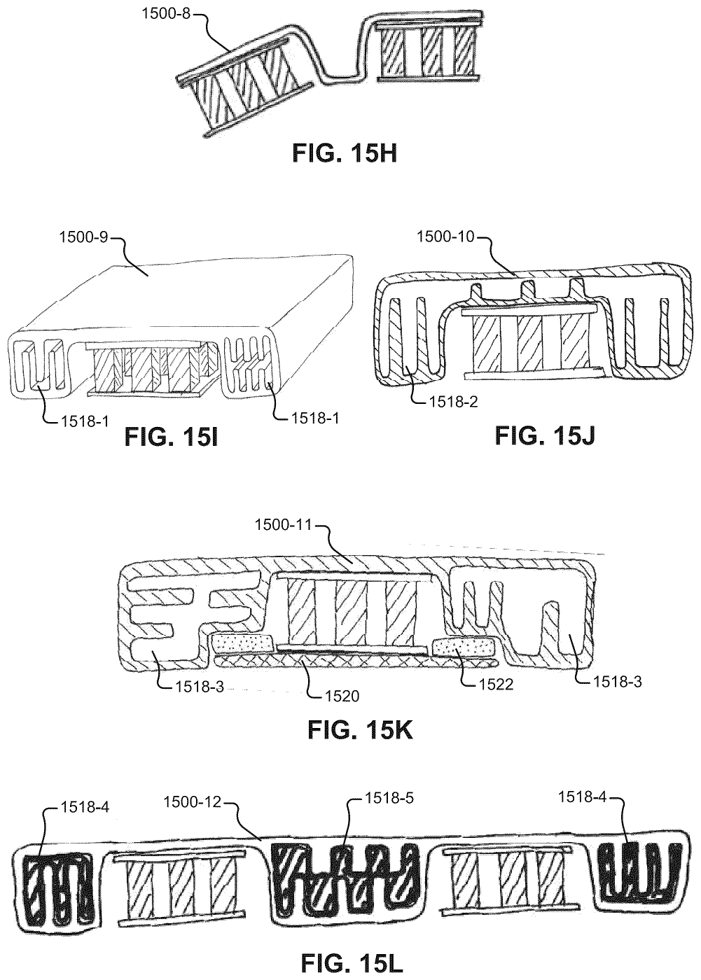

In some implementations, the thermal device 100 may include one or more thermal reservoirs (e.g., see FIGS. 7A-7N). A thermal reservoir (e.g., a phase-change material) may act as an energy storage material that may transfer heat (e.g., sink or source heat) with the thermal units 200 during operation of the thermal device 100. The energy storage and transfer provided by the thermal reservoir materials may allow the thermal units 200 to maintain their operating points for longer periods of time. In some implementations, the thermal device 100 may include one or more thermally conductive thermal bridges that transfer heat between thermal units 200 and/or the user's body during operation of the thermal device 100 (e.g., see FIGS. 14C-15L). For example, the thermal bridges may be used to create a “thermal circuit” in which heat withdrawn from the body by a first thermal unit is transferred to a second thermal unit that is transferring heat into the body. In some implementations, the thermal reservoirs may be added to the thermal bridges in order to sink/source heat from the thermal bridge.

In some implementations, the thermal device 100 may include one or more sensors (e.g., temperature, orientation, motion, and/or pressure sensors). In these implementations, the device electronics may control heating/cooling based on data acquired from the one or more sensors. In some implementations, the thermal device 100 may include a battery 304 (e.g., see FIGS. 3A-3B). In these implementations, the device electronics can manage charging/discharging of the battery 304 and control heating/cooling based on a variety of conditions, such as a state of charge of the battery 304, the currently running thermal device profile, and/or a target device run time indicated by the user.

In some implementations, the thermal device 100 may include user interface devices that allow the user to interact with the thermal device 100. For example, the thermal device 100 may include buttons, switches, touch sensitive controls, and/or a display that allow the user to control/monitor the amount of heating/cooling. The device electronics may communicate with the user interface devices in order to control heating/cooling and provide output to the user. In some implementations, the device electronics may include electronics that can communicate with an external wired/wireless computing device, such as a user's cell phone (e.g., see FIG. 1C). In these implementations, the user may control and monitor heating/cooling using the external computing device. The external computing device may be referred to herein as a “user device.”

The thermal device 100 can be powered in a variety of different ways. In some implementations, the thermal device 100 can be configured to receive a battery 304 (e.g., rechargeable/non-rechargeable battery) from the user. The battery 304 may be removable by hand and/or fixed within the thermal device 100 (e.g., accessible using tools). Additionally, or alternatively, the thermal device 100 can be plugged into an external power source (e.g., via a power input port) that may power the thermal device 100 and/or charge the battery 304.

The arrangement of the one or more thermal units 200 may create one or more thermal zones. A thermal zone refers to an area of the thermal device 100 in which the thermal device heats/cools the user. A user may control heating/cooling in a thermal zone by controlling power delivered to the thermal unit(s) 200 making up the thermal zone. In some cases, thermal zones can be surrounded by areas of the thermal device 100 not including thermal units 200. Put another way, if a thermal device 100 has multiple thermal zones, the thermal zones can be separated from one another. In other cases, the thermal zones may not be separated, but instead, some of the thermal zones may merge together.

The thermal device 100 can be configured to operate in one or more of three modes, which may be referred to herein as a manual mode, an automatic mode, or a mixed mode. The thermal device 100 can operate in a manual mode in which the thermal device 100 is configured to heat/cool in response to a user's manual input. For example, while operating in the manual mode, a user can control heating/cooling using manual controls on the thermal device 100 and/or using the user device 104. In a more specific example, the user can incrementally increase/decrease heating and cooling in different thermal zones using manual controls and/or graphical controls rendered on a graphical user interface (GUI) of the user device 104. In the manual mode, the user may control one or more of the thermal zones. If the thermal device 100 has multiple thermal zones, the user may manually control the thermal zones independently or together.

The thermal device 100 can operate in an automatic mode in which the thermal device heats/cools according to a thermal profile, or sequence of profiles, loaded on the thermal device 100. The thermal profile can include data indicating how the thermal device 100 should heat/cool the one or more thermal zones. For example, if a thermal device 100 includes a single thermal unit 200, the thermal profile may include data that indicates how to control the thermal unit 200. In this example, the thermal profile may include data indicating the power (e.g., voltage/current) to be delivered to the thermal unit 200 over a period of time. FIGS. 11A-11B illustrate example thermal profiles that may be used by the thermal device 100. In thermal devices 100 including multiple thermal units 200, a thermal profile can include data indicating the power (e.g., voltage/current) to be delivered to each of the multiple thermal units 200. A thermal profile may also indicate how the thermal device 100 should operate in response to data acquired from one or more sensors included on the thermal device 100. For example, the thermal profile may indicate whether to increase/decrease the delivery of power based on a detected temperature or motion-sensitive sensor.

The thermal device 100 can store one or more thermal profiles. In some implementations, the thermal profiles may be stored permanently in memory (e.g., in a ROM), and the user can select from the thermal profiles using manual controls and/or a GUI. In some implementations, the user can load different thermal profiles onto the thermal device 100 (e.g., from the user device 104) and then select from the loaded thermal profiles.

The thermal device 100 may operate in a mixed mode during which the user can modify/update a thermal profile while the thermal device 100 is heating/cooling according to the thermal profile. Modification of the thermal profile may refer to a situation where any portion of the thermal profile is changed by the user. The user can modify the thermal profile in a variety of different ways. For example, the user may modify a thermal profile by: 1) adjusting the amount of heating/cooling (e.g., the voltage/current) by one or more thermal units 200, 2) adjusting the frequency of heating/cooling (e.g., frequency of heating/cooling pulses) in one or more thermal units 200, 3) adjusting timing delays between the one or more thermal units 200, and/or 4) loading a new thermal profile for one or more of the thermal units 200. In some mixed mode implementations, the thermal device 100 may memorize a thermal profile generated by the user. For example, the user may modify the amount of heating/cooling provided by the thermal device 100 (e.g., using the user device and/or manual controls) in one or more thermal units 200 and the thermal device 100 may store a thermal profile that corresponds to the user's heating/cooling pattern.

In some implementations, the thermal device 100 can be configured to operate in any of the three modes. For example, the thermal device 100 can be configured to allow the user to select the mode (e.g., using a button or GUI). In some implementations, the thermal device 100 can have more limited functionality. For example, the thermal device 100 may be configured to operate in one or two of the modes, but not the other mode(s). In a more specific example, the thermal device 100 may be configured to operate in the manual mode, but not the automatic or mixed modes.

The user can generate new thermal profiles in a variety of different ways. In some implementations, the user can create a new thermal profile using a computing device other than the thermal device 100, such as a cell phone or laptop computer. The user can then load the newly created thermal profile onto the thermal device 100 (e.g., using the user device 104). In some implementations, the user can create a new thermal profile from scratch (e.g., without using another existing thermal profile). In other implementations, the user can create a new thermal profile by modifying an existing thermal profile. For example, the user can modify an existing thermal profile running on the thermal device 100 (e.g., in the mixed mode) and then save the modified thermal profile as a new thermal profile. As another example, the user may load an existing thermal profile on an external computing device, modify the loaded thermal profile, and then save the modified thermal profile on the thermal device 100 as a new thermal profile. The user may also use the thermal device 100 (e.g., a user input device such as a touchscreen) to generate new thermal profiles and/or modify existing thermal profiles.

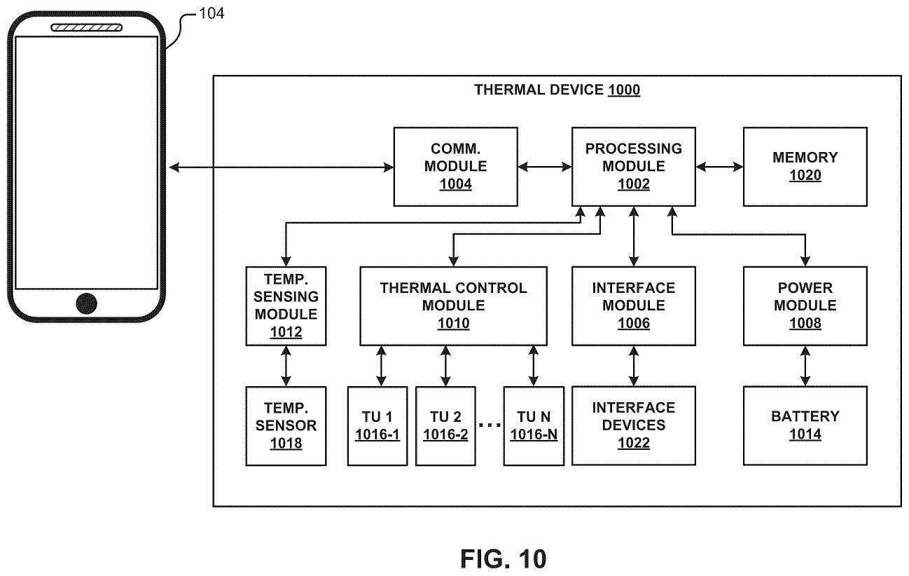

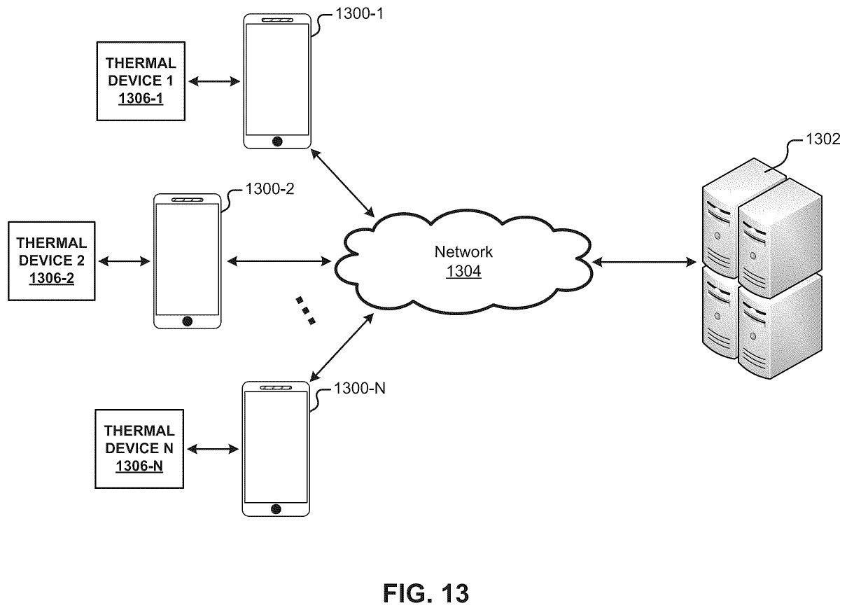

The thermal device 100 can store one or more thermal profiles in memory (e.g., memory 1020 of FIG. 10). The thermal device 100 can update the stored thermal profiles over time. For example, the thermal device 100 can delete stored thermal profiles and add additional thermal profiles to memory. The thermal device 100 can acquire thermal profiles from different sources. For example, if the thermal device 100 includes wired/wireless communication technology (e.g., WiFi, Bluetooth, USB, etc.), the thermal device 100 can retrieve thermal profiles via the internet (e.g., from the remote server 1302 of FIG. 13) and/or the user device 104.

In some implementations, the thermal device 100 includes one or more sensors. The sensors may include, but are not limited to, a temperature sensor, a motion sensor, an orientation sensor, and a pressure/force sensor. A temperature sensor may indicate the temperature of an area of the thermal device 100 in the location of the temperature sensor. Example temperature sensors may include, but are not limited to, thermocouples, thermistors, resistance temperature detectors, and semiconductor based temperature sensors. In some implementations, the thermal units 200 may be used as temperature sensors. For example, a thermal unit 200 may generate a voltage based on the temperature difference across it. A motion sensor may generate a motion signal that indicates an amount of motion of the thermal device 100 (e.g., rotation/translation). Example motion sensors may include, but are not limited to, linear or angular accelerometers, gyroscopes, magnetometers, or integrated inertial measurement units. An orientation sensor may generate an orientation signal that indicates the orientation of the thermal device 100 (e.g., indicating a user's posture). Example orientation sensors include, but are not limited to, linear or angular accelerometers, gyroscopes, magnetometers, or integrated inertial measurement units. A pressure/force sensor may indicate an amount of pressure/force in an area of the thermal device 100.

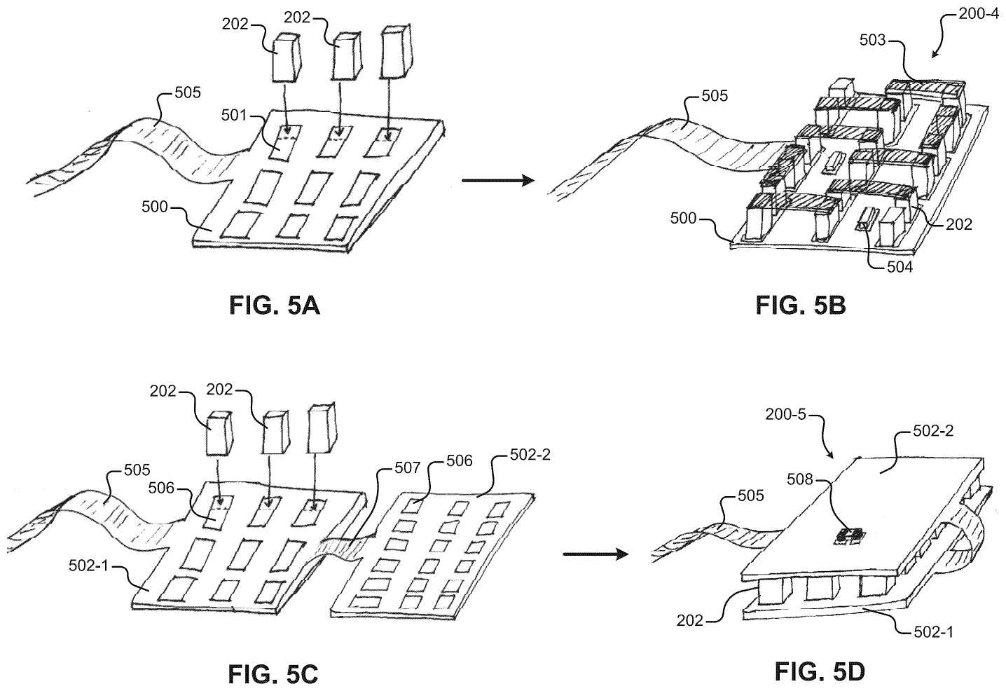

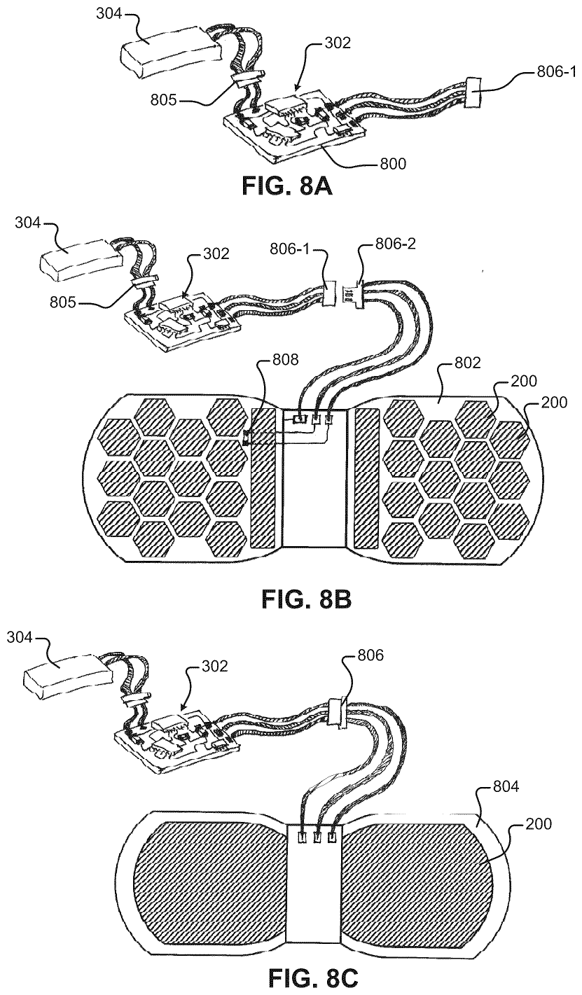

One or more sensors may be located on or within the device package. The temperature sensors may be positioned near thermal units 200 so that the temperature indicated by the temperature sensors reflect the temperature near one or more thermal units 200. Integrating the temperature sensors onto the substrates (e.g., thermal unit substrates and/or package substrates) may be beneficial in some implementations. For example, integrating a temperature sensor onto one of the substrates (e.g., FIG. 5B, FIG. 5D, and FIG. 8B) may provide for more accurate temperature sensing at the location where the user is being heating/cooled. Additionally, or alternatively, the temperature sensors may be located farther from the thermal units 200, such as along with the device electronics, which may be located off of a package substrate. In some implementations, a temperature sensor can be placed in contact with a user's body. For example, a temperature sensor may be embedded in an external portion of the device package in contact with the user's body. As another example, a temperature sensor may be attached externally to the thermal device 100 via a wire and sandwiched between the user and the thermal device during use.

The orientation/motion sensors may also be included on the substrate (e.g., thermal unit substrates and/or package substrates) and/or along with the device electronics in order to detect the orientation/motion of the thermal device 100 (i.e., the user). In some implementations, an orientation/motion sensor may be included on the user device 104 (e.g., a cell phone) which may be carried by the user (e.g., in their hand or pocket) and, therefore, detect the orientation/motion of the user. In these implementations, the user device 104 may communicate with the thermal device 100 so that the thermal device 100 can modify heating/cooling based on the user's orientation/motion as determined by the user device 104. In some implementations, orientation/motion sensors may be included in both the thermal device 100 and the user device so that the thermal device 100 can modify heating/cooling based on multiple sensors in different physical locations (e.g., based on a difference between the output of the sensors).

The device electronics may control heating/cooling based on data acquired from the sensors. For example, with respect to a temperature sensor, the device electronics may control the thermal device 100 to maintain a target temperature, maintain a temperature that is greater than a threshold temperature, or maintain a temperature that is less than a threshold temperature. With respect to the orientation/motion sensors, the device electronics may change thermal profiles or intensity based on a user's orientation and/or amount of motion. In a specific example, if a motion sensor detects changes indicative of user movement, the device electronics may be configured to increase heating/cooling to alleviate discomfort resulting from movement. In a different specific example, the device electronics may be configured to increase heating/cooling when a user is seated (e.g., as detected by the orientation/motion sensors) in order to alleviate discomfort resulting from sitting for long periods of time. In another specific example, the device electronics may be configured to reduce heating/cooling in response to reduced motion (e.g., in order to encourage user movements).

The thermal device 100 can determine a user status based on data acquired from one or more sensors. The thermal device 100 may load different thermal profiles corresponding to the different user statuses. For example, the thermal device 100 may include a seated thermal profile, a standing thermal profile, a walking thermal profile, and a running thermal profile that may be loaded in response to the thermal device 100 detecting a corresponding user status. In a specific example, if the thermal device 100 determines that a user is seated (e.g., upright posture with little motion), the thermal device 100 may load a seated thermal profile. At a later time, if the thermal device 100 detects that a user transitions from a seated position to walking, the thermal device 100 may load the walking thermal profile. The user may configure the different thermal profiles for different statuses. In some cases, the user may configure the thermal device 100 to cease heating/cooling during some user activities and provide heating/cooling during other activities. For example, the thermal device 100 may be configured to remain in a standby state (e.g., where heating/cooling is turned off) when the user is seated, and then provide heating/cooling when the user is standing. A user may configure the thermal device 100 in such a manner when the user feels little or no discomfort when seated, but then feels discomfort when standing. Additional user statuses can include user posture, such as whether the user is upright or leaning to one side. In some implementations, instead of loading a different thermal profile for a different status, the thermal device 100 can be configured to adjust parameters of the thermal profile, such as the amplitude of the heating/cooling, the frequency of heating/cooling pulses, or the phase difference between different thermal zones. In some implementations, the thermal device 100 can adjust behavior based on the physical location of the user. For example, the thermal device 100 may use different profiles depending on whether the user is at work, at home, or driving in a car.

The thermal device 100 can be configured to operate with varying degrees of autonomy with respect to a user device 104. In some implementations, the thermal device 100 can be configured to operate without any communication with the user device 104. For example, the thermal device 100 may not include wired/wireless communication technology for communicating with a user device 104. In other implementations, the thermal device 100 may be configured to communicate with the user device 104, but operate autonomously without further communication with the user device 104. For example, the thermal device 100 may be configured to receive thermal profiles from the user device 104 and then operate according to the thermal profiles without additional communication with the user device 104. In other implementations, the thermal device 100 may be configured to make intermittent communication with the user device 104 and operate according to instructions and/or thermal profiles received from the user device 104. In these examples, the thermal device 100 may intermittently communicate with the user device 104 to receive instructions, such as user-input instructions for increasing/decreasing the amount of heating/cooling. Accordingly, in some cases, the user device 104 can adjust operation of the thermal device 100 over time while the thermal device 100 is operating (e.g., in the automatic and/or mixed mode). During communication with the user device 104, the thermal device 100 may also send status updates back to the user device 104 (e.g., zone temperatures, battery status, active thermal profile, and other data).

The user device 104 and thermal device 100 can communicate using a variety of different communication protocols. In some implementations, communication between the user device 104 and the thermal device 100 may involve pairing followed by periodic polling/updating of data. The connection between the user device 104 and the thermal device 100 may be continuous (e.g., streaming data and/or control). Alternatively, the connection between the user device 104 and the thermal device 100 may be intermittent (e.g. downloading of a profile and/or instructions).

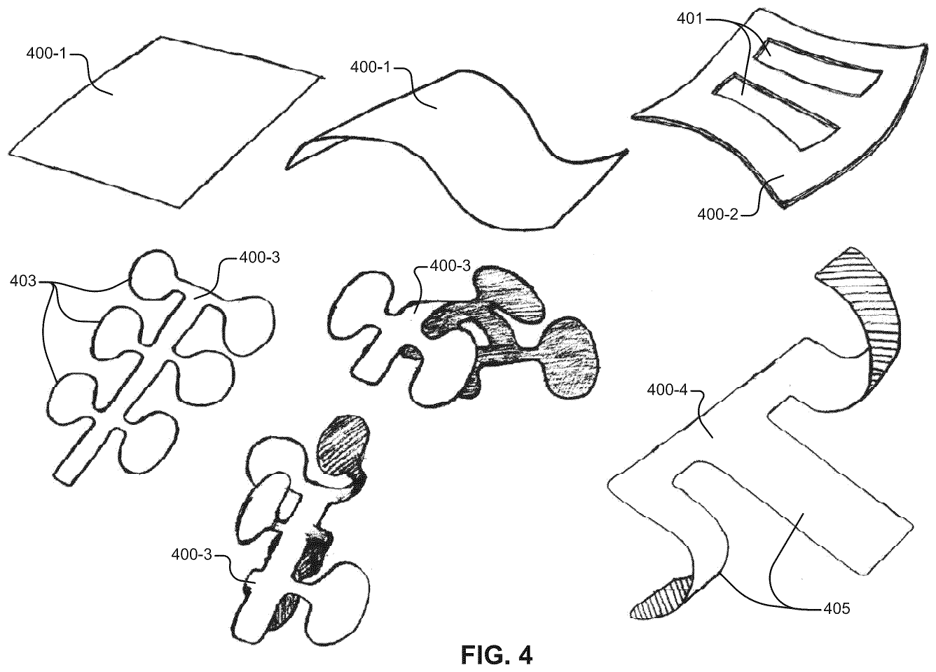

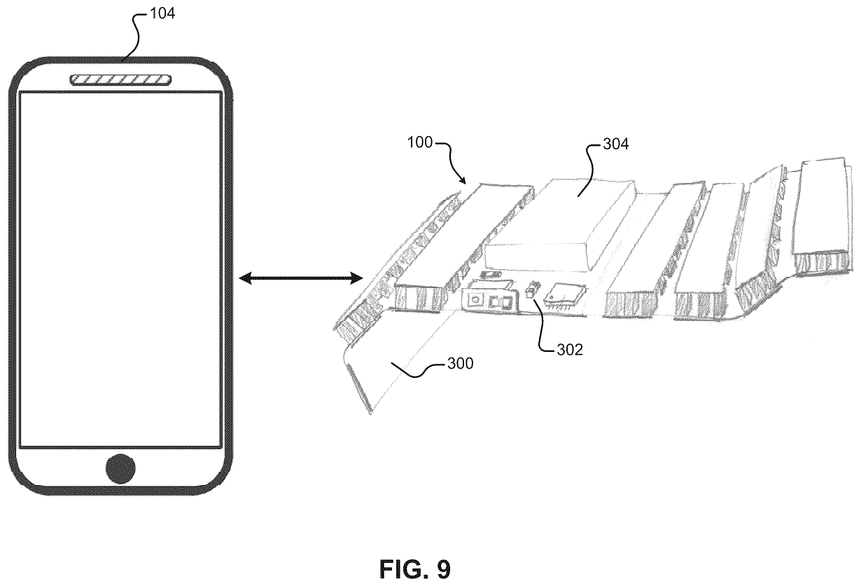

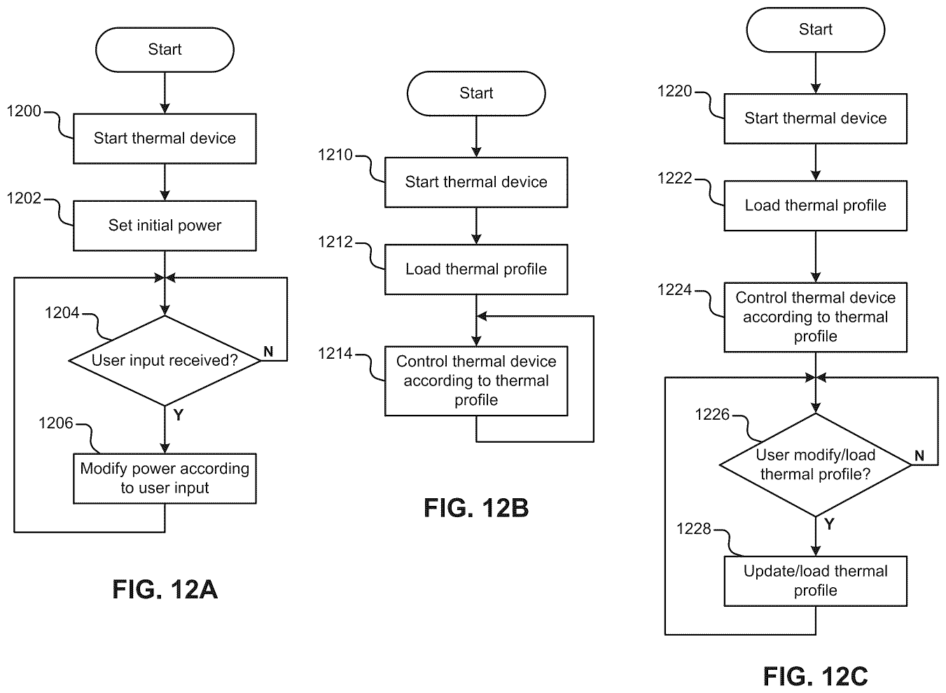

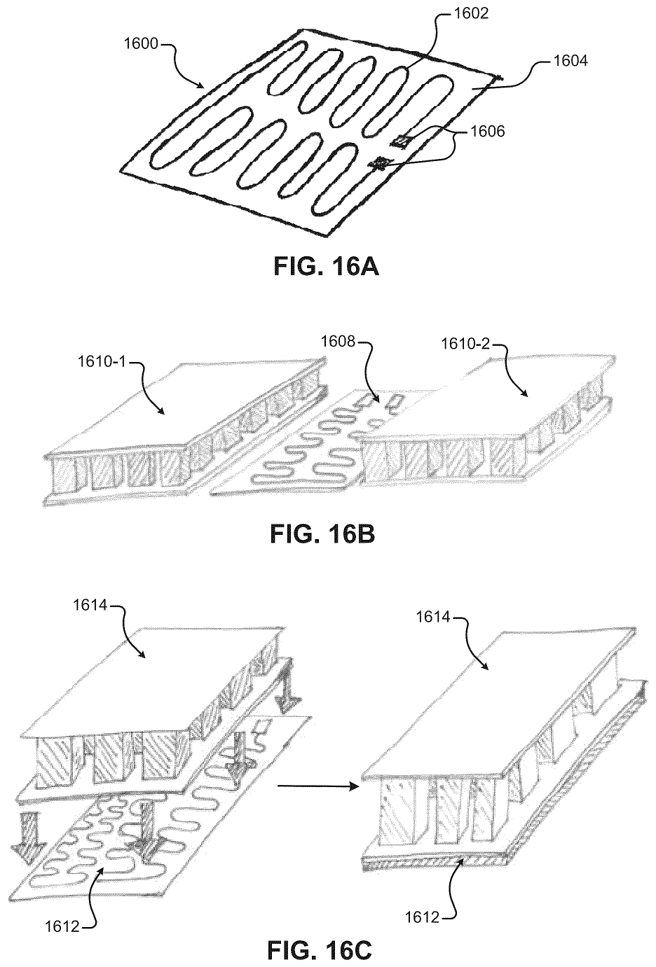

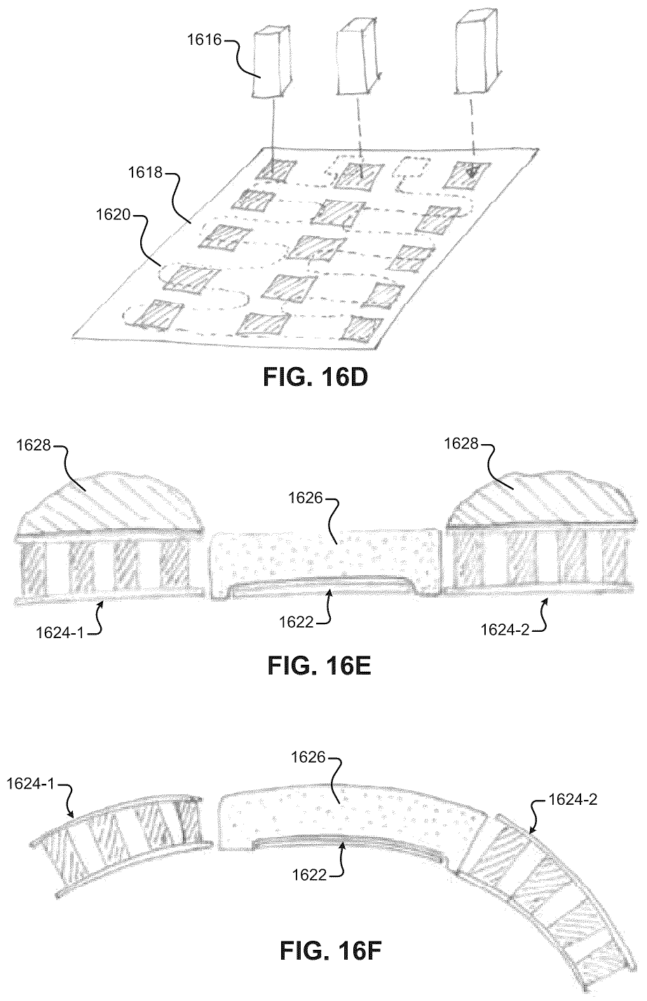

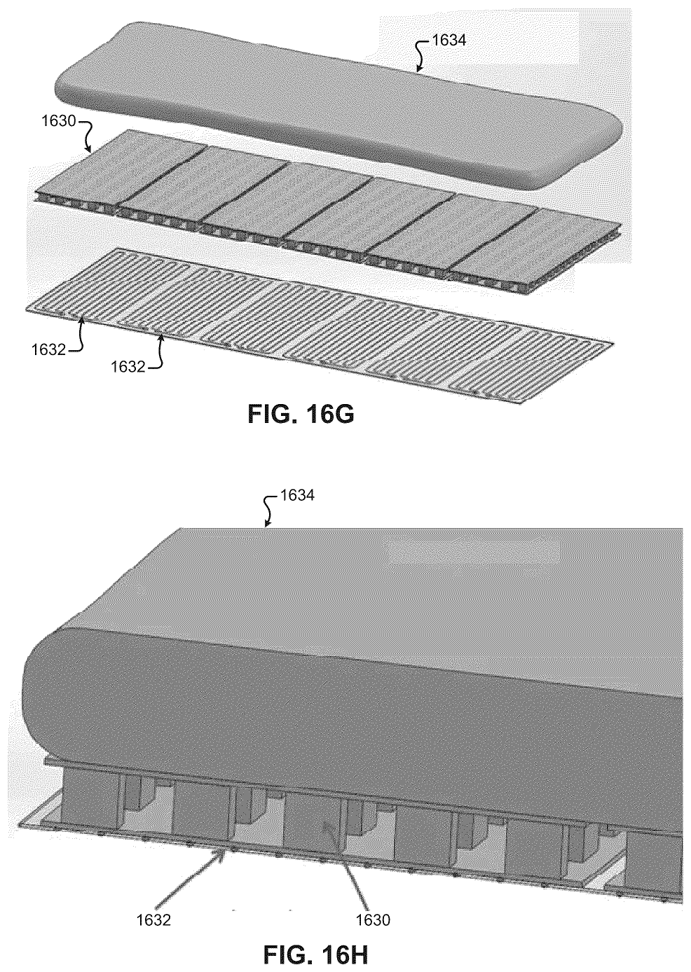

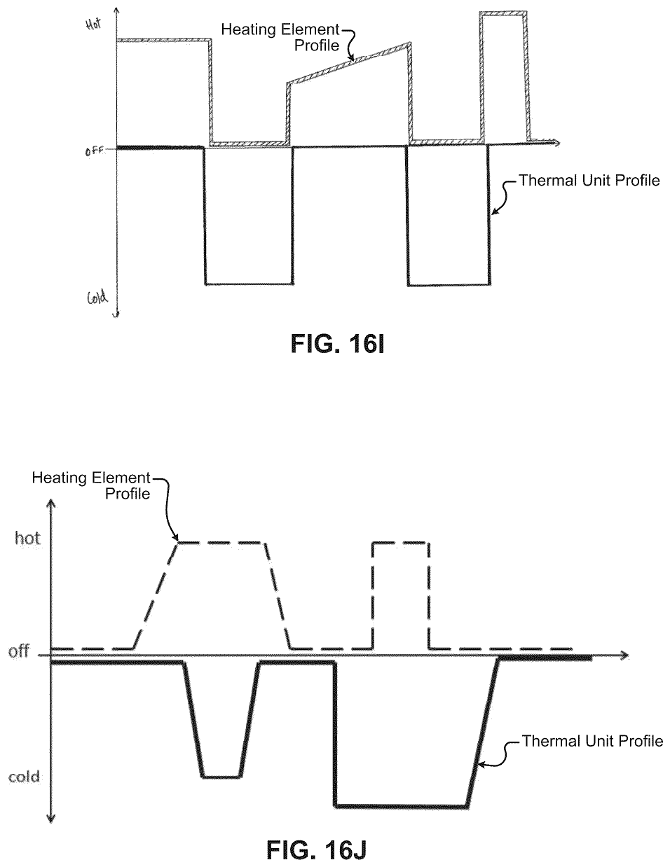

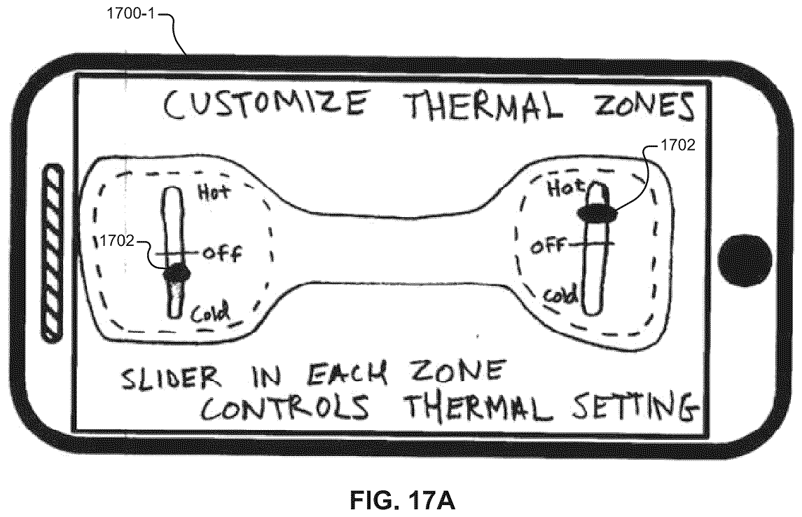

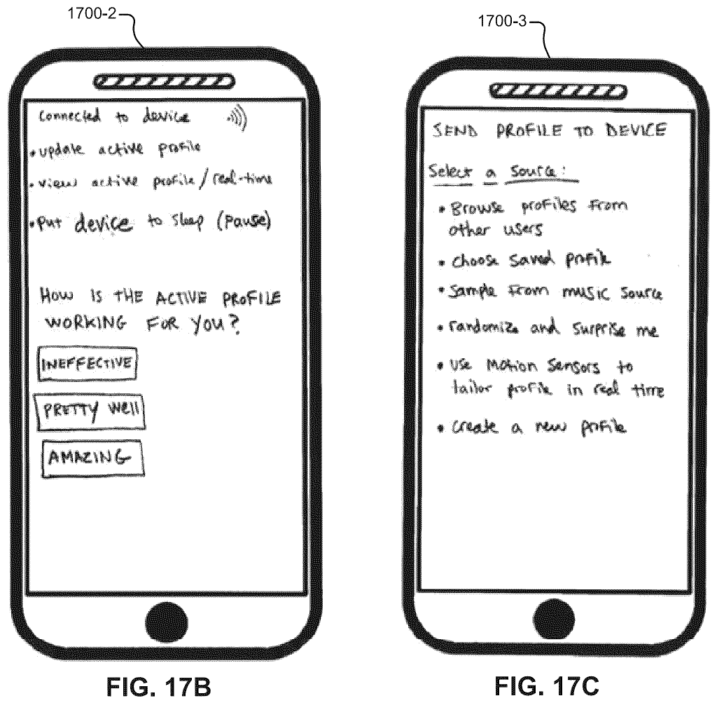

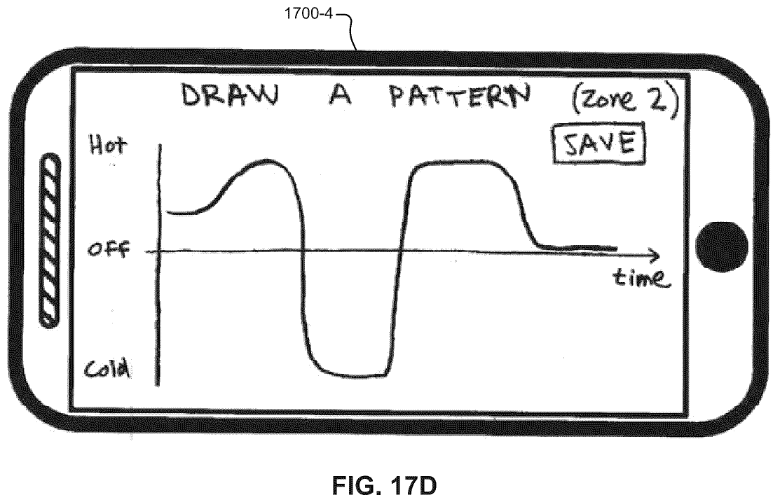

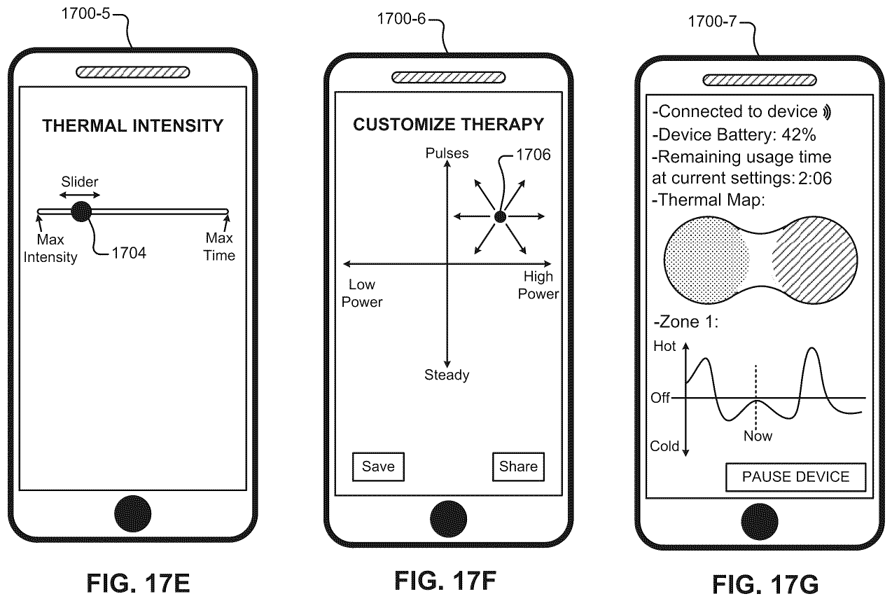

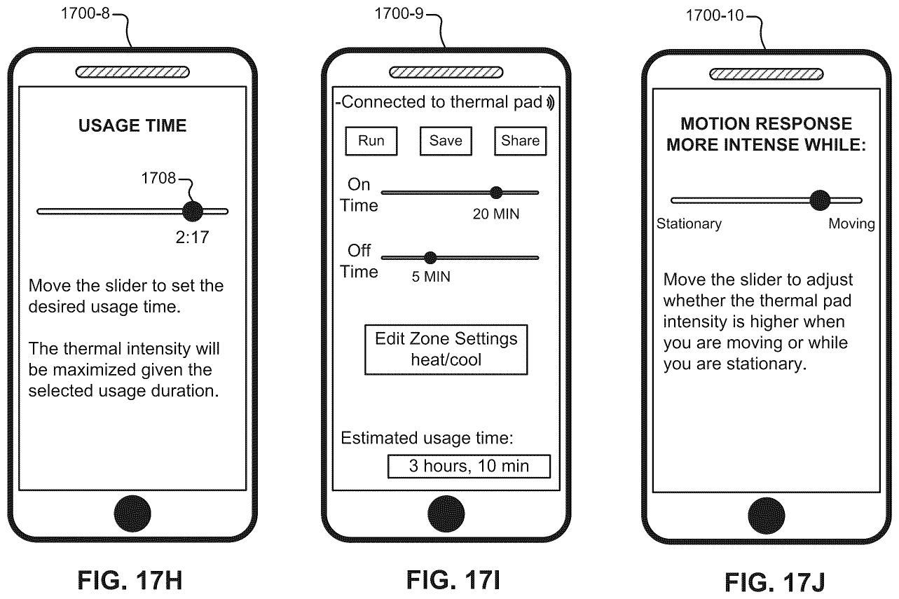

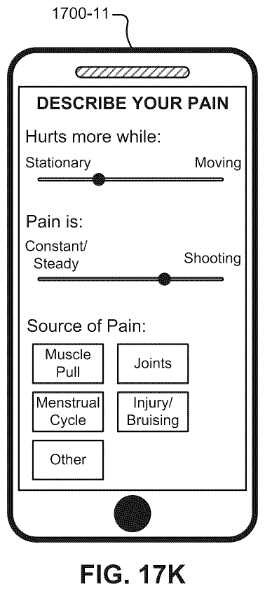

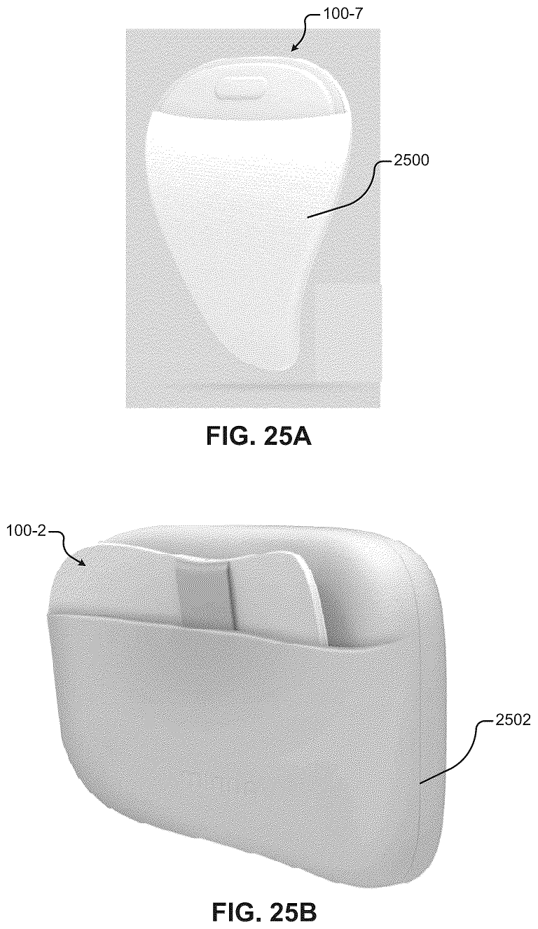

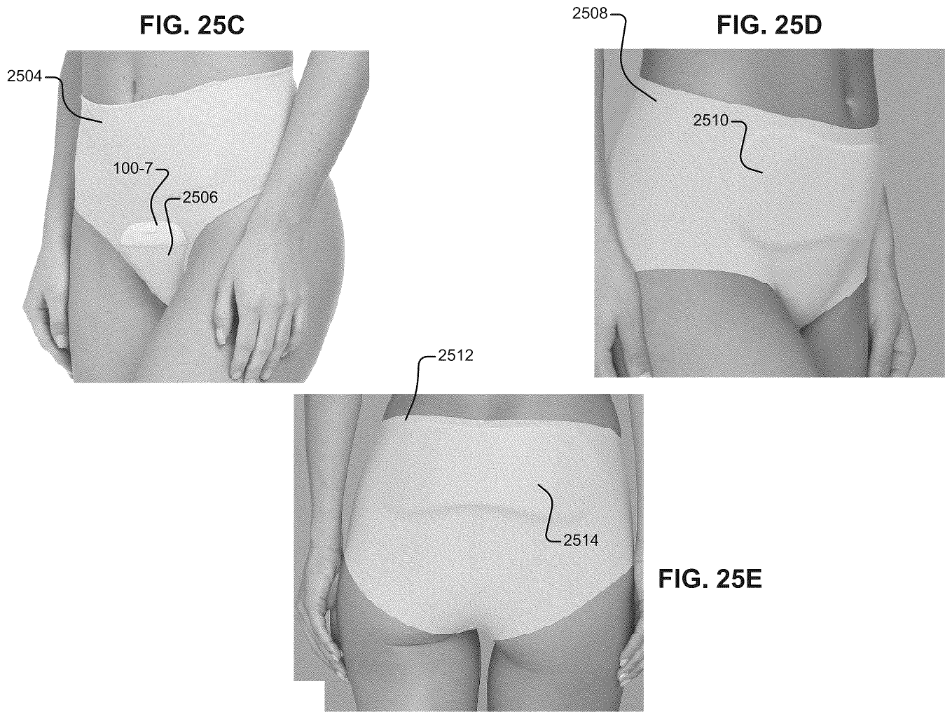

FIGS. 1A-25E illustrate features of example thermal devices 100. FIGS. 1A-1C and 18A-24B illustrate different example thermal device form factors. FIGS. 2A-2E illustrate example thermal units 200. FIGS. 3A-3G illustrate example thermal units 200 connected to other components on a package substrate 300, such as device electronics 302 and a battery 304. FIG. 4 illustrates example package substrate shapes that may include thermal units and device electronics. FIGS. 5A-5D illustrate example thermal unit fabrication steps. FIGS. 6A-6D illustrate example flexible thermal units. FIGS. 7A-7N illustrate example layouts for thermal reservoir material and insulation material. FIGS. 8A-8C illustrate example connections between the device electronics 302, battery 304, and thermal units 200. FIG. 9 illustrates communication between a thermal device 100 and a user device 104. FIG. 10 is an example functional block diagram of a thermal device. FIGS. 11A-11B illustrate example thermal profiles that may run on a thermal device 100. FIGS. 12A-12C illustrate example methods describing different thermal device modes of operation. FIG. 13 illustrates a plurality of thermal devices in communication with a remote server via a plurality of user devices. FIGS. 14A-14M illustrate heat transfer between thermal units, thermal reservoirs, and thermal bridges. FIGS. 15A-15L illustrate example thermal bridge configurations that couple the thermal unit to the user's body. FIGS. 16A-16J illustrate example thermal devices including both thermal units and heating units. FIGS. 17A-17K illustrate example GUIs on a user device that the user may interact with in order to control/monitor the thermal device. FIGS. 25A-25E illustrate example sleeves and garments that may hold the thermal device.

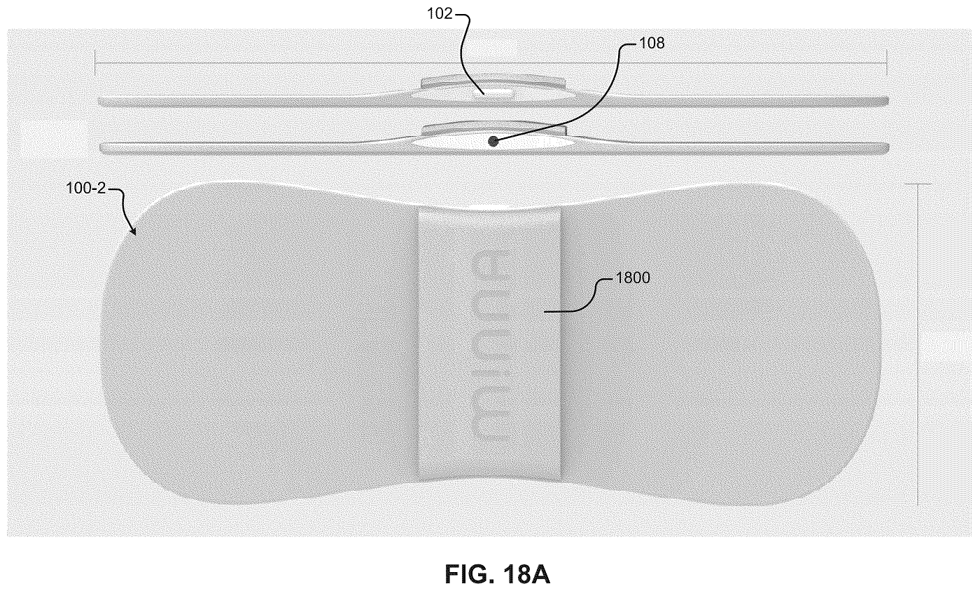

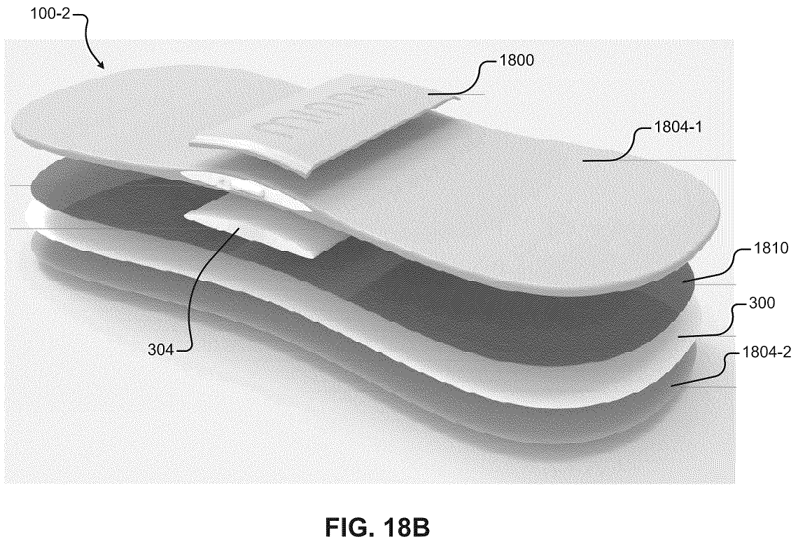

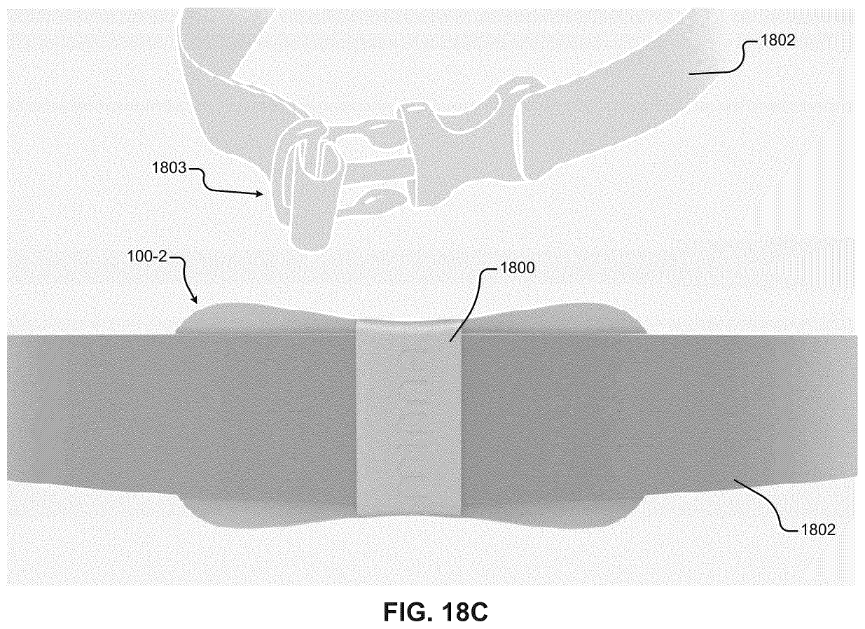

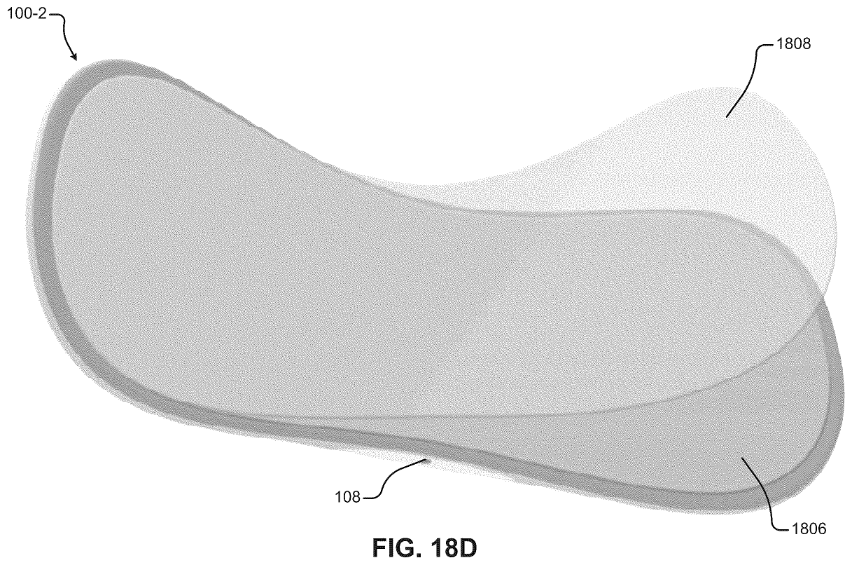

FIGS. 1A-1C illustrate a first example thermal device 100-1 (the “first thermal device 100-1”) that may include one or more thermal units 200. In some implementations, the thermal units may be separated from the user by a package substrate and/or other layers of device packaging (e.g., an encapsulation bottom cover illustrated at 1804-2 in FIG. 18B). In FIG. 1A, the thermal units may be located toward the side 106 of the thermal device 100-1 that contacts the user (e.g., the user's body/clothes). The device package may also include additional components, such as one or more thermal reservoirs and/or one or more thermal bridges. In some implementations, the additional components may be located on the side of the thermal units opposite to the user's body. Note that some thermal devices may not include the additional components (e.g., thermal reservoirs and thermal bridges). In these implementations, the thermal devices may have a thinner profile than the thermal device 100-1 illustrated in FIG. 1A (e.g., see FIGS. 18A-23A).

The first thermal device 100-1 includes a user input button 102 and power input port 108. In FIG. 1B, a power cable 110 is plugged into the power input port 108. In FIG. 1C, a user is controlling/monitoring the thermal device 100-1 using a user device 104. For example, the user may control the thermal device 100-1 to heat/cool at different intensities in different thermal zones using the GUI illustrated in FIG. 1C. The example thermal device 100-1 of FIGS. 1A-1C may have dimensions of approximately 20×10 cm, although thermal devices having different sizes and shapes may be fabricated.

FIGS. 2A-2E illustrate example thermal units 200. FIGS. 2A-2B show a generically illustrated thermal unit 200-1 that may represent any of the variety of thermal unit technologies that may be implemented in a thermal device 100 of the present disclosure. The thermal unit 200-1 of FIGS. 2A-2B includes a variety of thermal unit components that may vary, depending on how the thermal unit 200-1 is constructed. The thermal unit 200-1 may include a first thermal unit substrate 204-1, a second thermal unit substrate 204-1, and thermal unit elements 202 arranged between the first and second thermal unit substrates 204-1, 204-2 (collectively “thermal unit substrates 204). The first and second thermal unit substrates 204 may be formed from a variety of different materials, such as flexible materials or rigid materials (e.g., ceramics). The thermal unit substrates 204 may provide mechanical support for the thermal unit 200-1. The thermal unit elements 202 may be formed from a semiconductor material such as a Bismuth Telluride alloy. The thermal units 200 may also include electrical interconnects (e.g., metal interconnects) between the thermal elements 202 (e.g., that connect the thermal elements in series). In some implementations, the interconnects can be embedded in the thermal unit substrates 204 or deposited on the thermal unit substrates 204. In other implementations (e.g., FIG. 6B), the electrical interconnects can be included as a separate layer of material (e.g., a connector strip) to which the thermal unit substrates 204 are attached.

In general, the thermal unit 200 may act as a heat pump that transfers heat from one side of the thermal unit 200 to the other side of the thermal unit 200 when an electrical voltage/current is applied to the thermal unit 200. The polarity/direction of voltage/current applied to the thermal unit 200 may control the direction of heat transfer. The magnitude of the voltage/current provided to the thermal unit 200 may control the temperature difference between the thermal unit substrates 204 and/or heat flow between the thermal unit substrates 204, depending on external constraints. As described herein, device electronics (e.g., included in the device package) may control the voltage/current provided to the thermal units 200 to control whether the thermal units 200 heat or cool the user.

FIGS. 2C-2E illustrate example thermal units 200-2, 200-3. The example thermal units of FIGS. 2C-2E may represent a thermoelectric/Peltier device. FIG. 2C is a cross sectional view of the thermoelectric/Peltier device. The line drawing of FIG. 2D is a perspective view of the thermoelectric/Peltier device. The photograph of FIG. 2D is a thermoelectric/Peltier device having model number 03511-5L31-03CFL, available from Custom Thermoelectric, Inc. Bishopville, Maryland, U.S.

The devices of FIGS. 2C-2E include thermal unit elements 202 sandwiched between two thermal unit substrates 204. The thermoelectric/Peltier devices may include semiconductor thermal elements (e.g., Bismuth telluride semiconductor material) and ceramic substrates. As described herein, the thermal unit substrates 204 may be rigid or flexible. In the specific example of the thermoelectric/Peltier device of FIG. 2D, the thermal unit substrates 204 may be rigid ceramics. Although rigid ceramic substrates may be used in other thermal units of the present disclosure, in other implementations, the thermal unit substrates may include flexible materials, such as flexible polymers, flexible silicones, and/or flexible foams. Although the specific thermoelectric/Peltier device of the photograph, model 03511-5L31-03CFL, has rectangular dimensions of approximately 15 mm by 30 mm with a thickness of approximately 5.1 mm, the thermal units may be fabricated in a variety of different dimensions (e.g., 1-10 cm in length/width).

FIG. 2E illustrates an example thermal unit 200-3 including insulation material 208 between the thermal elements 202. The insulation material 208 may electrically and thermally insulate the thermal elements 202 from one another. The insulation material 208 between the thermal elements 202 may reduce thermal losses within the thermal unit 200-3 (e.g., radiative and convective heat transfer from the hot-to-cold sides inside the thermal unit 200-3). The insulation material 208 may include, but is not limited to, flexible or rigid foams, soft or hard urethanes and polymers, or silicones of varying durometer. The insulation material 208 can be electrically and thermally insulating.

FIGS. 3A-3E illustrate a variety of different arrangements of thermal device components on a package substrate 300. The package substrate 300 of FIGS. 3A-3D include device electronics 302 and a battery 304 that are centrally located on the package substrate 300. The device electronics 302 and battery 304 are offset to one side of the package substrate 300 of FIG. 3E. The device electronics 302 may be fabricated onto the package substrate 300 or separate from the package substrate 300, such as on a board separate from the package substrate 300 (e.g., a PCB) and/or fabricated onto the thermal unit substrates 204. The device electronics 302 may be secured within the package using a variety of techniques, such as with an adhesive and/or using fasteners (e.g., thread or another mechanical device). The battery 304 and device electronics 302 can be located in other locations on the package substrate 300 or in another location within the device package (e.g., on a separate PCB). The battery 304 and device electronics 302 can be colocated or located apart from one another. In some implementations, the battery 304 can be located externally on the thermal device 100 and/or detachable from the thermal device 100.

The thermal units 200 can be fabricated in a variety of different shapes and sizes. The thermal units 200 may also be arranged on the package substrate 300 in a variety of different arrangements. In FIG. 3A, the thermal units 200 have a rectangular shape and are arranged parallel to one another. Such an arrangement of thermal units 200 may allow the package substrate 300, and the whole thermal device 100, to be flexed between the thermal units 200 to the extent that the package substrate 300 is flexible. FIG. 3B illustrates a perspective view of a thermal device 100 having a similar layout to that of FIG. 3A. In FIG. 3B, the thermal device 100 is flexed (e.g., rolled) in portions of the package substrate 300 between the thermal units 200.

FIG. 3C illustrates a package substrate 300 including different shaped thermal units 200. Specifically, the package substrate 300 includes a plurality of square-shaped thermal units 200 and a plurality of triangle-shaped thermal units 200. The different shaped units may be arranged to better cover the surface of the package substrate 300, thereby providing heating/cooling to a user across more surface area of the portion of the thermal device 100 in contact with the user. The use of smaller thermal units may impart more general flexibility in different directions, even when the thermal units are rigid, because smaller units may be arranged such that more areas of flexible package substrate are available for flexing between the thermal units. In the specific example of FIG. 3C, the thermal device 100 may be flexible along portions of the flexible package substrate between any of the thermal units 200. For example, in FIG. 3C, the thermal device 100 may be bent along any line that extends between the thermal units (e.g., see bend lines of FIG. 3C).

FIG. 3D illustrates a package substrate 300 including two thermal units 200. Each of the thermal units 200 is larger than those illustrated in other figures (e.g., FIG. 3A, FIG. 3C, and FIG. 3E). The larger thermal units 200 of FIG. 3D are shaped to conform to the edges of the package substrate 300. In some cases, a larger thermal unit may provide a more consistent heating/cooling over a larger area than in the case where thermal units are separated from one another. FIG. 3E illustrates a rectangular package substrate 300 that includes device electronics 302 and a battery 304 offset towards one edge of the rectangular package substrate 300. The rectangular thermal units 200 are arranged on the rectangular package substrate 300 such that the package substrate 300 may be rolled. FIG. 3F illustrates a substrate package 300 that includes circularly arranged thermal units in which a group of peripheral thermal units surrounds a central circular thermal unit. FIG. 3G illustrates a substrate package 300 that includes arrangements of circular thermal units. In some implementations, thermal units having different shapes than those illustrated in FIGS. 3A-3G may be fabricated. For example, thermal units may be fabricated in other polygonal shapes (e.g., hexagonal shapes of FIG. 8B) or other irregular shapes.

As described herein, the thermal units may be rigid or flexible. In implementations where the thermal units are rigid, the thermal units may be arranged on a flexible package substrate that allows the overall thermal device to be flexible. Flexibility in the package substrate may allow the package substrate, and overall thermal device, to conform to the user's body during use. The entire package substrate may be formed from the same material in some cases. In other cases, the substrate may include portions that are formed from different materials.

In general, flexibility of the thermal device may be increased through the use of flexible thermal units since the thermal device may flex in areas between the thermal units and also in those areas including the thermal units. In implementations where a large portion of the package substrate is covered with a thermal unit (e.g., FIG. 3D), the thermal device can be made flexible through the use of a flexible thermal unit, whereas the thermal device may be rigid if the thermal unit is rigid.

In some implementations, a rigid package substrate can be used to impart rigidity to the overall thermal device. In other implementations, portions of the package substrate can be made rigid, while other portions may be flexible. For example, the package substrate may be made rigid in portions that include the device electronics and/or battery. A flexible portion of the package substrate may be made rigid by reinforcing a portion of the package substrate with additional material and/or different material (e.g., stiffening material and/or stiffening structures). In one specific example, the package substrate 300 of FIG. 3A may be formed from a single piece of flexible material that is made rigid under the device electronics 302 and battery 304. In this specific example, the portions including thermal units may be flexed while the portion of the package substrate under the device electronics and battery remain rigid.

In some implementations, the thermal device 100 may be configured such that the thermal units 200 are controlled independently from one another. In these implementations, each thermal unit 200 may be electrically coupled to the device electronics 302 using separate electrical connectors. The device electronics 302 may provide power to each of the thermal units 200 independently of other thermal units 200 via the separate electrical connectors. The thermal device 100 may provide more granular heating/cooling in implementations where the thermal units are independently controlled.

In some implementations, the thermal device 100 may be configured such that the device electronics 302 controls groups of thermal units 200 together. For example, a group of thermal units 200 may be electrically coupled to one another so that the device electronics 302 controls the group of thermal units 200 together (e.g., via a single pair of wires). In these implementations, the control of heating/cooling may be less granular than independent control of the thermal units 200, however, the wiring layout and control scheme may be simplified in some respects.

As described herein, the overall shape and flexibility of the thermal device 100 may be fabricated based on where the thermal device 100 is to be used on the body. Additionally, the size, shape, and overall coverage of the thermal units 200 may be selectable. Furthermore, the control techniques for the different thermal units 200 (e.g., individual/grouped) may be selected in order to control the granularity of heating/cooling. Accordingly, a variety of different thermal devices 100 can be fabricated for a variety of different uses according to the present disclosure. For example, thermal devices 100 may be fabricated to conform to different parts of a user's body, such as a user's back, wrist, legs, perineum region, etc. Additionally, a thermal device 100 may be fabricated in an eye-mask shape for migraine headache relief, a knee wrap for knee pain, or neck/shoulder wrap.

FIG. 4 illustrates a variety of different example package substrate shapes 400 and features, such as rectangular package substrates 400-1, a package substrate 400-2 including cutouts, package substrates 400-3 including protrusions 403 (e.g., lobes), and a package substrate 400-4 including strips 405. Although not illustrated, the package substrates of FIG. 4 may include thermal units. For example, the thermal units may be attached to the substrate or fabricated onto the substrate. The thermal units may be attached to the package substrate using adhesives, mechanical fastening, mechanical constraint within a pocket/region of the substrate, ultrasonic or heat welding, and other techniques.

The substrates (e.g., package substrates and/or thermal unit substrates) can be formed from any material that is tolerant to the levels of heat/cold generated by the thermal units. In some implementations, the substrates may also be tolerant to heat generated during processing steps used to fabricate the thermal device, although some substrates may not be exposed to elevated temperatures during fabrication, depending on how the thermal device is fabricated. Example materials may include, but are not limited to, polyester, polyimide, and silicone. In some implementations, the substrates may include a single layer of material. In other implementations, the substrates may include multiple layers of material that are bonded to one another or otherwise joined together.

FIGS. 5A-5D illustrate example fabrication steps for fabricating thermal units 200-4, 200-5. FIG. 5A illustrates thermal elements 202 being placed onto an example thermal unit substrate 500. The thermal unit substrate 500 may be either a rigid substrate or a flexible substrate. The thermal unit substrate 500 includes portions for receiving the thermal elements 202. The portions that receive the thermal elements 202 may include electrical conductors 501 (e.g., metal) that electrically couples adjacent thermal elements. In FIGS. 5A-5D, the substrates 500, 502 may be fabricated from flexible printed circuit boards with integrated electrical traces that route current through the thermal elements 202.

The thermal unit substrate 500 of FIG. 5B includes a plurality of thermal elements 202. The thermal elements 202 are electrically coupled to one another via connector strips 503, which may be rigid or flexible. The connector strips 503 may include electrical conductors that electrically couple the thermal elements 202. The connector strips 503 can be connected to the thermal elements 202 prior to attachment to the thermal unit substrate 500 or after the thermal elements 202 are attached to the thermal unit substrate 500. The connector strips 503 may be fabricated from flexible circuit board material (e.g., a polyimide material) that includes pads connected by conductive traces. In some implementations, the connector strips 503 may include a metallic material (e.g., copper) or be formed completely from a metallic material. Instead of using individual connector strips, in some implementations, the thermal elements 202 may be connected using a single continuous sheet of flexible circuit board material including traces that electrically connect the thermal elements 202 of the thermal unit 200. Note that the thermal unit substrate 500 also includes sensors 504 (e.g., temperature sensors) in addition to the thermal elements 202.

The thermal unit can have electrical connectors that electrically couple the thermal unit to the device electronics. In some implementations, the electrical connectors can include metal traces on the thermal unit substrate material. Such connectors can be included on a flexible strip of substrate material (e.g., at 505) that is continuous with the portion of the thermal unit substrate including the thermal elements, as illustrated in FIGS. 5A-5D. In other implementations, the thermal unit substrates can include electrical contacts (e.g., metal pads) that can connect to wires (e.g., be soldered to wires), which are in turn connected to the device electronics.

FIGS. 5C-5D illustrate fabrication of another thermal unit 200-5. In FIG. 5C, the thermal elements 202 are being placed onto the thermal unit substrate 502-1. The thermal unit substrate 502-1 of FIG. 5C includes a top cover 502-2 that includes electrical contacts 506 for electrically coupling the thermal elements 202 to one another (e.g., in a similar manner as the connector strips 503 in FIG. 5B). The top cover 502-2 may be folded over on top of the bottom thermal unit substrate 502-1 at the flexible ribbon portion 507 between the top cover 502-2 and the bottom thermal unit substrate 502-1. The fabricated thermal unit 200-5 is illustrated in FIG. 5D. Note that the top cover 502-2 includes a sensor 508 (e.g., a temperature sensor).

FIGS. 6A-6D illustrate example flexible thermal units 200-6, 200-7, 200-8, 200-9. In FIG. 6A, the thermal unit 200-6 includes thermal elements 202 that are connected to one another by flexible connector strips 600. The flexible connector strips 600 allow the thermal unit 200-6 to be flexed in multiple directions, as illustrated in FIG. 6A. The thermal unit 200-6 includes electrical contacts 602 on flexible connector strips that may be connected to the device electronics. FIG. 6D illustrates another example thermal unit 200-9 including connector strips 604. The connector strips 604 of FIG. 6D span more than two thermal elements 202. Although the connector strips 604 of FIG. 6D are attached to additional thermal elements relative to FIG. 6A, the connector strips 604 of FIG. 6D may electrically couple the thermal elements 202 in the same manner as FIG. 6A. The longer strips 604 may simplify fabrication of the thermal unit 200-9 (e.g., using fewer parts) while maintaining mechanical flexibility.

The thermal unit 200-7 of FIG. 6B includes flexible thermal unit substrates 606 on both sides of the thermal elements 202. The flexible thermal unit substrates 606 may provide additional support for the thermal unit 200-7. In some implementations, the thermal unit substrates 606 of FIG. 6B may be attached to a package substrate. In other implementations, the thermal elements 202 may be attached directly to the package substrate, which may include portions for receiving the thermal elements 202. In these implementations, the package substrate may include electrical connections that electrically couple the thermal elements 202 to one another.

FIG. 6C illustrates an example thermal unit 200-8 having material 608 deposited between the individual thermal elements 202. The material 608 may be a flexible insulation material, as illustrated in FIG. 6C. The flexible insulation material 608 may serve to prevent contact between neighboring thermal elements 202, thermally insulate the cold/hot sides during use, and promote rebound to a pre-defined bend configuration. The flexible insulation material 608 may include flexible silicone foams or solids, flexible urethane foams or solids, polymers of varying durometer, and other elastomeric materials.

In some implementations, the thermal elements and device electronics can be fabricated onto a single package substrate (e.g., 300) that supports the device electronics and the thermal elements. In these implementations, the package substrate may act as a thermal unit substrate. In some cases, during fabrication, the package substrate can include additional portions that are folded back over the thermal elements to act as another thermal unit substrate (e.g., similar to FIGS. 5C-5D), thereby forming one or more thermal units from a single sheet of package substrate material. The thermal elements may be arranged to make thermal zones of any geometry (e.g., arranged on the substrates of FIG. 4).

FIGS. 7A-7N illustrate example thermal units that interface with various different thermal reservoirs (e.g., 700-1, 700-2, . . . , 700-8), generally referred to herein as a “thermal reservoir 700.” A thermal reservoir 700 may act as an energy storage material that may sink or source energy. For example, the thermal reservoir may act as a heat sink that can receive energy from the hot side of one or more thermal units 200. As another example, the thermal reservoir may act as a heat source that can transfer energy to the cold side of one or more thermal units. Example thermal reservoir materials may include phase-change materials, which may be designed to change from solid to liquid at a pre-determined temperature near the average human body temperature. Example phase-change materials may include paraffin, lipids, salt hydrates, and other organic and inorganic materials. An example phase-change material is PCM-0M37P manufactured under the savENRG brand of Arden, NC USA. This material may change from a solid to a liquid at approximately 37 degrees Celsius, allowing it to absorb a substantial amount of heat while remaining very close to 37 degrees Celsius. Non-phase-change materials may also be used as a thermal reservoir. Gels and liquids that have a high heat capacity may allow for rapid heat absorption and distribution while maintaining flexibility of the thermal reservoir material. Solid materials may also be used as a thermal reservoir. Solid materials such as brass, bronze, and copper may also have a combination of high density, moderate heat capacity, and high thermal conductivity. Combinations of liquid and solid materials within a thermal reservoir, as well as liquid and phase-change materials may be used to optimize the density, thermal conductivity, and heat capacity. Balancing of these material properties may allow for maximal heat storage.

In some implementations, the thermal reservoir 700 may be in direct contact with the thermal unit substrate. In other implementations, other materials may be included between the interface of the thermal reservoir 700 and the thermal unit, such as a thermal grease or other thermally conductive materials. In some implementations, the thermal reservoir material may be deposited onto the thermal unit substrate such that the thermal reservoir material is adhered to the thermal unit substrate. In other implementations, the thermal reservoir material may be in contact with the thermal unit substrate, but not bonded to the thermal unit substrate. In some implementations, the thermal reservoir material may be encapsulated inside a containment material, such as a thin plastic film. This may be done when using a thermal reservoir material that is a gel, liquid, or phase-change material in order to avoid leakage and flow away from the thermal unit. Furthermore, within the containment material (e.g., a containment cell or baffle), there may be a plurality of reservoir materials. In one implementation, a thermal reservoir 700 may include a phase-change material accompanied by water within a containment material. In one specific implementation of this type, a waxy paraffin-based phase-change material may melt as it absorbs heat but will not dissolve within the water. In this implementation, the water may serve to ensure good thermal contact between the thermal unit and the phase-change material. As the material cools, the waxy substance may return to a solid phase, leaving the water in its liquid phase.

In some implementations, the thermal reservoir material may be deposited over individual thermal units (e.g., FIGS. 7A-7B). In other implementations, a continuous thermal reservoir material may be included over top of a plurality of thermal units (e.g., FIG. 7C). In some implementations, a single thermal reservoir material may be deposited (e.g., in a layer) relatively evenly over the thermal units. In other implementations, multiple layers of thermal reservoir material may be deposited over the thermal units. In some implementations, the thermal reservoir material may be included as distinct depositions over top of individual thermal elements (e.g., FIGS. 7F-7G). Additional thermal reservoir material may be deposited over top of the distinct depositions (e.g., FIGS. 7H-7I).

In some implementations, the thermal reservoir material may be included inside the device package (e.g., not readily removable). In other implementations, the thermal reservoir material may be inserted and removed from the thermal device (e.g., as a removable thermal reservoir package). A removable thermal reservoir material may include a flexible gel pack or assembly of solid materials. In other implementations, the thermal reservoir material may be a liquid, such as water, which may be emptied and refilled by the user. In these implementations, the thermal reservoir material may be removed and replaced.

In some implementations, a thermal device 100 may use an external thermal reservoir (e.g., to dissipate heat). For example, in implementations where the thermal device 100 is waterproof, a user may dip the thermal device into an external thermal reservoir (e.g., a water bath) or apply an external thermal reservoir (e.g., a water or ice pack) during use. In one specific example, if the user is wearing a thermal device 100 around their ankle, they may dip their foot and ankle into a water bath to dissipate heat from the thermal device 100. In another example, a user may use their body as a thermal reservoir. For example, a user may place their hand over the thermal device 100 and/or sandwich the thermal device 100 between two body parts (e.g., the upper arm and the chest).

FIGS. 7A-7B illustrate example thermal reservoirs 700-1, 700-2 deposited on thermal units 200-1. The thermal reservoirs 700-1, 700-2 of FIGS. 7A-7B have different geometries. The thermal reservoir 700-2 of FIG. 7B is smaller than the thermal reservoir 700-1 of FIG. 7A and has a lower profile than that of the thermal reservoir 700-1 of FIG. 7A. The thermal reservoir 700-1 of FIG. 7A is mounded over the thermal unit 200-1, whereas the low profile thermal reservoir 700-2 of FIG. 7B is more conformal to the underlying thermal unit substrate.

The example thermal reservoir 700-3 of FIG. 7C spans across multiple thermal units 200-1. As such, the single thermal reservoir 700-3 can transfer heat with the multiple thermal units. Although only two thermal units are illustrated in FIG. 7C, a single thermal reservoir may span over more than two thermal units. In some implementations, the same thermal reservoir can span over all the thermal units in the thermal device.

FIG. 7D illustrates a perspective view of a single thermal reservoir 700-4 deposited over top of a single thermal unit 200-8. Note that the thermal reservoir 700-4 may flex along with the underlying thermal unit 200-8. FIG. 7E illustrates a cross-sectional view of the device in FIG. D. The thermal unit 200-8 of FIG. 7E includes an insulation material 608 between the thermal elements 202.

FIGS. 7F-7G illustrate an example thermal unit 200-8 including multiple separate thermal reservoirs 700-5. The distinct thermal reservoirs 700-5 each correspond to a different thermal element 202 or group of thermal elements. For example, the thermal reservoirs 700-5 may be deposited over single thermal elements or groups of thermal elements. The thermal reservoirs 700-5 may be bonded to the thermal unit substrate 606. In one implementation, the thermal reservoir 700-5 may comprise a cell made from a thin film of plastic and contain a mixture of phase-change material and a liquid, such as water. In this implementation, the thermal reservoir cell may be directly adhered, welded, fused, or otherwise bonded onto the thermal unit substrate 606.

FIGS. 7H-7I illustrate an example thermal unit 200-8 including multiple separate thermal reservoirs 700-5, as described with respect to FIGS. 7F-7G. Additionally, the thermal unit 208 of FIGS. 7H-7I includes an additional thermal reservoir material 700-6 deposited over the separate thermal reservoirs 700-5. The additional thermal reservoir material 700-6 may provide additional energy storage. In some implementations, the additional thermal reservoir material 700-6 may include a gel and/or liquid to provide flexibility. Although two thermal reservoir materials 700-5, 700-6 are illustrated in FIGS. 7H-7I, in other implementations, further thermal reservoir materials may be added. In some implementations, a thermal unit may include multiple flattened layers of different thermal reservoir materials (e.g., multiple layers without the distinct materials deposited in FIGS. 7H-7I).

FIGS. 7J-7K illustrate cross sections of two example thermal devices. In FIG. 7J, each thermal unit 200-1 includes a separate thermal reservoir 700-2. The two thermal units 200-1 are attached to a package substrate 702 that interfaces with the user's body. In some implementations, the package substrate 702 may be made from a material with high thermal conductivity, such as copper mesh or an elastomer that has been doped to promote thermal conductivity. The thermal devices also include additional device packaging over top of the thermal reservoirs (e.g., a top encapsulation layer 708). The top encapsulation layer 708 may be formed from material such as fabric (e.g., cloth), polymer, elastomer, or other material. In some implementations, the top encapsulation layer 708 may be omitted. The thermal device of FIG. 7K has a similar structure to that of FIG. 7J, however, the thermal device of FIG. 7K includes an insulating material 704 between the thermal units 200-1. The insulating material 704 may include a flexible foam material. Additionally, the thermal device of FIG. 7K includes an encapsulation bottom layer 706. The encapsulation bottom layer 706 may be made from a flexible material, such as a silicone or a fabric material, in addition to various other materials.

FIG. 7L illustrates another example thermal reservoir 700-7. The thermal reservoir 700-7 of FIG. 7L includes a thermal reservoir casing 710 that may be capped with a thermal reservoir cap 712. The thermal reservoir casing 710 defines a cavity 713 (i.e., a repository/reservoir) for including additional thermal reservoir material (e.g., a phase-change material). The cavity 713 includes a plurality of pillars 714 that increase the area for heat exchange with the thermal reservoir material. The thermal reservoir cap 712 seals the thermal reservoir material in the cavity 713. The thermal reservoir casing 710 may be fabricated from a variety of materials. In some implementations, the thermal reservoir casing 710 may be fabricated using stamping, casting, injection molding, or extrusion. The sealed thermal reservoir casing can be used as a thermal reservoir for the thermal unit 200 illustrated in FIG. 7L. Although a round thermal reservoir including a cavity is illustrated in FIG. 7L, other geometries of thermal reservoirs including cavities may be fabricated. The thermal reservoir casing 710 may be formed from materials having good thermal conductivity, such as a metal (e.g., aluminum) and/or polymer.

FIGS. 7M-7N illustrate additional examples of how thermal reservoir material may be included in a thermal device. In FIGS. 7M-7N, the thermal unit is sandwiched between two thermally conductive layers 716-1, 716-2. The bottom layer 716-2 is a body contact layer. The top thermally conductive layer 716-1 is connected to thermal reservoir material 700-8. The two sides of the thermal unit are insulated from one another. Accordingly, the two thermally conductive layers 716-1, 716-2 are insulated from one another. In FIG. 7M, the insulation may be provided by separation (e.g., an air gap) between the thermal reservoirs 700-8 and the body contact layer 716-2. In FIG. 7N, insulation material 718 is added between the thermal reservoirs 700-8 and the body contact layer 716-2. The arrangement of thermal reservoir material 700-8 in FIGS. 7M-7N may reduce the overall thickness of the thermal device relative to implementations in which the thermal reservoir material is located above the thermal units (e.g., see FIG. 7J).

The device electronics 302 control the amount of heating/cooling provided by a thermal unit 200 by controlling the delivery of power to the thermal unit 200. For example, the device electronics 302 may control power delivered to a thermal unit 200 by controlling the voltage applied across the thermal unit 200 (i.e., between two contacts). As another example, the device electronics 302 may control the power delivered to a thermal unit 200 by controlling the current through the thermal unit 200. In some implementations, the thermal device 100 may include maximum power delivery values, such as a threshold power/current/voltage level at which the thermal device 100 may limit the delivery of power to one or more thermal units 200.

The layout of the thermal units 200 defines the thermal zones. In some implementations, the shape of the package substrates can be configured to match the thermal zones. For example, with respect to the package substrate 400-3 of FIG. 4 that includes a plurality of lobes 403, each of the lobes 403 can include one or more thermal units 200. In this example, each of the lobes 403 may include a thermal zone.

In some implementations, the package substrate (e.g., 300, 400) may include an adhesive layer (not illustrated). The adhesive layer can attach to the package substrate on one surface and adhere to the user's skin on the other surface. The skin adhesive layer may include, but is not limited to, silicone gels, acrylic adhesives, polyurethane gels, and hydrogels. The adhesive layer can include a removable cover layer that may be peeled from the adhesive layer to expose the adhesive layer. The removable cover layer may be a smooth layer that adheres to the underlying adhesive but does not adhere to the user. In some implementations (e.g., FIG. 18D), the adhesive layer 1806 and removable cover layer 1808 may be attached to the outside of the device package instead of the package substrate (e.g., if the package substrate is included under additional layers). In some implementations, the adhesive layer may be removable. For example, the adhesive layer may include an adhesive or other type of attachment for connecting to the package substrate or other portions of the device package. In some implementations, the thermal device may include additional adhesive layers (not shown) used in construction of the thermal device, such as adhesive layers that adhere different package components to one another.

The device electronics 302 can control heating/cooling based on a thermal profile, user input, and/or sensor data (e.g., in the manual/automatic/mixed mode). The device electronics 302 may also perform a variety of other functions described herein. For example, the device electronics 302 can provide communication with the user device 104, control charging of the battery 304, and control interactions with user interface devices (e.g., user input button 102).

The device electronics 302 can be mounted in a variety of different locations. In some implementations, the device electronics 302 can be mounted (e.g., soldered) to the package substrate (e.g., see FIG. 3B). In FIG. 3B, the device electronics 302 are included on a portion of the package substrate 300 that is more rigid than the rest of the package substrate 300. In other implementations, the device electronics 302 may be attached to a flexible portion of the package substrate 300.

Although the device electronics 302 can be mounted to a package substrate including thermal units, in some implementations, at least a portion of the device electronics 302 can be mounted in another location. For example, with respect to FIGS. 8A-8C, the device electronics 302 can be mounted to a printed circuit board (PCB) 800 that is external to the package substrate, but included in the device package. In these implementations, the PCB 800 including the device electronics 302 can be electrically coupled to the thermal units 200 (e.g., via wires that connect the device electronics to the thermal units).

In some implementations, an external PCB 800 can be wired (e.g., permanently) to the thermal units 200. For example, the external PCB 800 can be soldered or otherwise connected to the thermal units 200 (e.g., via wires). In other implementations, as illustrated in FIGS. 8A-8C, the thermal device can include a thermal unit connector 806 that can electrically couple the external PCB 800 to the thermal units 200. The thermal unit connector 806 can include two connection components 806-1, 806-2 that can be disconnected from one another so that the external PCB 800 and the thermal units 200 can be disconnected from one another. The thermal unit connector 806 can include an electronics side 806-1 and a thermal unit side 806-2. The two sides of the connector 806 can be connected to electrically couple the device electronics 302 and the thermal units 200. The illustrated connector 806 is a low-profile connector, such as a Molex 36877-0004 connector. The connector 806 may have a positive-latching connector design so that the connector 806 does not become detached during use. Additionally, the connector may be water-proof to allow for easy cleaning or moisture exposure during use. In some implementations, the PCB 800 can be connected to the thermal units 200 with other types of detachable connectors than those illustrated. For example, the external PCB 800 may include a socket into which the thermal unit connectors on the package substrate can be inserted, such as a Universal Serial Bus (USB) connection or other low profile power connector. As an additional example, the package substrate may include a socket into which the external PCB wires/connectors can be inserted.

In implementations where the device electronics 302 are detachable from the thermal unit(s) (e.g., via the thermal unit connector 806), different package substrates having different arrangements of thermal units (e.g., layout/number of thermal units) and sensors may be interchangeable with the same device electronics 302. In other cases, a new package substrate with thermal units having the same arrangements as the old package substrate could be swapped out (e.g., in the case the old thermal units are broken or worn out).

FIGS. 8B-8C illustrate how different package substrates 802, 804 having different thermal unit arrangements can be connected to the device electronics 302 via the thermal unit connector 806. In FIG. 8B, the device electronics 302 can connect to a plurality of thermal units 200 and a temperature sensor 808 included on the substrate. In FIG. 8B, the device electronics 302 can deliver power to the thermal units 200 and also determine the temperature indicated by the temperature sensor 808.

In FIG. 8C, the device electronics 302 are connected to a package substrate 804 having a different number and arrangement of thermal units 200. Although the arrangements of thermal units and sensors are different from FIG. 8B, in some implementations, the same device electronics 302 may be configured to operate the thermal units 200 and temperature sensor 808 of FIGS. 8B-8C. For example, the device electronics 302 can be configured to deliver power to the thermal units 200 of FIG. 8B and determine the temperature indicated by the temperature sensor 808 in FIG. 8B. The device electronics 302 can then reconfigure to deliver power to the thermal units 200 of FIG. 8C.

In some implementations, the device electronics 302 can deliver power to the thermal units 200 and measure temperature using the same circuits. For example, if the temperature sensor 808 is a resistive temperature sensor (e.g., a thermistor or resistance temperature detector), the device electronics 302 may include circuits that deliver power to the sensor 808 in a manner similar to the thermal units 200, determine the resistance of the sensor 808, and determine temperature based on the determined resistance. In other implementations, the device electronics 302 may include additional components that interface with the temperature sensor 808, such as circuits that interface with a thermocouple or a digital temperature sensor. The device electronics 302 may include switches (e.g., discrete switches and/or switches included on a microcontroller) that may be used to reconfigure the functionality for each of the electrical contacts provided by the device electronics 302 (e.g., pinouts/wires). The device electronics 302 may be configured to operate while connected to a different number of connections than illustrated.

As described with respect to FIGS. 8B-8C, the device electronics 302 can be configured (e.g., using switches) to couple to sensors and/or thermal units using the same electrical contacts. In some implementations, the device electronics 302 may be configured to operate with a variety of different thermal units having a different number of contacts, different arrangements, and/or different types of sensors. The device electronics 302 may determine how to operate with different thermal units in a variety of different ways. In some implementations, a user may manually configure the device electronics 302 (e.g., using a GUI on the user device 104) to operate with a specific arrangement of thermal units. For example, the user may enter a model number of the package substrate including the thermal units into the GUI that indicates to the user device 104 and/or thermal device 100 how to configure the device electronics 302 for operating the specific thermal units and sensors. In some implementations, the device electronics 302 may automatically detect the specific package substrate and/or thermal units attached to the device electronics 302 and then correctly operate the thermal units. The device electronics 302 may automatically detect the package substrate and/or thermal units in a variety of ways, such as via applying test voltage/current to determine the thermal unit arrangement and whether a sensor is attached. In some cases, a package substrate and/or thermal units may include an identification circuit (e.g., a ROM) that indicates details of the package substrate and/or thermal units to the device electronics 302, such as the number of thermal units, the arrangement of thermal units, and the number/arrangement of sensors. The device electronics 302 may determine the configuration of the package substrate and/or thermal units and how to operate the package substrate and/or thermal units based on communication with the identification circuit (e.g., by reading the ROM). In some implementations, the device electronics 302 may also operate the thermal units based on communication with the battery (e.g., an external battery that may be swapped for another battery). For example, the device electronics 302 may identify battery parameters, such as storage capacity, charge parameters, etc., and control the thermal units based on the identified parameters.

FIG. 9 illustrates a thermal device 100 in communication with a user device 104 (e.g., a cell phone). The device electronics 302 can include wireless/wired communication technology that communicates with the user device 104. As described herein with respect to FIG. 13, the user device 104 can communicate with remote server 1302 via a network 1304, such as the internet. The user device 104 can also provide a variety of functionality with respect to the thermal device 100. In some implementations, the user device 104 may generate a GUI (e.g., FIGS. 17A-17K) that the user may use to perform a variety of different operations with respect to the thermal device 100. For example, the user may interact with the GUI to control heating/cooling. In some examples, the user may interact with GUI element controls to control heating/cooling. In other examples, the user may select a thermal profile and upload the thermal profile to the thermal device 100 using the GUI. The user may select a profile on the thermal device 100 to run, select a thermal profile from the user device 104 to load onto the thermal device 100, and/or retrieve a thermal profile from a remote server 1302 to run on the thermal device 100. The user may also monitor various heating/cooling parameters, such as the battery status, the currently running thermal profile (e.g., a thermal map), and the remaining time for which the thermal device 100 may run the thermal profile. Additional features of the user device 104, thermal device 104, and aspects of communication between the devices 100, 104 are described herein.

FIG. 10 is a functional block diagram of an example thermal device 1000. The various modules represent functionality (e.g., circuits and other components) included in the thermal devices 100, 1000 of the present disclosure. Modules of the present disclosure may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits, etc.). Memory may include any volatile, non-volatile, magnetic, or electrical media, such as a random access memory (RAM), read-only memory (ROM), non-volatile RAM (NVRAM), electrically-erasable programmable ROM (EEPROM), Flash memory, or any other memory device.

Furthermore, memory may include instructions that, when executed by one or more processing circuits, cause the modules to perform various functions attributed to the modules herein. The device electronics of the thermal devices described herein are only example device electronics. As such, the types of electronic components used to implement the device electronics may vary based on design considerations.

The functions attributed to the modules herein may be embodied as one or more processors, hardware, firmware, software, or any combination thereof. Depiction of different features as modules is intended to highlight different functional aspects and does not necessarily imply that such modules must be realized by separate hardware or software components. Rather, functionality associated with one or more modules may be performed by separate hardware or software components, or integrated within common or separate hardware or software components.

The thermal device 1000 includes a processing module 1002 (e.g., a processor and/or microcontroller), a communication module 1004, an interface module 1006, a power module 1008, a thermal control module 1010, and a temperature sensing module 1012. The thermal device 1000 may also include a battery 1014, thermal units 1016-1, 1016-2, . . . , 1016-N, and one or more sensors 1018 (e.g., a temperature sensor). The processing module 1002 communicates with the modules included in the thermal device 1000. For example, the processing module 1002 may transmit/receive data to/from the modules and other components of the thermal device 1000. As described herein, the modules may be implemented by various circuit components. Accordingly, the modules may also be referred to as circuits (e.g., a communication circuit, temperature sensing circuit, thermal control circuit, interface circuit, and power circuit).

The processing module may communicate with the memory 1020. The memory 1020 may include computer-readable instructions that, when executed by the processing module 1002, cause the processing module 1002 to perform the various functions attributed to the processing module 1002 herein. The memory 1020 may include any volatile, non-volatile, magnetic, or electrical media, such as RAM, ROM, NVRAM, EEPROM, Flash memory, or any other digital media. In some implementations, the processing module 1002 may include a microcontroller which may include additional features associated with other modules, such as an integrated Bluetooth Low Energy transceiver.

The temperature sensing module 1012 is electrically coupled to the temperature sensor 1018. The temperature sensor 1018 indicates the temperature in the area in which the temperature sensor 1018 is located. The temperature sensing module 1012 may determine the temperature in the location of the temperature sensor 1018. In some implementations, the temperature sensor 1018 may generate a temperature signal that indicates the temperature in the area. For example, the temperature sensor 1018 may generate a digital signal that the temperature sensing module 1012 may use to determine the temperature. As another example, if the temperature sensor 1018 is a passive thermistor, the temperature sensing module 1012 may measure a current/voltage generated by the temperature sensor 1018 and determine the temperature based on the measured current/voltage.

The interface devices 1022 may include user-feedback devices and/or user input devices. For example, user-feedback devices may include, but are not limited to, a display (e.g., a touchscreen display), vibration devices, lighting devices (e.g., LEDs), and a speaker. The interface module 1006 can control the user-feedback devices. For example, the interface module 1006 may include display control/driver circuits, vibration control circuits, LED control circuits, speaker control circuits, and/or other control circuits. In some implementations, the processing module 1002 may control the interface devices 1022 via the interface module 1006. For example, the processing module 1002 may generate control signals that the interface module 1006 uses to control the interface devices 1022. For example, the interface module 1006 may include circuits that deliver power/data to the display/vibration/lighting devices, while the processing module 1002 controls the delivery of power/data to the display/vibration/lighting devices.

Example user input devices include, but are not limited to, buttons (e.g., manual buttons and/or capacitive touch sensors), switches, and a touchscreen. The interface module 1006 may include circuits for receiving user input signals from the user input devices. The processing module 1002 may receive the user input signals from the interface module 1006 and take a variety of actions based on the user input signals. For example, the processing module 1002 may detect a user pushing an on/off button and then power up the thermal device 1000 in response to detection of the press. As another example, the processing module 1002 may detect a user pressing a thermal control button (e.g., +/− buttons) and then increment/decrement the amount of heating/cooling provided by the thermal units 1016 based on detection of the press.

The communication module 1004 can include circuits that provide wired and/or wireless communication with the user device 104. In some implementations, the communication module 1004 can include wired communication circuits, such as USB communication circuits. In some implementations, the communication module 1004 can include wireless communication circuits, such as Bluetooth circuits and/or WiFi circuits.

Using the communication module 1004, the thermal device 1000 and the user device 104 can communicate with each other. The processing module 1002 can transmit/receive data to/from the user device 104 via the communication module 1004. Example data may include thermal profiles and other information requests, such as status updates (e.g., charging status, battery charge level, and/or thermal device configuration settings). The processing module 1002 can also receive instructions/commands from the user device 104, such as instructions to increase/decrease heating/cooling. In some implementations, the processing module 1002 (e.g., a microcontroller) may include circuits that provide wired/wireless communication (e.g., USB/Bluetooth). In some implementations, the user device 104 can transfer update data to the thermal device 1000 to update the software/firmware of the thermal device.

The thermal device 1000 may include a battery 1014 (e.g., a rechargeable or non-rechargeable battery). An example battery may include a Lithium-Ion or Lithium-Polymer type battery, although a variety of battery options are possible. A power source (e.g., a wall adapter power cord or USB power plug) can be plugged into the power input port 108 of the thermal device 1000 to charge the battery 1014. In some implementations, the thermal device 1000 may not include a battery. Instead, the thermal device 1000 may be powered via the power input port 108. The thermal device 1000 includes a power module 1008 that may control charging of the battery 1014, regulate voltage(s) of the device electronics 302, regulate power output to the device electronics 302, and monitor the state of charge of the battery 1014. In some implementations, the battery itself may contain a protection circuit module (PCM) that protects the battery from high current discharge, over voltage during charging, and under voltage during discharge. In some implementations, the power module 1008 may include circuits configured to modulate the voltage and current into the battery 1014 during charging. For example, the power module 1008 may include a Microchip MCP73832 charge control IC and supporting passive components. The power module 1008 may also include electro-static discharge (ESD) protection.

In some implementations, the power module 1008 may control charging of the thermal device 1000 from the user device 104. For example, the thermal device 1000 may draw power from the user device 104 (e.g., a laptop or tablet), which may allow the thermal device 1000 to run longer. In some implementations, the power module 1008 may control charging of the user device 104 or other equipment from the thermal device 1000. For example, the thermal device 1000 can deliver power to the user device 104 (e.g., a phone or tablet) to extend the battery life of the user device 104, which the user may be using to control the thermal device 1000. In some cases, if the user device 104 is in communication with the thermal device 1000 and the battery is running low on the user device 104, the user device 104 may prompt the user to plug into the thermal device 1000 in order to charge the battery of the user device 104. In other cases, if the user device 104 is in communication with the thermal device 1000 and the battery 1014 is running low on the thermal device 1000, the thermal device 1000 may prompt the user to plug the thermal device 1000 into the user device 104 in order to charge the battery 1014 of the thermal device 1000 (e.g., prompt via a GUI on the user device 104).

The processing module 1002 along with the thermal control module 1010 can control the amount of heating/cooling provided by the thermal units 1016. For example, the thermal control module 1010 can include electronics that control the amount/polarity of power delivered to the thermal units 1016. In one example, the thermal control module 1010 can include electronics that switch on/off the delivery of power to the individual thermal units 1016. As another example, the thermal control module 1010 can include electronics that can incrementally adjust the power delivery to the thermal units 1016 (e.g., adjust current and/or voltage).

The processing module 1002 may control the thermal control module 1010 to deliver power to the thermal units 1016 according to user input and/or a thermal profile. In some implementations, the thermal control module 1010 may include metal-oxide semiconductor field-effect transistor devices (MOSFETs) (e.g., power MOSFETs) that are controlled by a gate voltage generated by the processing module 1002 (e.g., a microcontroller). In implementations where MOSFET devices are used to control current through the thermal units 1016, the MOSFETs may be controlled via pulse-width modulation (PWM) signals or on/off commands generated by the processing module 1002 (e.g., microcontroller). In another implementation, the thermal units 1016 may receive power from a variable voltage power supply within the device (instead of binary on/off control). In some implementations, the thermal control module 1010 may include circuits for changing polarity of the voltage delivered to the thermal units 1016 (e.g., an H-bridge circuit).

The processing module 1002 may control the thermal control module 1010 in a variety of different modes (e.g., a manual mode, automatic mode, and mixed mode). In the manual mode, the processing module 1002 may control the thermal control module 1010 to deliver power based on user input received via the user input devices on the thermal device 1000 and/or based on user input received from the user device 104 (e.g., via wireless communication). In the automatic mode, the processing module 1002 may control the thermal control module 1010 to deliver power according to a thermal profile. In the mixed mode, the processing module 1002 may control the thermal control module 1010 to deliver power according to a thermal profile and/or user input.

The thermal device 1000 (e.g., memory 1020) may store thermal profiles that include data indicating how to deliver power to one or more thermal units 1016. For example, the thermal profiles may include data indicating the voltage (e.g., analog voltage level and/or digital average with PWM) to apply to one or more thermal units 1016 over time. As another example, the thermal profiles may include data indicating the current to deliver to one or more thermal units 1016 over time. A thermal profile may include one or more thermal unit profiles. A thermal unit profile may include data indicating how to deliver power to a single thermal unit 1016 (e.g., between two electrical contacts connected to the thermal unit). In one example, if the thermal device 1000 includes two thermal units, the thermal profile may include two thermal unit profiles.

The thermal profile (e.g., including multiple thermal unit profiles) can be stored in a variety of ways. In general, the data stored in the thermal profile indicates to the processing module 1002 and thermal control module 1010 how to deliver power to the thermal unit(s) 1016. In some implementations, the thermal profile may include a plurality of digital values indicating current/voltage to be delivered to the thermal unit(s) 1016 over time. In other examples, the thermal profile may be stored as a function that yields current/voltage over time. Note that in some cases, the values stored in the thermal profiles may not be voltage/current values over time, but instead may be digital values (e.g., PWM control values) used by the processing module 1002 and/or thermal control module 1010 to cause power to be delivered to the thermal unit(s) 1016 over time.

FIGS. 11A-11B illustrate example operating curves for thermal units 200, 1016 (e.g., current versus time curves). The thermal device 100, 1000 may control the thermal units 200, 1016 (e.g., applied voltage/current) according to a thermal profile stored on the thermal device 100, 1000. FIG. 11A illustrates an operating curve (e.g., current versus time) for a single thermal unit. FIG. 11B illustrates operating curves (e.g., current versus time) for two thermal units.

In FIG. 11A, the device electronics control the thermal unit according to the thermal unit profile to operate in the off state, heating state, and the cooling state. The device electronics may transition the thermal unit between the three states. For example, in FIG. 11A, the device electronics initially set the thermal unit into the off state and then transition the thermal unit to the heating state. Next, the device electronics transition to the thermal unit to the off state and then the cooling state. In some cases, the device electronics may transition the thermal unit between states in a stepwise fashion. In other cases, the device electronics may transition the thermal unit between states in a more gradual fashion, illustrated as a sloped line in FIG. 11A.

FIG. 11B illustrates two separate thermal unit profiles. In FIG. 11B, the device electronics may independently control two thermal units, each according to separate thermal unit profiles. The separate thermal unit profiles may be part of a thermal profile executed by the thermal device. Note that the independently controlled thermal units may be operated in different states at the same time. Since the thermal units may be associated with different portions of the user's body, controlling the thermal units according to the thermal unit profiles of FIG. 11B may result in one portion of the user's body being cooled while another portion of the user's body is heated.

FIGS. 11A-11B illustrate a variety of different heating/cooling patterns. Thermal profiles may include patterns similar to, or different from, the illustrated patterns (e.g., regular/irregular/repetitive/non-repetitive). Additionally, a thermal profile may include patterns that transition from repetitive to non-repetitive and/or from regular to irregular (or vice versa) over time. As described herein, a user may create new thermal patterns or modify existing thermal patterns while using the thermal device or working offline.

The duration of heating/cooling pulses (e.g., square pulses) deliverable by the thermal device may vary depending on a variety of parameters. In some implementations, the duration of the pulses may be selected based on response times of the thermal device and/or the user's ability to perceive the heating/cooling. For example, response times of the thermal device (e.g., thermal units) affecting the time required to provide heating/cooling to a user may determine the minimum duration of the pulses. As another example, a user's ability to perceive the changes in heating/cooling being delivered may determine the minimum duration of the pulses. For example, if a user is unable to differentiate pulses having a duration of less than one second from pulses having a duration of one second, then the minimum pulse duration may be set to one second. The ability of a user to perceive changes in heating/cooling may depend on the region of the body to which the thermal device is applied. Accordingly, the minimum duration of pulses may also depend on where the thermal device is to be applied. In some implementations, the pulses may have a duration on the order of a second or more, although the pulses may be set to a duration of less than a second if perceptible by the user.

In some implementations, the thermal device can control power delivered to the thermal units based on a sensed and/or estimated temperature. For example, the thermal device may control the delivery of power to meet a target temperature that is adjustable by the user. As another example, the thermal device may control the delivery of power such that the temperature remains greater than a threshold temperature, such as a temperature threshold set by a user or a minimum allowable temperature (e.g., in factory settings).

The thermal device can control heating/cooling based on the temperature of the thermal device in proximity to the user (e.g., the temperature of a thermal zone). In some implementations, the thermal device can include one or more temperature sensors that sense temperatures in one or more thermal zones. In implementations where the thermal device includes one or more temperature sensors, the thermal device can control heating/cooling based on temperature indicated by the temperature sensor.

In implementations where the thermal device 1000 does not include a temperature sensor, the processing module 1002 may estimate the temperature and control heating/cooling based on the estimated temperature. The processing module 1002 may estimate the temperature based on one or more factors, such as the amount of power delivered to the thermal units 1016 (e.g., voltage/current) and the amount of time over which the power has been delivered. In some implementations, the memory 1020 may include temperature estimation models and/or tables that the processing module 1002 may use to estimate temperature. For example, the models/tables may indicate an estimated temperature for power values and/or a thermal profile over time. The processing module 1002 may also determine the temperature based on a combination of temperature indicated by the temperature sensors and the estimated temperature. In some implementations, the memory 1020 may include models/tables that use sensed temperatures to estimate additional temperatures.

Although the thermal device 1000 can control heating/cooling based on temperature (e.g., a target temperature), in some implementations, the thermal device 1000 can control heating/cooling based on alternative and/or additional parameters, such as an amount of energy/heat withdrawn from a user and/or delivered to a user. For example, the thermal device 1000 may control heating/cooling to reach a target amount or rate of energy delivered/withdrawn. The thermal device 1000 may determine the amount of energy delivered/withdrawn based on a variety of parameters, such as the delivered current/voltage to the thermal units 1016 and the amount of time over which the current/voltage was delivered.

In some implementations, the thermal device 100 may include components that indicate an amount of pressure placed on the thermal device 100 (e.g., a pressure sensor). Such components may be embedded in and/or attached to the package substrate or device packaging. In these implementations, the thermal device 100 may control heating/cooling based on the indicated pressure (e.g., as indicated by the pressure sensor). In one example, the thermal device 100 may reduce an amount of heating/cooling if the pressure sensing components indicate that the thermal device 100 is pressed more firmly against the user, as the pressure may be indicative of a close contact and better heat transfer between the thermal device 100 and the user. In another example, the thermal device 100 may be configured to increase heating/cooling in response to increased pressure placed on the thermal device 100. In this example, if a user presses their hand on top of the thermal device 100 to increase pressure on the thermal device 100, the thermal device 100 may respond by delivering more heating/cooling to the area.

FIGS. 12A-12C illustrate example methods describing operation of the thermal device 100 in different modes of operation. FIG. 12A illustrates an example method describing operation of the thermal device 100 in the manual mode. In FIG. 12A, the thermal device 100 is initially started in block 1200 (e.g., using an on/off button, or based on motion sensing inputs). In block 1202, the thermal device 100 (e.g., the device electronics) sets an initial power delivery to the one or more thermal units 200. In block 1204, the thermal device 100 waits for user input, which may include user interaction with manual controls (e.g., user input buttons) on the thermal device 100 and/or user interaction with a GUI on the user device 104. Example user input may include incrementing/decrementing heating/cooling (e.g., power delivery). If the thermal device receives user input in block 1204, the thermal device 100 may modify power delivery to the one or more thermal units 200 according to the user input in block 1206.

FIG. 12B illustrates an example method describing operation of the thermal device 100 in the automatic mode. In FIG. 12B, the thermal device 100 is initially started in block 1210. Upon starting, the thermal device 100 may load a thermal profile in block 1212. For example, the thermal device 100 may load a stored thermal profile or may receive a thermal profile from the user device 104. In block 1214, the thermal device 100 controls heating/cooling (e.g., power delivery) according to the loaded thermal profile.

FIG. 12C illustrates an example method describing operation of the thermal device 100 in the mixed mode. In blocks 1220-1224, the thermal device 100 is initially started, loads a thermal profile, and controls heating/cooling according to the thermal profile, as described with respect to FIG. 12B. In the mixed mode, the user may modify the thermal profile and/or load another thermal profile onto the thermal device 100 in block 1226. For example, the user may provide user input that modifies the currently running thermal profile via manual controls on the thermal device 100 and/or GUI controls on the user device 104. The user may also load new thermal profiles to run on the thermal device 100. For example, the user may select a new thermal profile stored on the thermal device 100 or download a thermal profile from the user device 104 to the thermal device 100. In block 1228, the thermal device 100 may run the new profile until the user modifies the new profile and/or loads another thermal profile.

FIG. 13 illustrates a plurality of user devices 1300-1, 1300-2, . . . , 1300-N in communication with a remote server 1302 via a network 1304. Each of the user devices 1300 is in communication with a different thermal device 1306-1, 1306-2, . . . , 1306-N. In FIG. 13, different users may each own/operate one of the user devices 1300 and one of the thermal devices 1306. The remote server 1302 may be owned/operated by a party other than the users. For example, the remote server 1302 may be operated by the developer/manufacturer of the thermal devices 1306. In these examples, the developer/manufacturer of the thermal devices 1306 can provide data and programs to the remote server 1302 for download by the user devices 1300.

In some implementations, the remote server 1302 can provide one or more programs (e.g., applications) to the user devices 1300. The one or more programs may be executed by the user devices 1300 to interact with the thermal devices 1306. For example, the one or more programs may generate GUIs on the user device 1300 which the user may use to interact with the thermal device 1306 (e.g., see FIGS. 17A-17K). The user devices 1300 may download and execute the one or more programs in order to interact with the thermal devices 1306 (e.g., after the users purchase the thermal devices).

In some implementations, the remote server 1302 may store data that can be accessed by the user devices 1300. For example, the remote server 1302 can store thermal profiles. In some implementations, the thermal profiles may be created by the owner/operator of the remote server 1302 and uploaded to the remote server 1302. In another example, the thermal profiles may be created by one or more of the users and uploaded to the remote server 1302. Users may download the thermal profiles and load the thermal profiles on their thermal devices 1306. Providing the thermal profiles for download may help new and existing users conveniently acquire and try new thermal profiles.

A thermal profile may also include associated data. The associated data may include thermal device information that indicates the type of thermal device with which the thermal profile may be used. In one example, the associated data may include thermal device identification numbers (e.g., model numbers) indicating the type of thermal device with which the thermal profile is compatible. As another example, the associated data may indicate that the thermal profile should be used with a certain device having a certain configuration of thermal units and/or sensors.

In some implementations, the users can store user data on the remote server 1302. Example user data may include the types of conditions for which the user uses the thermal device 1306 along with data indicating how effective various thermal profiles are in alleviating the condition. For example, the user may upload a thermal profile and additional data along with the thermal profile indicating the condition for which the thermal profile is used and how effective the thermal profile is in alleviating the condition (e.g., a score from 1-10). The remote server 1302 can make recommendations to users based on uploaded user data. For example, the remote server 1302 can recommend thermal profiles to users with a condition if the thermal profiles are indicated as effective by other users for the same/similar conditions. The remote server 1302 can also recommend additional activities or behaviors that can help the user while using the thermal device 1306 (or independent of thermal device usage). These recommendations can include exercise guidance or stretching suggestions to reduce pain in a given part of the body. In some implementations, the user device 1300 may also make recommendations without communication with the remote server 1302. For example, the user device 1300 may make recommendations based on detected motion over time. The GUI can provide notifications/recommendations to the user (e.g., that the user stretch, increase activity, etc.). In a specific example, with respect to low back pain, the user device 1300 can alert the user that they should move around and do some stretching/exercises to help reduce the pain that they are feeling. In this way, the remote server 1302, user device 1300, and thermal device 1306 are together able to provide a well-rounded therapeutic solution for the user.

FIGS. 14A-14B illustrate heat flow between thermal units 1400-1, 1400-2 and the user's body. The thermal units 1400 include thermal reservoirs 1402-1, 1402-2. In FIG. 14A, the thermal units 1400 each include their own independent thermal reservoirs 1402. In FIG. 14B, two adjacent thermal units 1400 share the same thermal reservoir 1404. In FIG. 14B, the thermal unit 1400-1 on the left is operating in the cooling state and the thermal unit 1400-2 on the right is operating in the heating state. If the thermal reservoir 1404 between the two thermal units 1400 has a sufficient thermal conductivity, the thermal reservoir may transfer heat from one thermal unit to the other. In this manner, the thermal reservoir 1404 may complete a “thermal circuit” that includes the user's body, the thermal units 1400, and the thermal reservoir 1404. For example, heat may be withdrawn from the user's body by the thermal unit 1400-1 in the cooling state, transferred through the thermal reservoir 1404, and then put back into the user's body by the thermal unit 1400-2 operating in the heating state. Additionally, the thermal reservoir 1404 may have a sufficient heat capacity to sink/source heat if there is any heat transfer imbalance between the thermal units 1400.

FIGS. 14C-14D illustrate two thermal units 1400 that share a thermal bridge 1406. FIG. 14E is a perspective view of the thermal bridge 1406 across two thermal units 1400. A thermal bridge described herein may refer to a thermally conductive material that is configured to transfer heat between thermal units and/or the user's body. For example, with respect to FIG. 14C, the thermal bridge 1406 transfers heat between two thermal units 1400. As another example, with respect to FIG. 15A, the thermal bridge 1500 transfers heat between one side of a thermal unit and the users body adjacent to the thermal unit. As another example, with respect to FIG. 15L, the thermal bridge 1500-12 transfers heat between multiple thermal units and multiple different portions of the user's body.

In general, a thermal bridge may be formed from a thermally conductive material, such as a metal (e.g., solid and/or woven metal mesh), thermally conductive polymer, thermally conductive carbon structure, a semiconductor material, and/or thermally conductive liquid. In some implementations, a thermal bridge may be thermally conductive such that heat transferred into the thermal bridge is transferred across the thermal bridge and back into another thermal unit and/or the user's body. As described herein, a thermal reservoir may also transfer heat from one thermal unit to another thermal unit and/or the user's body. Accordingly, the thermal reservoir and the thermal bridge described herein may have similar functionality. However, the thermal bridges that are illustrated and described herein may generally have a higher conductivity and lower heat capacity than the thermal reservoirs. In implementations of the thermal device where both thermal conductivity and heat capacity are desirable, the thermal devices may include both thermal bridges and thermal reservoir material (e.g., FIGS. 15I-15L). In some implementations, the thermal bridge may be formed from a flexible material, such as the flexible thermal bridge 1408 illustrated in FIG. 14F.

Heat flow between adjacent thermal units 1400 is illustrated in FIGS. 14C-14D. In FIG. 14C, one thermal unit 1400-1 is operating in the heating state while the other thermal unit 1400-2 is operating in the cooling state. The thermal bridge 1406 transfers heat from the thermal unit 1400-2 operating in the cooling state to the thermal unit 1400-1 operating in the heating state. In FIG. 14D, the states of the thermal units 1400 are reversed, as is the direction of heat transfer through the thermal bridge 1406.

FIGS. 14G-14J illustrate operation of thermal units in different states (e.g., in the manner illustrated in FIGS. 14C-14D). In FIG. 14G, two thermal units 1410-1, 1410-2 are connected to one another via a thermal bridge 1412. One thermal unit 1410-2 operates in a heating state while the other thermal unit 1410-1 operates in a cooling state. In FIG. 14G, heat may flow in a “thermal circuit” created by the thermal units 1410, thermal bridge 1412, and the user's body. The thermal device may operate in a thermal equilibrium in cases where the heat absorbed by one thermal unit is approximately equal to the heat transferred by the other thermal unit. The thermal units may operate in a thermal equilibrium for a period of time without excess heating in the thermal bridge that may alter operation of the thermal units. Note that heat may be generated (e.g., resistive heat losses) during operation of the thermal units. Heat generated during operation may tend to cause the thermal units to fall out of thermal equilibrium over time if it is not adequately transferred to the user's body (or other heat sink/reservoir).

The user's perception of hot and cold may also play a role in operating the thermal units to achieve an equilibrium. In some cases, a given power density (e.g., 1 W/cm\hat{ }2) may have a greater perceived effect when heat is flowing into the body (heating) as opposed to flowing out of the body (cooling). Accordingly, the same 1 W/cm\hat{ }2 heat load leaving the body may be less perceptible. Therefore, the ability to maintain thermal equilibrium without a thermal reservoir may be governed by the comfort threshold on the heating side of the system. As a result, for a given amount of heating, heating may be spread out across a larger surface area to prevent reaching an uncomfortable heating power density (W/cm\hat{ }2).

FIGS. 14H-14I illustrate how multiple thermal units 1414 connected to a single thermal bridge 1414 may be operated to achieve a thermal equilibrium. In FIGS. 14H-14I, different numbers of thermal units may be operated at selected intensity levels over time to achieve a thermal equilibrium. For example, in order to increase the amount of heat delivered to the body, the device electronics may operate more of the thermal units in the heating state and/or increase the amount of heating in each of the thermal units currently operating in the heating state. As illustrated in FIG. 14H, the device electronics may also set a thermal unit into the OFF state while operating other thermal units in the heating/cooling states. FIG. 14I illustrates a variety of different ratios of heating/cooling thermal units in a variety of different patterns.

FIG. 14J is an example pair of waveforms that illustrate how the device electronics can control thermal equilibrium over a period time. In FIG. 14J, the broken line illustrates heating intensity (e.g., from one or more thermal units), and the solid line illustrates cooling intensity (e.g., from one or more thermal units). The heating and cooling may be provided in different thermal zones under the same thermal bridge. Initially (e.g., during period A), the device electronics maintain a low level of heating along with short pulses of cooling that balance out the low level heating. The device electronics then increase the amount of heating delivered to the user during period B. In order to balance out the increased level of heating, the device electronics increase the pulse duration of cooling delivered to the user.

FIGS. 14K-14M illustrate example combinations of thermal bridges and thermal reservoir material. In FIG. 14K, the thermal reservoir material 1420 is deposited over the thermal bridge 1422. In FIG. 14L, the thermal reservoir material is included in a cavity 1424 defined by the thermal bridge 1426. FIG. 14M is a perspective view of the thermal bridge 1426 illustrated in FIG. 14L, including caps 1428 that may seal the cavity 1424. The thermal bridge 1426 of FIGS. 14L-14M may be filled with a thermal reservoir material, such as a phase-change material. The thermal reservoir material may absorb/release heat over time, depending on how the thermal units associated with the thermal bridge 1426 are being operated. Addition of the thermal reservoir material onto/within the thermal bridge may extend the period of time for which the thermal units can operate at their desired rates of heat transfer.

FIGS. 15A-15L illustrate example thermal bridges 1500-1, 1500-2, . . . , 1500-12 that interface with one or more thermal units and the user's body. In FIG. 15A, the thermal unit 1502 interfaces with the user's body on one side. The opposite side of the thermal unit 1502 interfaces with the thermal bridge 1500-1. The thermal bridge 1500-1 extends from the opposite side of the thermal unit 1502 along the depth of the thermal unit to interface with the body. The thermal bridge 1500-1 may define a region that conforms to the shape of the thermal unit 1502. The thermal unit 1502 is located within the region defined in the thermal bridge 1500-1. In some implementations (e.g., FIGS. 15E-15G), the thermal bridge may encircle the thermal unit and contact the user's body around the circumference of the thermal unit.

The thermal bridge 1500-1 may complete a thermal circuit between the body and the thermal unit 1502. For example, if the thermal unit 1502 is operating in the cooling state, the thermal bridge 1500-1 may transfer heat to the body that is then withdrawn into the thermal unit 1502. As another example, if the thermal unit 1502 is operating in the heating state, the thermal bridge 1500-1 may withdraw heat from the body that is then transferred back into the body by the thermal unit 1502.

As illustrated in FIGS. 15A-15L, the thermal bridge may be configured in a variety of different ways. In some implementations, the thermal bridge may be insulated from the thermal unit. For example, in FIG. 15B, the thermal bridge 1500-2 is insulated by insulation 1503 from the thermal unit 1504 and a thermally conductive body contact layer 1506. In some implementations, the thermal bridge may include a thermal reservoir material deposited on the thermal bridge. For example, the thermal bridges 1500-3, 1500-4 of FIGS. 15C-15D include a thermal reservoir material 1508. In FIG. 15D, the thermal bridge 1500-4 defines reservoirs into which the thermal reservoir material 1510 may be deposited. The deposited thermal reservoir 1510 is then covered with a cover material 1512. In some implementations, the cover material 1512 may be formed from a film or foil designed to capture the thermal reservoir material 1510 inside its pocket. FIGS. 15E-15G illustrate perspective views of additional thermal bridge geometries, each of which includes a reservoir 1514-1, 1514-2, 1514-3 into which thermal reservoir material may be deposited. The thermal bridges 1500-5, 1500-6, 1500-7 each include a contoured portion 1516-1, 1516-2, 1516-3 under which one or more thermal units may be placed. The example contours of FIGS. 15E-15G are rectangular, circular, and hexagonal. The cross sections of the thermal bridges of FIGS. 15E-15G may be similar to the thermal bridge illustrated in FIG. 15D.

Thermal bridges may be formed from flexible and/or rigid material. The thermal bridge 1500-8 illustrated in FIG. 15H is flexible. Example flexible thermal bridge materials may include, but are not limited to, metal mesh/woven materials (e.g., copper braided material or similar), thin metallic materials (e.g., copper or aluminum sheet), and thermally conductive polymers.

The thermal bridges 1500-9, 1500-10, 1500-11, 1500-12 of FIGS. 15I-15L define one or more cavities 1518-1, 1518-2, 1518-3, 1518-4, 1518-5 that may be filled with thermal reservoir material. The thermal bridge 1500-9 of FIG. 15I defines two separate cavities 1518-1 that may be filled with thermal reservoir material. The thermal bridge 1500-10 of FIG. 15J defines a single cavity 1500-2. As illustrated in FIG. 15I and other figures, a variety of different cavity geometries may be used to promote thermal conduction between the thermal bridges and the thermal reservoir material. In some implementations, such as FIG. 15K, insulation material 1522 may insulate the thermal bridge 1500-11 from the thermal unit and/or a thermally conductive body contact layer 1520. As described herein, a thermal bridge may interface with one or more thermal units and/or one or more portions of the user's body. FIG. 15L illustrates an example thermal bridge 1500-12 that interfaces with two thermal units and three portions of the user's body.

In some implementations of the thermal device 100, the thermal device 100 may include one or more heating elements that may be used to deliver heat to the user's body. In some implementations, the thermal device 100 may use heating elements and thermal units to provide heat to the user. In other implementations, the thermal device 100 may use thermal units to cool the user and heating elements to heat the user. The device electronics (e.g., the thermal control module 1010) can deliver voltage/current to the heating elements (e.g., according to the thermal profile) to control the heat delivered to the body via the heating elements.

FIG. 16A illustrates an example heating unit 1600 that may be included in the thermal device 100. A heating unit 1600 can include a heating element 1602 and a substrate 1604. The heating element 1602 can generate heat that is applied to a user's body (e.g., via resistive heating). For example, the heating element 1602 may include a metallic wire that generates heat when power is delivered to the heating element 1602. The heating element 1602 may include electrical contacts 1606 for connection to the device electronics. The substrate 1604 can provide support to the heating element 1602 (e.g., to maintain shape) so that the heating element 1602 can be positioned near the user's body. For example, the heating element 1602 can be attached to the substrate 1604 and/or formed on the substrate 1604 (e.g., etched on the substrate). The substrate 1604 can be composed of a flexible material and/or a rigid material (e.g., polyester, polyimide, and/or silicone).

FIGS. 16B-16H illustrate different configurations of thermal units and heating units. In some implementations (e.g., FIG. 16B), a heating unit 1608 can be placed between thermal units 1610-1, 1610-2 in the thermal device. In other implementations (e.g., FIG. 16C), a heating unit 1612 can be placed under the thermal unit 1614 such that the heating unit 1612 interfaces with the user's body. In these implementations, the thermal unit 1614 may heat/cool the user through the heating unit 1612. FIG. 16D illustrates an example combination heating/thermal unit. In FIG. 16D, the thermal elements 1616 may be added to the substrate 1618 in the same manner described with respect to FIGS. 5A-5D. In FIG. 16D, the heating element 1620 is fabricated onto/within the substrate 1618 under the thermal elements 1616.

When the heating elements are arranged under the thermal units, the device electronics may control the heating elements and thermal units such that the thermal zone under the units is either being heated by the heating units or cooled by the thermal units. For example, to cool the thermal zone, the device electronics may set the thermal unit in the cooling state and turn off power to the heating element. To subsequently heat the thermal zone, the device electronics may set the thermal unit into the off state and deliver power to the heating element. In some implementations, the device electronics may set the thermal unit into the heating state to heat the underlying thermal zone as well. FIG. 161 illustrates an example heating element profile and thermal unit profile for a single thermal zone. In FIG. 161, the device electronics control heating in the thermal zone by setting the thermal unit into the off state while delivering power to the heating element. Also, in FIG. 161, the device electronics control cooling in the thermal zone by setting the thermal unit into the cooling state while turning off power to the heating element. The heating element profile and the thermal unit profile of FIG. 161 may be included in an overall thermal profile for the thermal device.

FIGS. 16E-16F illustrate a portion of an example thermal device in which a heating unit 1622 is included between two thermal units 1624-1, 1624-2 (including thermal reservoirs 1628). Insulation 1626 is included over top of the heating unit 1622 to insulate the thermal units 1624 from the generated heat and to direct heat to the user's body. As illustrated in FIG. 16F, the thermal units 1624 and heating unit 1622 may be flexible (e.g., include flexible substrates). When the thermal units 1624 and heating unit 1622 are arranged side by side, the device electronics may control heat in the thermal zones under the heating unit by delivering power to the heating elements. For the thermal zones under the thermal units 1624, the device electronics may control heating/cooling as described herein. The device electronics may control the heating unit 1622 and thermal units 1624 to heat at the same time or at different times. For example, with respect to FIG. 16J, the heating element profile may represent the delivery of voltage/current to the heating element to heat thermal zones underlying the heating element. The thermal unit profile may represent the delivery of voltage/current to the thermal unit to cool the thermal zones underlying the thermal units. The heating element profile and the thermal unit profile of FIG. 16J may be included in an overall thermal profile for the thermal device.

FIGS. 16G-16H illustrate fabrication of a thermal device in which a plurality of thermal units 1630 is placed over top of a corresponding plurality of heating units 1632. The thermal device of FIGS. 16G-16H also include a thermal reservoir 1634 over top of the thermal units 1630. The thermal reservoir 1634 can be used to absorb or deliver heat to the thermal units 1630 during use. The thermal reservoir 1634 may be removable from the thermal device. In some implementations, the thermal reservoir 1634 may be cooled to a temperature below body temperature. For example, the thermal reservoir 1634 may include ice or another cooling material. In other implementations, the thermal reservoir 1634 may be heated (e.g., above body temperature). In some cases, the thermal device can provide cooling to the user even though the thermal device is operating in a heating state (between the thermal reservoir and the user's body). The same effect can be reversed if the thermal reservoir 1634 is pre-heated (e.g., the thermal device can provide heat to the user) even if it is operating in a cooling state locally.

FIGS. 17A-17K illustrate example GUIs that can be displayed on the user device 104. Users may use the example GUIs to: 1) control the thermal device, 2) transfer data to the thermal device, 3) retrieve data from the thermal device, 4) transfer data to the remote server, 5) retrieve data from the remote server, and perform other operations, such as creating and modifying thermal profiles. In FIGS. 17A-17K, the user devices 1700-1, 1700-2, . . . , 1700-11 include a touchscreen that overlays the GUIs. A user can interact with the GUI by interacting with the touchscreen display (e.g., touching/swiping the touchscreen display). In other implementations, a user device 104 may include additional user inputs, such as buttons, that the user may use to control the thermal device. The GUIs of FIGS. 17A-17K are only example GUIs used to illustrate various example features of the user device, and as such, do not represent an exhaustive set of features that may be provided by the user device.

FIG. 17A illustrates a GUI that the user may use to control the thermal device (e.g., in the manual mode). In FIG. 17A, the GUI controls a thermal device having two thermal zones, where each thermal zone includes one or more thermal units. The user can interact with two different GUI elements (e.g., sliders 1702), each of which controls heating/cooling within different thermal zones. For example, the user may slide (e.g., swipe) the slider icons 1702 in the Hot/Cold direction to control the amount of heating/cooling in the thermal zones. Although sliding GUI elements are illustrated, in other implementations, other GUI elements may be used to control heating/cooling, such as graphical buttons (e.g., +/− buttons) or dials.

FIGS. 17B-17C illustrate GUIs that provide information to the user, provide controls for the user, and acquire feedback from the user. The GUI in FIG. 17B indicates that the user device is connected to the thermal device. The GUI also gives the user various controls for the thermal device. For example, the user can: 1) update the active thermal profile running on the thermal device, 2) view the active thermal profile in real-time in another GUI, and 3) put the thermal device to sleep. Additionally, the GUI prompts the user for feedback indicating how effective the thermal profile is for the user.

FIG. 17C illustrates a GUI that allows the user to select a new thermal profile to run on the thermal device and/or modify a current thermal profile. The user can select a new thermal profile from other users (e.g., from the remote server), select a profile saved on the user device or remote server, or select a random profile. The user can also create a new profile. In some implementations, the thermal profiles can be assigned names (e.g., by the user/creator) so that the user can identify the thermal profile.

Additionally, the user may use motion sensors or music to generate a profile. In the case of generating profiles based on motion, the thermal device may detect motion patterns from the motion sensor (such as a walking motion) and/or may respond to real-time changes in the user's motion. For example, the thermal device may detect a regular periodic frequency within the user's motion. In response to this detected frequency, the thermal device can deliver pulses of heating/cooling to coincide with the user's motion. Further, in order to have the pulse of heating/cooling arrive at the user's body in-phase with his/her periodic motion, the thermal device may delay/offset the pulse of heating/cooling by a given amount (based on the thermodynamic properties of the device package). In the case of generating profiles based on music, the user may choose an audio stream on the user device (either downloaded onto the user device or streaming on the internet). The audio stream's contents can be processed (e.g., by an external computing device and/or the thermal device) to find underlying rhythms and frequency patterns, which can then be converted to heating/cooling delivery profiles. For example, if an audio stream has a melody that rises and falls at a given rate, then a profile can be created to match it. Introducing a time offset in the music stream can allow for the timing of the music to match the heating/cooling felt by the user. This time offset can account for the time needed for the thermal gradients to be created by the thermal device. A benefit of using music as a seed for generating new profiles is that it allows for varied and diverse profiles without the need for a high degree of user input. Another example benefit of using music to generate profiles is that the user may listen to the music while experiencing the music-generated profile, so that the effect of the thermal device is combined with the effect of hearing the music stream.

FIG. 17D illustrates a GUI that allows a user to create a custom thermal profile. In the GUI, the user may draw a pattern (e.g., with their finger or stylus). The user may then save the pattern (i.e., thermal profile) and upload the pattern to the thermal device. The user can retrieve and modify the saved pattern at a later time.

FIGS. 17E-17F illustrate GUIs that allow a user to specify their desires for a thermal profile, which may then be generated automatically by the user device. In FIG. 17E, the user can adjust a slider 1704 left or right to indicate that they would like maximum thermal intensity or maximum thermal device operating time. In general, a greater amount of heating/cooling may yield a shorter operating time when the thermal device is running on a battery. The GUI provides the user with the choice of whether to increase thermal intensity or increase operating time. The thermal device may adjust the amplitude of the current pattern according to the user's selection and/or select another thermal pattern based on the selected operating time and/or thermal intensity.

The GUI of FIG. 17F illustrates a graph with four quadrants and a point 1706 that the user may position within the quadrants to control the thermal intensity and whether the heating/cooling is steady or in pulses. The user may drag the point 1706 in the X direction to increase/decrease the thermal intensity. The user may drag the point 1706 in the Y direction to modify the rate of heating/cooling pulses delivered to the user. For example, dragging the point 1706 toward the pulses portion of the Y axis may cause an increase in pulse frequency, whereas dragging the point 1706 toward the steady portion of the Y axis may cause the pulse frequency to decrease (e.g., steady=no pulses).

FIG. 17G illustrates a GUI that conveys thermal device information to the user, including: 1) the connection status between the user device and the thermal device, 2) the battery status of the thermal device, and 3) the remaining operating time for the thermal device at the current settings (e.g., the current thermal profile). The GUI also illustrates a thermal map of the thermal device that indicates heating/cooling in different thermal zones. Additionally, the GUI illustrates the thermal profile running in zone 1 of the thermal device. Over time, the illustrated thermal profile may scroll from left to right as the thermal device executes the thermal profile. This allows the user to visualize the past/present/future behavior of the thermal profile. The user may pause the thermal device by pressing the “PAUSE DEVICE” button in the GUI.

FIG. 17H illustrates a GUI that allows the user to select a desired usage (operation) time for the thermal device. For example, the user may slide the slider 1708 to the right/left to increase/decrease the usage time. The user device and/or the thermal device may then update the current thermal profile or generate a new thermal profile based on the selected usage time.

FIG. 17I illustrates a GUI that allows the user to control how long a thermal profile is run and how long a thermal profile is turned off. For example, the user may use a slider GUI element to set an on time that sets how long the thermal profile should run. The user may also use a slider GUI element to set an off time that sets how long the thermal device should cease heating/cooling (e.g., pause) after running for the on time. The thermal device may then repeat the on/off behavior for the selected on/off times. The user can also edit the settings for different thermal zones using the GUI. The user device may calculate the estimated usage time for the thermal device according to the present battery level, the on/off times, and the thermal intensity. The GUI displays the estimated usage time to the user (e.g., 3 hours, 10 min). Modifying the on time and off time can extend/reduce the battery life (i.e., the estimated usage time) of the thermal device.

FIG. 17J illustrates a GUI that allows a user to tailor the motion response of the thermal device. As described herein, the thermal device can determine the motion of the user based on a motion sensor included in the thermal device and/or a motion sensor included on the user device. The user may move the slider GUI element to the left or right to adjust whether the thermal device provides more heating/cooling while the user is stationary or moving or in a given orientation/position.

FIG. 17K illustrates a GUI that acquires user information. The GUI prompts the user to describe their pain based on whether the user is stationary/moving. The GUI also prompts the user to describe their pain in terms of whether it is consistent/steady or shooting. Additionally, the GUI prompts the user to indicate their source of pain. The user information acquired via the GUI may be stored on the user device and/or the remote server. At a later time, the user may indicate which thermal profile(s) are most effective in comforting the pain described in the GUI. The effectiveness of one or more thermal profiles with respect to the reduction/elimination of pain described in the GUI may be stored at the remote server and/or user device and be used to make recommendations to the user or other users, as described herein.

The thermal device 100 can include a device package that can house one or more thermal units, heating units, thermal reservoirs, thermal bridges, device electronics, and other components (e.g., a battery). The device package may include flexible portions that conform to a user's body. FIGS. 1A-1C and 18A-24B illustrate different example thermal devices having different packages.

FIGS. 1A-1C illustrate a first thermal device 100-1, as described above. FIGS. 18A-18D illustrate a second thermal device 100-2. The second thermal device package can include one or more thermal/heating units arranged in any manner throughout the package. The second thermal device 100-2 can be applied to different parts of the user's body. The second thermal device package and the first thermal device package can include one or more belt loops 1800 that receive a belt 1802 used to hold the thermal device to a user's body (see FIG. 1C). The belt 1802 can include a belt clasp 1803 for fastening ends of the belt 1802 together. With respect to FIG. 18A, the second thermal device 100-2 can include a user input button 102 (e.g., an on/off button) and a power input port 108.

FIG. 18B illustrates an exploded view of the second thermal device 100-2. The second thermal device 100-2 includes an encapsulation. The encapsulation is formed from an encapsulation top cover 1804-1 and an encapsulation bottom cover 1804-2. The encapsulation encapsulates components of the thermal device 100-2, such as the thermal/heating units, package substrate 300, battery 304, and device electronics. The top/bottom covers 1804-1, 1804-2 in FIG. 18B can be flexible material that can be adhered together or connected in another manner, such as fused, vulcanized, ultrasonically welded, or thermally welded. In some implementations, the encapsulation may not entirely cover the package substrate including the thermal/heating units. In these implementations, the package substrate 300, or other body contact layer (e.g., a thermally conductive layer) may contact the user (e.g., body or clothing). The encapsulation 1804 may be formed from materials including, but not limited to, cloth-based or fabric materials, molded flexible plastics/rubbers, foams, and synthetic fleece material. In some implementations, the thermal device 100-2 may include material/structure that imparts some rigidity to the thermal device. FIG. 18D illustrates an additional adhesive layer 1806 that may be attached to the encapsulation bottom cover 1804-2.

The second thermal device 100-2 of FIG. 18B includes a package substrate 300 that may include thermal/heating units (not illustrated). Additionally, the second thermal device 100-2 may include a thermal reservoir/bridge layer 1810. In some implementations, the thermal reservoir 1810 layer may be made thicker than that illustrated in FIG. 18B in order to provide more thermal mass.

The thermal devices of FIGS. 18A-23A are illustrated as thinner than the thermal device 100-1 of FIGS. 1A-1C. This is because the thermal device 100-1 of FIGS. 1A-1C may include one or more thicker thermal reservoirs, whereas the thermal devices of FIGS. 18A-23A may include thinner thermal reservoir layers, or no thermal reservoir layer at all. Each of the thermal devices of FIGS. 18A-23A may be modified to include additional thermal reservoir material. In implementations of the thermal devices of FIGS. 18A-23A including additional thermal reservoir material, the devices of FIGS. 18A-23A may be made thicker. The increase in thickness of the thermal devices may correspond to the thickness of added thermal reservoir material.

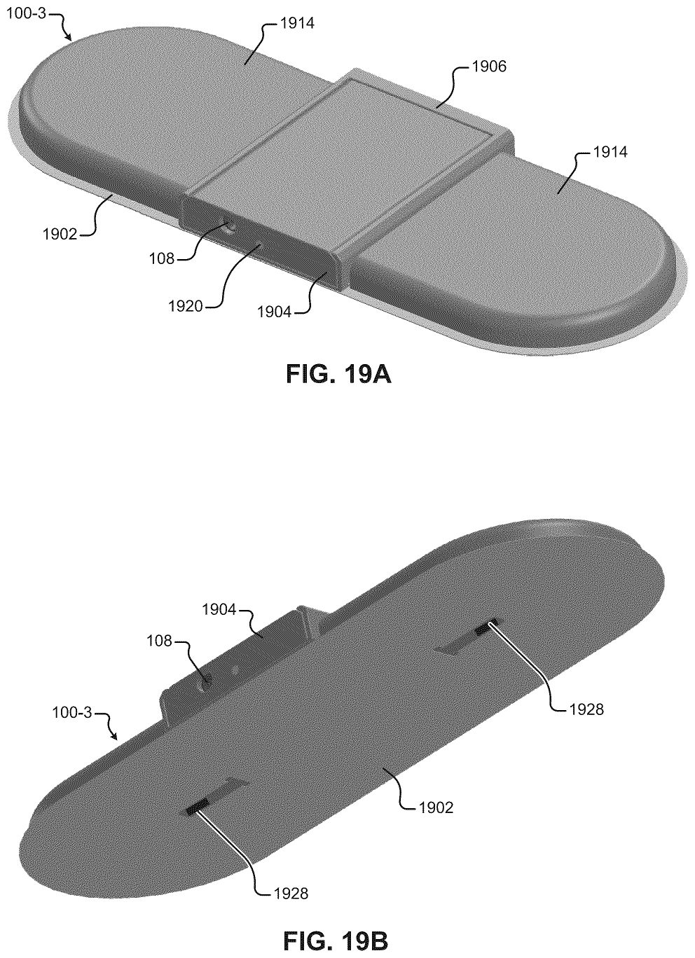

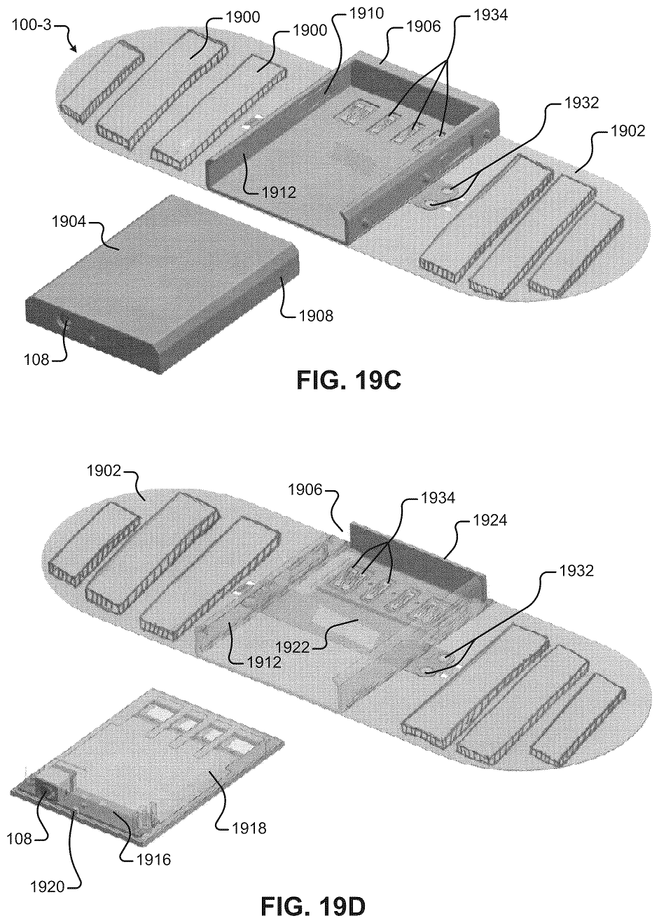

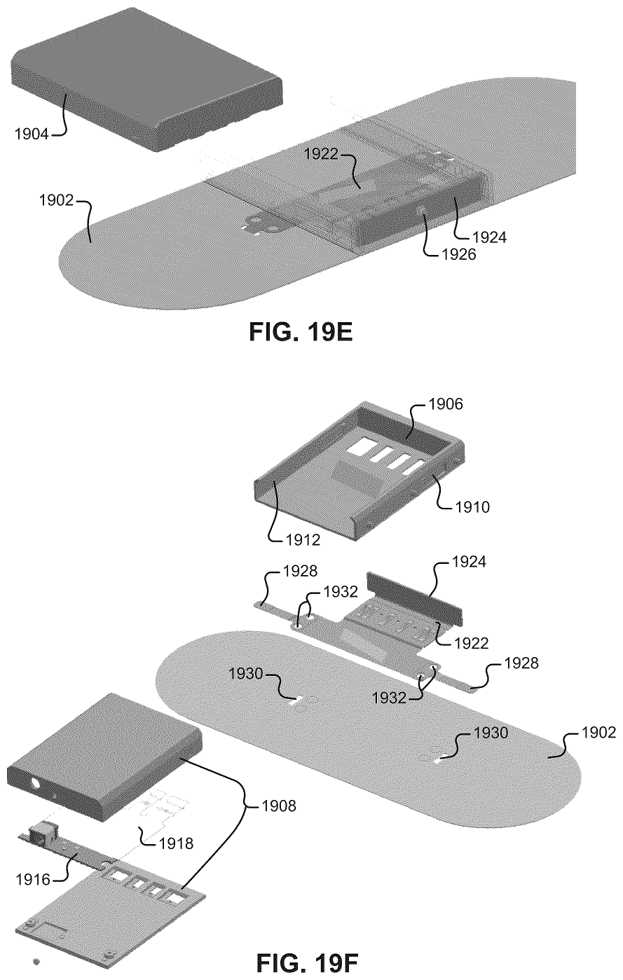

FIGS. 19A-19F illustrate a third example thermal device 100-3. The thermal device 100-3 includes thermal units 1900 attached to a package substrate 1902. The thermal units 1900 are illustrated in FIGS. 19C-19D. The thermal units are omitted from FIGS. 19E-19F in order to highlight other components of the thermal device 100-3.

The thermal device 100-3 includes a removable battery housing 1904. The battery housing 1904 includes a battery (not shown). In some implementations, the battery housing 1904 may also include device electronics. Accordingly, the battery housing 1904 may also be referred to as a “battery and electronics housing 1904.” The user may remove/replace the battery housing 1904. For example, the user may replace the battery housing 1904 with other battery housings including fully charged batteries and/or batteries with different capacities. In some implementations, the battery housing 1904 may have a different geometry than that illustrated in FIGS. 19A-19F. For example, a battery housing including a battery with a larger capacity may have a larger volume and/or different shape than that illustrated in FIGS. 19A-19F.

The battery housing 1904 mates with a receptacle 1906. In the example of FIG. 19C, the battery housing 1904 defines indentations 1908 that mate with retention clips 1910 included on the receptacle 1906. The user can slide the battery housing 1904 into the receptacle 1906 along rails 1912 defined by the receptacle 1906. The battery housing 1904 is seated and retained in position by the mating between the retention clips 1910 and indentations 1908. When the battery housing 1904 is seated in the receptacle 1906, the user can apply a force to the battery housing 1904 to unseat the battery housing 1904 from the receptacle 1906. For example, the user can apply a force to the battery housing 1904 that causes the indentations 1908 to spread the retention clips 1910 and then causes the battery housing 1904 to slide out of the receptacle 1906 along the rails 1912. The illustrated battery housing 1904 and receptacle 1906 are only one example retention mechanism for a removable battery housing. The battery housing may be attached and retained by other retention mechanisms, such as an electrical connector (e.g., friction between electrical contacts), a magnetic latch, a push/push mechanism (e.g., such as on a ballpoint pen), and/or a mechanical hook/latch (e.g., a user actuated connector).

The thermal device 100-3 includes thermal bridge/reservoir material 1914 that is attached to the package substrate 1902 and the thermal units 1900 on the side of the thermal device 100-3 facing away from the user's body during use. The thermal bridge/reservoir material 1914 may also provide comfort to the user during use. For example, the thermal bridge/reservoir material 1914 may even out the pressure against the user if the thermal device 100-3 is sandwiched between the user and an object (e.g., a chair back). Specifically, in FIG. 19A, the thermal bridge/reservoir material 1914 can help distribute pressure along the entire thermal device 100-3, which may otherwise be focused under the battery housing 1904 and receptacle 1906.

The thermal device 100-3 includes multiple flexible and rigid PCBs. With respect to FIG. 19D and FIG. 19F, the battery housing 1904 includes a first rigid PCB 1916 and a first flexible PCB 1918 that are connected to one another. The first rigid PCB 1916 includes a power input port 108 and a battery indicator 1920. The battery indicator 1920 may indicate a variety of statuses associated with the battery, such as the charge level of the battery and whether the battery is being charged. The first flexible PCB 1918 includes electrical traces that connect the battery to the electronics included on the first rigid PCB 1916. The first flexible PCB 1918 also includes electrical traces that connect to the electrical contacts on the second flexible PCB 1922 (e.g., FIG. 19D). The first rigid PCB 1916, the first flexible PCB 1918, and/or the battery may also include circuits similar to those included in the power module 1008 of FIG. 10.

The thermal device 100-3 includes a second rigid PCB 1924 and a second flexible PCB 1922 that are connected to one another. The second rigid PCB 1924 includes device electronics described herein, such as electronics included in the communication module 1004, processing module 1002, memory 1020, temperature sensing module 1012, thermal control module 1010, and interface module 1006. The LED 1926 on the thermal device 100-3 may indicate if the thermal device 100-3 is turned on, if it is connected to a user device 104 (e.g., via Bluetooth), if it is heating/cooling, and/or the state of the battery.

The second flexible PCB 1922 can be attached to the package substrate 1902 in a variety of ways. For example, the second flexible PCB 1922 can be bonded to the package substrate 1902 using adhesive bonding, heat welding, ultrasonic welding, mechanical attachments, or other technique. The second flexible PCB 1922 includes temperature sensors 1928 that extend through openings 1930 defined in the package substrate 1902. The temperature sensors 1928 are positioned between the package substrate 1902 and the user during use. The second flexible PCB 1922 also includes electrical contacts 1932 that solder to electrical contacts of thermal elements 1900.

The second flexible PCB 1922 includes electrical contacts 1934 (e.g., 6 illustrated contacts) that electrically couple the battery and electronics included in the battery housing 1904 to the device electronics included on the second flexible PCB 1922 and the second rigid PCB 1924. For example, the contacts 1934 may deliver power from the battery to the second flexible PCB 1922 and the second rigid PCB 1924. The electrical contacts 1934 may also provide for communication between components included in the battery housing 1904 and components on the receptacle side of the thermal device 100-3. For example, the contacts 1934 may allow electronics on the second rigid PCB 1924 to determine the battery serial number/ID, the battery size, the state of charge, the battery temperature, the battery usage time, and other data.

The arrangement of PCBs and device electronics described with respect to FIGS. 19A-19F is only one example arrangement of PCBs and device electronics. In other examples, the thermal device 100-3 may include other arrangements of PCBs and device electronics. For example, the thermal device 100-3 may include other arrangements of flexible and/or rigid PCBs. As another example, the battery housing 1904 may include additional device electronics, such as device electronics included in the communication module 1004, processing module 1002, memory 1020, temperature sensing module 1012, thermal control module 1010, and interface module 1006.

Note that the thermal device 100-3 does not include a manual user input button. For example, the thermal device 100-3 does not include an on/off button for turning the thermal device 100-3 on/off. Instead of controlling the thermal device 100-3 using manual buttons included on the thermal device 100-3, the user may control the thermal device 100-3 via the user device 104. For example, the user may interact with a GUI on the user device 104 to turn the thermal device 100-3 on/off or place the thermal device 100-3 in a standby/sleep mode.

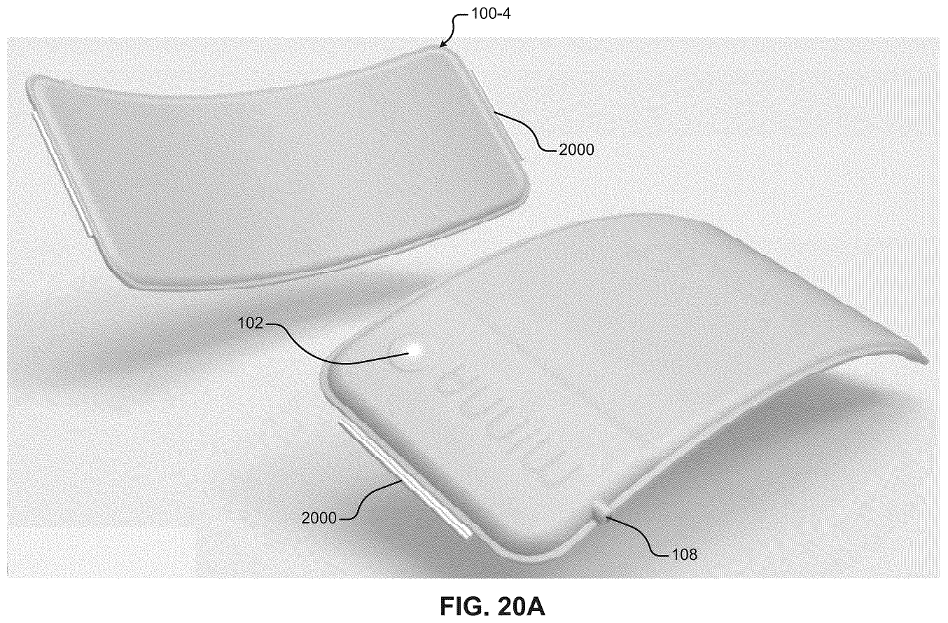

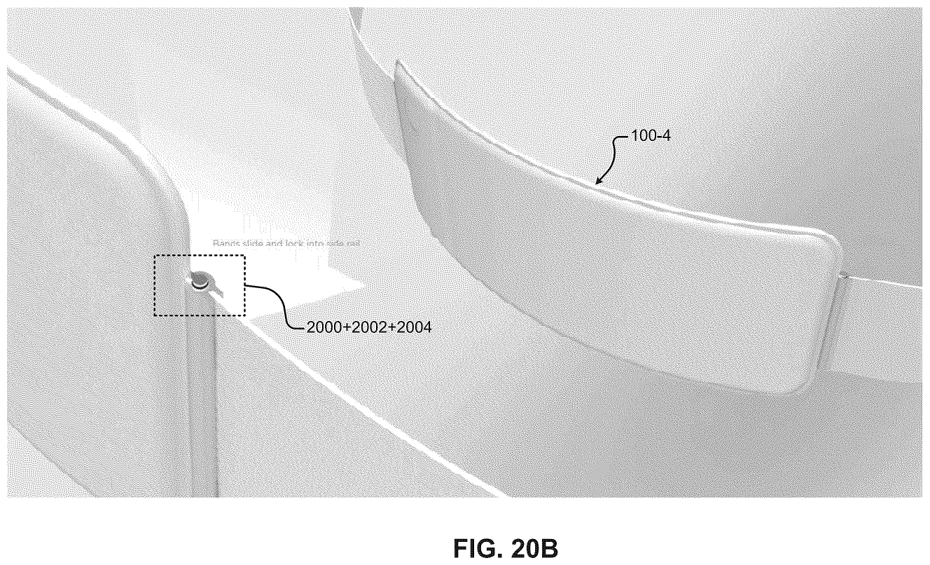

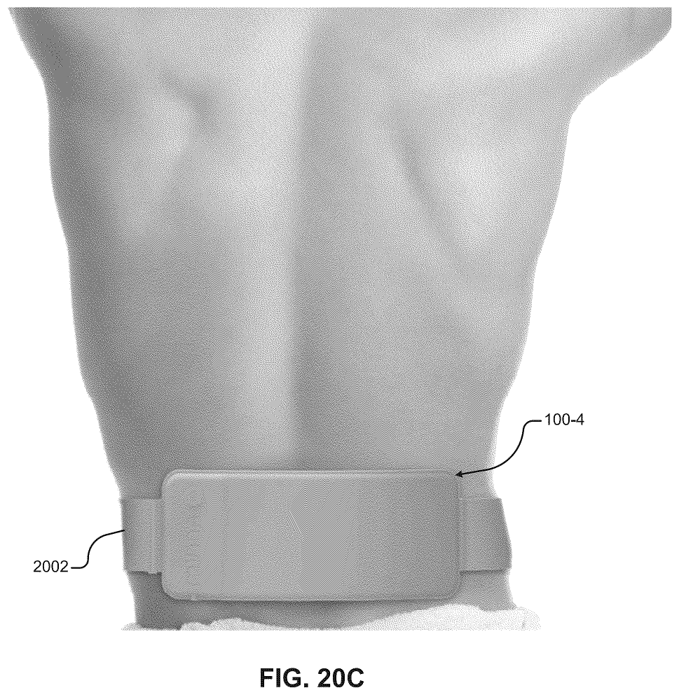

FIGS. 20A-20C illustrate a fourth thermal device 100-4. The fourth thermal device package can include one or more thermal/heating units arranged in any manner throughout the package. The fourth thermal device 100-4 can be applied to different parts of the user's body (e.g., the back or stomach). The fourth device package can include one or more connectors 2000 (device connectors) that are configured to connect to a belt 2002 having connectors 2004 (belt connectors) that mate with the device connectors 2000 of the fourth device package (see FIG. 20B). With respect to FIG. 20A, the fourth thermal device can include a user input button 102 (e.g., an on/off button) and a power input port 108.

The fourth thermal device 100-4 of FIGS. 20A-20C may include similar layers as the second thermal device 100-2, such as the encapsulation layers, package substrate, thermal/heating units, and a thermal reservoir/bridge layer. The arrangement of the components within the fourth thermal device 100-4 may be different than the arrangement of components within the second thermal device 100-2. For example, the battery, user input button, and power input port of the fourth thermal device 100-4 may be offset to one side, whereas these components are centrally located in the second thermal device 100-2. In some implementations, the fourth thermal device 100-4 may also include an adhesive layer that may be attached to the encapsulation bottom cover.

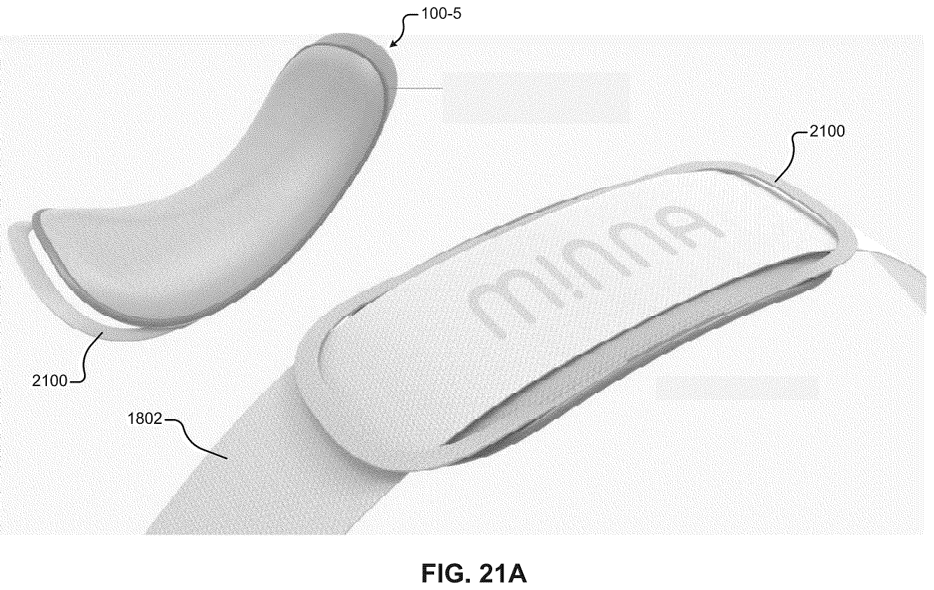

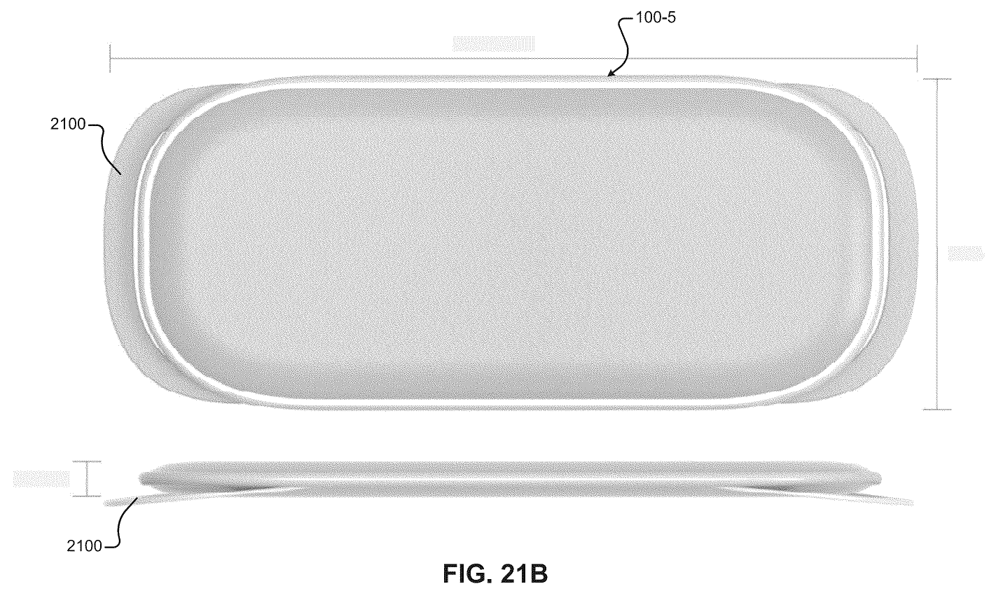

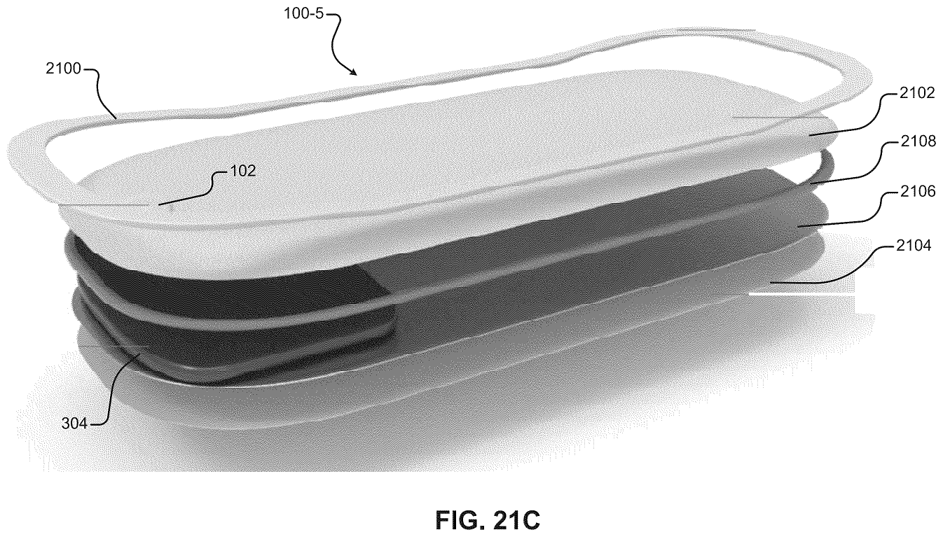

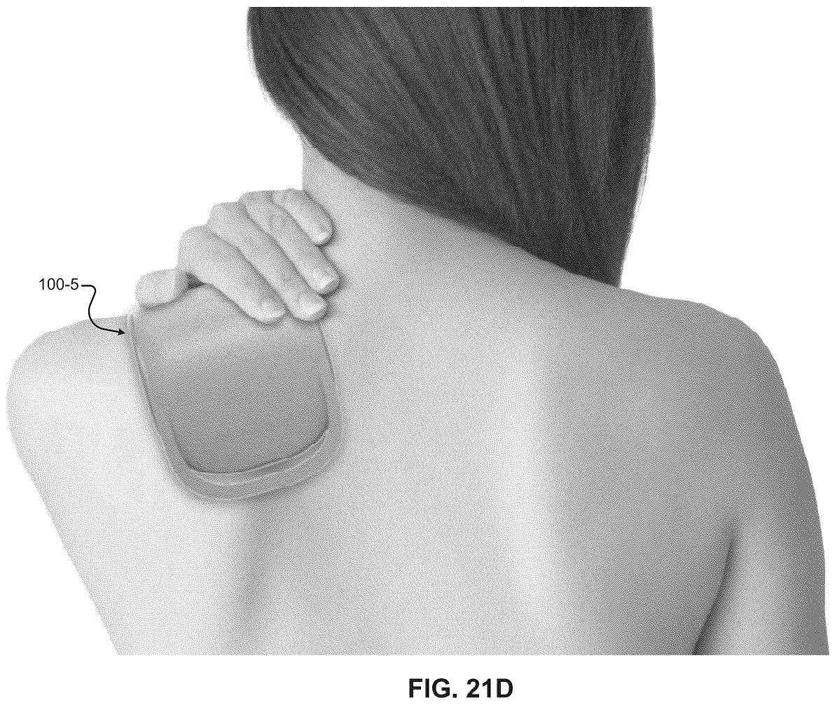

FIGS. 21A-21D illustrate a fifth thermal device 100-5. The fifth thermal device package can include one or more thermal/heating units arranged in any manner throughout the package. The fifth thermal device 100-5 can be applied to different parts of the user's body (e.g., see FIG. 21D). The fifth device package can include one or more belt loops 2100. The belt loops 2100 of the fifth device package, which are located at the edges of the fifth device package, may be integrated with the encapsulation top cover. The fifth thermal device 100-5 can include a user input button 102 (e.g., an on/off button) and a power input port (not illustrated).

Referring to FIG. 21C, the fifth thermal device 100-5 may include similar layers as the second thermal device 100-2, such as the encapsulation layers 2102, 2104, package substrate 2106, and thermal/heating units (not illustrated). The fifth thermal device also includes a shape retention element 2108 (e.g., a moldable wire or plastically deformable material) that the user can use to form the fifth thermal device 100-5 into a shape that is maintained by the shape retention element 2108. The shape retention element 2108 may be used to shape and fix the fifth thermal device 100-5 to the user's body (e.g., around the shoulder in FIG. 21D, waist, arm, hand, leg, foot, neck, or head). For example, the shape retention element 2108 (e.g., the wire) may be pressed to conform to the user's body and maintain its shape so that the thermal device 100-5 conforms to the user's body when the user removes their hand from the thermal device 100-5. Since the belt loops 2100 are integrated into the perimeter of the fifth thermal device 100-5, the belt loops 2100 may also conform to whatever shape the fifth thermal device 100-5 takes. Although the shape retention element 2108 is included around the perimeter of the fifth thermal device 100-5, a thermal device may include shape retention elements along one or more axes of the thermal device.

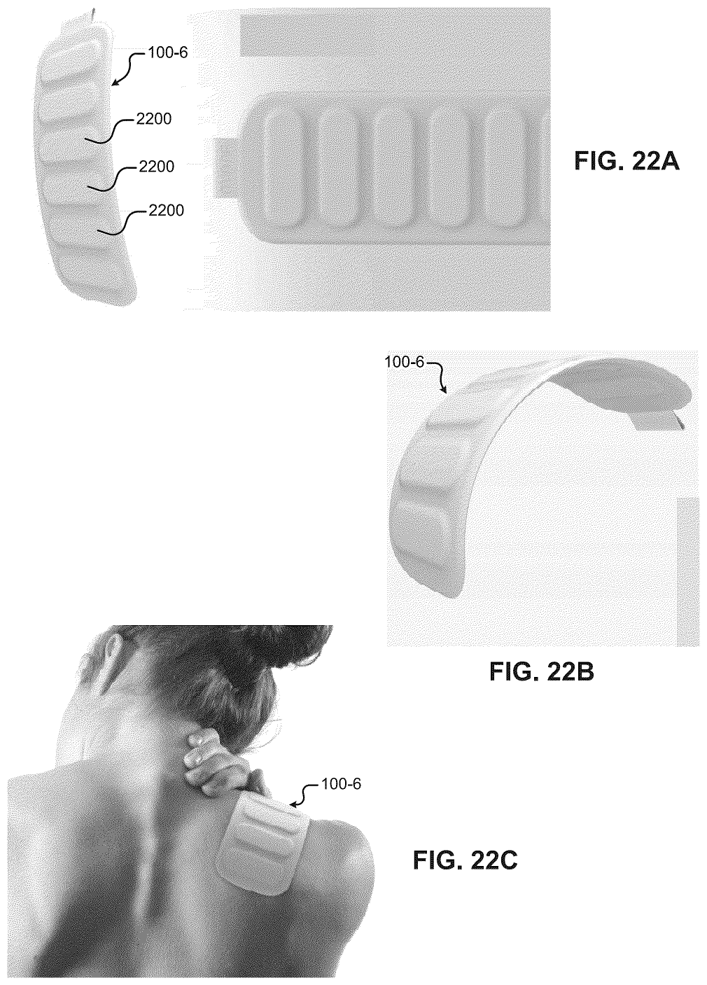

FIGS. 22A-22C illustrate a sixth thermal device 100-6 having a sixth device package. The sixth device package separates different components into different pods 2200. The pods 2200 may include different components. In some examples, one or more pods 2200 may include the battery and device electronics. In these examples, the remaining pods 2200 may include thermal/heating units. In some implementations, the thermal/heating units may be distributed throughout the full surface of the thermal device 100-6 or beneath some or all of the pods 2200. The sixth thermal device 100-6 may include similar layers as the other thermal devices, such as encapsulation layers, thermal/heating units, and an adhesive layer. Separation of the components into different pods may allow the thermal device 100-6 to easily fold/roll in one direction. The flexibility of the sixth thermal device 100-6 may help it conform to the user's body (e.g., a user's shoulder) as illustrated in FIG. 22C.

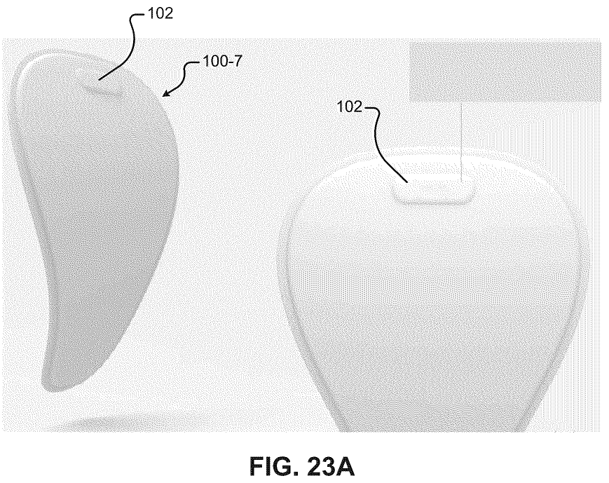

FIG. 23A illustrates a seventh thermal device 100-7 having a seventh device package. The seventh thermal device 100-7 is shaped to conform to a female's pelvic region. The seventh thermal device 100-7 may include similar layers and components as the other thermal devices, such as user input buttons, device electronics, a battery, encapsulation layers, a package substrate, thermal/heating units, and an adhesive layer. The seventh thermal device 100-7 may be flexible so that it conforms to the user's body. In some implementations, the seventh thermal device 100-7 (or any other thermal device) may be made from water repellant materials.

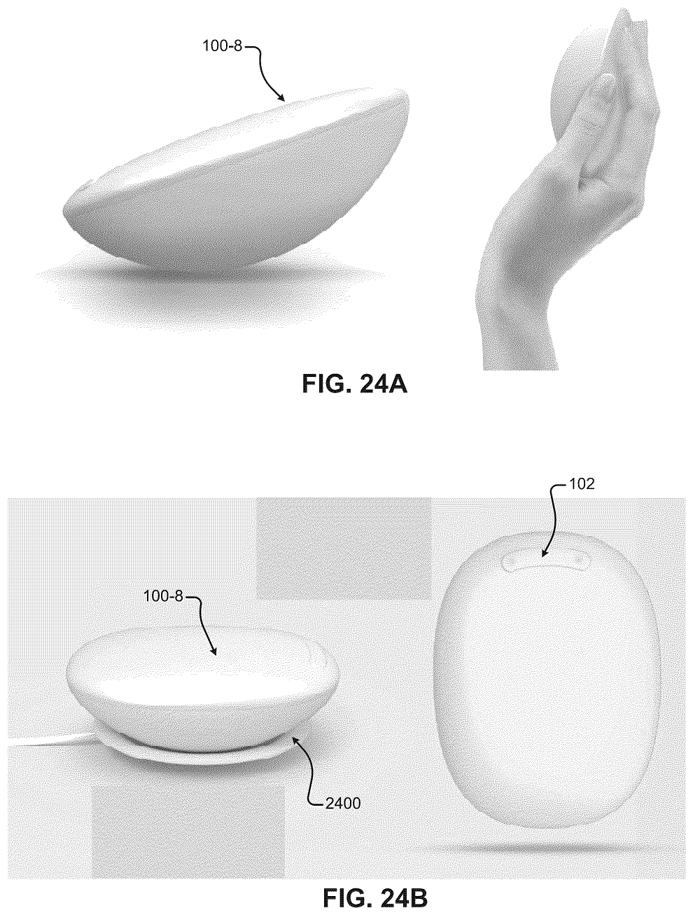

FIGS. 24A-24B illustrate an eighth thermal device 100-8. The eighth thermal device 100-8 is configured to fit into a user's hand. The flattened portion of the thermal device 100-8 includes user input buttons 102 that the user may actuate in order to increase/decrease the amount of heating/cooling generated by the eighth thermal device 100-8. The convex portion of the eighth thermal device 100-8 may act as the hot/cold side and may be soft and compliant to promote comfortable contact with the user's body. The convex portion of the eighth thermal device 100-8 may also include a thermal reservoir material in order to extend the operating time of the eighth thermal device 100-8. The eighth thermal device 100-8 may also include circuits for wireless charging. For example, the eighth thermal device 100-8 may be wirelessly charged from the wireless charging station 2400 illustrated in FIG. 24B.

FIGS. 25A-25E illustrate various sleeves and garments that may be configured to hold the thermal devices 100 described herein. FIG. 25A illustrates an example sleeve 2500 that holds the seventh thermal device 100-7. The sleeve 2500 of FIG. 25A may be fabricated from a cloth material (e.g., cotton or other fabric). In some implementations, the sleeve 2500 may be fabricated from a material that is thermally conductive. In some implementations, the sleeve 2500 may be fabricated from a breathable material. FIG. 25B illustrates another sleeve 2502. The sleeve 2502 of FIG. 25B is a weighted sleeve configured to hold the second thermal device 100-2. The weighted sleeve 2502 may apply pressure to the thermal device during use (e.g., while resting on the user).

FIGS. 25C-25E illustrate garments that are configured to hold the thermal devices. FIG. 25C is a female underwear garment 2504 including a device pouch 2506 that is shaped to hold the seventh thermal device 100-7 in the pelvic region. FIG. 25D is another underwear garment 2508 including a device pouch 2510 for holding a thermal device. Specifically, the garment 2508 of FIG. 25D includes a device pouch 2510 that holds the second thermal device 100-2 above the pubic region. FIG. 25E illustrates an additional example underwear garment 2512 that includes a device pouch 2514 for holding the second thermal device 100-2 in the user's lower back.

Although a single thermal device in communication with a user device is illustrated and described herein, in some implementations, a single user device may communicate with multiple thermal devices. For example, a user may place two thermal devices on their body and control/monitor the two thermal devices using a single user device.

Although the package substrate is illustrated herein as holding the thermal units, in some implementations, the thermal units may be held within the device package in other manners. For example, the top and bottom of the thermal units may be sandwiched between two different substrates (e.g., each similar to the package substrate). As another example, the thermal units may be attached to the top and/or bottom encapsulation layers which may serve to hold the thermal units in place.

Various examples have been described. These and other examples are within the scope of the following claims.

What is claimed is:

1. A thermal device comprising:
a thermal unit comprising a plurality of semiconductor elements sandwiched between a first thermal unit substrate and a second thermal unit substrate;
device electronics coupled to the thermal unit, wherein the device electronics are configured to operate the thermal unit in a cooling state in which the thermal unit removes heat from a first portion of a user's body via the first thermal unit substrate; and
a thermally conductive thermal bridge that is thermally coupled to the second thermal unit substrate such that the thermal bridge removes heat from the second thermal unit substrate while the thermal unit is operating in the cooling state, wherein the thermal bridge is configured to extend from the second thermal unit substrate to a second portion of the user's body that is different than the first portion of the user's body, and wherein the thermal bridge is configured to transfer heat to the second portion of the user's body while the thermal unit is operating in the cooling state.

2. The thermal device of claim 1, wherein the thermal bridge only partially surrounds the thermal unit.

3. The thermal device of claim 1, wherein the thermal bridge fully surrounds the thermal unit such that the first portion of the user's body is fully surrounded by the second portion of the user's body receiving heat from the thermal bridge.

4. The thermal device of claim 1, further comprising:
an encapsulation that includes the thermal unit, the device electronics, and the thermal bridge; and
a belt configured to hold the thermal device to the user's body.

5. The thermal device of claim 1, wherein the device electronics are configured to control power delivery to the thermal unit to control the removal of heat from the first portion of the user's body.

6. The thermal device of claim 5, further comprising a user input device configured to receive user input, wherein the user input device communicates with the device electronics, and wherein the device electronics are configured to control power delivery to the thermal unit in response to the user input received by the user input device.

7. The thermal device of claim 5, wherein the device electronics are configured to:
wirelessly receive user-input instructions from an external computing device indicating how to modify power delivery to the thermal unit; and
modify power delivery to the thermal unit in response to the received user-input instructions.

8. The thermal device of claim 5, further comprising a temperature sensor that indicates a temperature associated with the first portion of the user's body, wherein the device electronics are configured to control power delivery to the thermal unit based on the temperature indicated by the temperature sensor.

9. The thermal device of claim 1, wherein the thermal bridge includes metal.

10. The thermal device of claim 9, wherein the thermal bridge includes a metal mesh.

11. The thermal device of claim 1, wherein the thermal bridge is flexible.

12. The thermal device of claim 1, further comprising a battery that delivers power to the device electronics and the thermal unit.

13. The thermal device of claim 1, further comprising a thermal reservoir material in contact with the thermal bridge, wherein the thermal reservoir material acts as a heat sink that receives thermal energy from the thermal bridge.

14. The thermal device of claim 13, wherein the thermal reservoir material is included on the thermal bridge such that the thermal bridge separates the thermal reservoir material from the second thermal unit substrate.

15. The thermal device of claim 13, wherein the thermal bridge defines one or more cavities that hold the thermal reservoir material.

16. The thermal device of claim 13, wherein the thermal reservoir material includes a phase-change material.

17. The thermal device of claim 1, further comprising a thermally conductive body contact layer attached to the first thermal unit substrate such that the first thermal unit substrate removes heat from the first portion of the user's body through the body contact layer.

18. The thermal device of claim 1, further comprising one or more additional thermal units configured to remove heat from the user's body, wherein the one or more additional thermal units are coupled to the thermal bridge such that the thermal bridge removes heat from the one or more additional thermal units and transfers heat from the one or more additional thermal units to the second portion of the user's body.

19. The thermal device of claim 1, further comprising an additional thermal unit that is thermally coupled to the thermal bridge, wherein the additional thermal unit removes heat from a third portion of the user's body that is separate from the first portion and the second portion, and wherein the thermal bridge is configured to transfer heat to a fourth portion of the user's body that is between the third portion of the user's body and the first portion of the user's body.

20. The thermal device of claim 1, wherein the device electronics are configured to transition the first thermal unit from operating in the cooling state to operating in a heating state, wherein the first thermal unit transfers heat to the first portion of the user's body via the first thermal unit substrate while operating in the heating state.
